SEQUENCE LISTING (22 sequences)

SEQ ID NO: 1 - protein, 1300 aa, Artificial Sequence
  Modified SARS CoV-2 eS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTGYIPEAPRDGQAYVRKDGEWVLLSTFL

SEQ ID NO: 2 - protein, 222 aa, Artificial Sequence
  SARS-CoV2 M Protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 3 - protein, 400 aa, Artificial Sequence
  SARS-CoV2 N Protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADL

SEQ ID NO: 4 - protein, 720 aa, Artificial Sequence
  Positions 1-1296 of SARS CoV-2 S Protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTI

SEQ ID NO: 5 - protein, 1296 aa, Artificial Sequence
  Modified SARS CoV-2 eS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLALAYGSQGNLNPLINEISKIISAAGNFDVKEERAAASLLQLSGNASDFSYGRNSITLTASA

SEQ ID NO: 6 - DNA, 11122 bp, Artificial Sequence
  Ty21a-eS Construct
cccgttcctcattgatttgattgctaacgtcatgagcattactctatcattccagaatgcctcaatgaacaagttgtttgagaaagagtgtcgcaatgttgcaaccagagcccttaaatatgtacgccagaagaaaactgaagggcgtctggatgaagcattgtctgtattgattagcctgaaacgaattgagcctgatgtttctcgtctgatgcgtgaatataagcaaattatcagattatttaatgagtcacggaaggatggcggtagcactatcacgtcttatgaacatctagactatgcgaaaaaattactcgtttttgatagcgaaaatgcctatgccttgaaatatgccgcattaaatgcaatgcatttacgcgactacacgcaggctttgcagtattggcagcgactggagaaagtgaatggaccaacggagccggtgacaaggcagatctcgacctgcataaccgcattacaaaaaaatacatcagggaagtcgtaacccggggtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatatgcattaatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactgtcgaccaaaaaaatattgacaacataaaaaactttgtgttatacttgtaacggactctagagggtattaataatgaaaggagaggacatgagctcatgtttgtgtttctggtgctgctgccgctggtgagcagccagtgcgtgaacctgaccacccgcacccagctgccgccggcgtataccaacagctttacccgcggcgtgtattatccggataaagtgtttcgcagcagcgtgctgcatagcacccaggatctgtttctgccgttttttagcaacgtgacctggtttcatgcgattcatgtgagcggcaccaacggcaccaaacgctttgataacccggtgctgccgtttaacgatggcgtgtattttgcgagcaccgaaaaaagcaacattattcgcggctggatttttggcaccaccctggatagcaaaacccagagcctgctgattgtgaacaacgcgaccaacgtggtgattaaagtgtgcgaatttcagttttgcaacgatccgtttctgggcgtgtattatcataaaaacaacaaaagctggatggaaagcgaatttcgcgtgtatagcagcgcgaacaactgcacctttgaatatgtgagccagccgtttctgatggatctggaaggcaaacagggcaactttaaaaacctgcgcgaatttgtgtttaaaaacattgatggctattttaaaatttatagcaaacataccccgattaacctggtgcgcgatctgccgcagggctttagcgcgctggaaccgctggtggatctgccgattggcattaacattacccgctttcagaccctgctggcgctgcatcgcagctatctgaccccgggcgatagcagcagcggctggaccgcgggcgcggcggcgtattatgtgggctatctgcagccgcgcacctttctgctgaaatataacgaaaacggcaccattaccgatgcggtggattgcgcgctggatccgctgagcgaaaccaaatgcaccctgaaaagctttaccgtggaaaaaggcatttatcagaccagcaactttcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaactttaactttaacggcctgaccggcaccggcgtgctgaccgaaagcaacaaaaaatttctgccgtttcagcagtttggccgcgatattgcggataccaccgatgcggtgcgcgatccgcagaccctggaaattctggatattaccccgtgcagctttggcggcgtgagcgtgattaccccgggcaccaacaccagcaaccaggtggcggtgctgtatcagggcgtgaactgcaccgaagtgccggtggcgattcatgcggatcagctgaccccgacctggcgcgtgtatagcaccggcagcaacgtgtttcagacccgcgcgggctgcctgattggcgcggaacatgtgaacaacagctatgaatgcgatattccgattggcgcgggcatttgcgcgagctatcagacccagaccaacagcccgggcagcgcgagcagcgtggcgagccagagcattattgcgtataccatgagcctgggcgcggaaaacagcgtggcgtatagcaacaacagcattgcgattccgaccaactttaccattagcgtgaccaccgaaattctgccggtgagcatgaccaaaaccagcgtggattgcaccatgtatatttgcggcgatagcaccgaatgcagcaacctgctgctgcagtatggcagcttttgcacccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcgggcgcggcgctgcagattccgtttgcgatgcagatggcgtatcgctttaacggcattggcgtgacccagaacgtgctgtatgaaaaccagaaactgattgcgaaccagtttaacagcgcgattggcaaaattcaggatagcctgagcagcaccgcgagcgcgctgggcaaactgcaggatgtggtgaaccagaacgcgcaggcgctgaacaccctggtgaaacagctgagcagcaactttggcgcgattagcagcgtgctgaacgatattctgagccgcctggatccgccggaagcggaagtgcagattgatcgcctgattaccggccgcctgcagagcctgcagacctatgtgacccagcagctgattcgcgcggcggaaattcgcgcgagcgcgaacctggcggcgaccaaaatgagcgaatgcgtgctgggccagagcaaacgcgtggatttttgcggcaaaggctatcatctgatgagctttccgcagagcgcgccgcatggcgtggtgtttctgcatgtgacctatgtgccggcgcaggaaaaaaactttaccaccgcgccggcgatttgccatgatggcaaagcgcattttccgcgcgaaggcgtgtttgtgagcaacggcacccattggtttgtgacccagcgcaacttttatgaaccgcagattattaccaccgataacacctttgtgagcggcaactgcgatgtggtgattggcattgtgaacaacaccgtgtatgatccgctgcagccggaactggatagctttaaagaagaactggataaatattttaaaaaccataccagcccggatgtggatctgggcgatattagcggcattaacgcgagcgtggtgaacattcagaaagaaattgatcgcctgaacgaagtggcgaaaaacctgaacgaaagcctgattgatctgcaggaactgggcaaatatgaacagggctatattccggaagcgccgcgcgatggccaggcgtatgtgcgcaaagatggcgaatgggtgctgctgagcacctttctggcattagcctatggaagtcagggtaatcttaatccattaattaatgaaatcagcaaaatcatttcagctgcaggtaattttgatgttaaagaggaaagagcggccgcgtctttattgcagttgtccggtaatgccagtgatttttcatatggacggaactcaataactttgacagcatcagcataagagctctttattaatttaaataatagcaatcttactgggctgtgccacataagattgctatttttttggagtcataatggattcttgtcataaaattgattatgggttatacgccctggagattttagcccaataccataacgtctctgttaacccggaagaaattaaacatagatttgacacagacgggactggtctgggattaacgtcatggttgcttgctgcgaaatctttagaactaaaggtaaaacaggtaaaaaaaacaattgaccgattaaactttatttctctgcccgcattagtctggagagaggatggatgtcattttattctgactaaagtcagtaaagaagcaaacagatatcttatttttgatctggagcagcgaaatccccgtgttctcgaacagtctgagtttgaggcgttatatcaggggcatattattcttattgcttcccgttcttctgttaccgggaaactggcaaaatttgactttacctggtttattcctgccattataaaatacaggaaaatatttattgaaacccttgttgtatctgtttttttacaattatttgcattaataaccccccttttttttcaggtggttatggacaaagtattagtacacagggggttttcaacccttaatgttattactgtcgcattatctgttgtggtggtgtttgagattatactcagcggtttaagaacttacatttttgcacatagtacaagtcggattgatgttgagttgggtgccaaactcttccggcatttactggcgctaccgatctcttattttgagagtcgtcgtgttggtgatactgttgccagggtaagagaattagaccagatccgtaatttcctgacaggacaggcattaacatctgttctggacttattattttcattcatattttttgcggtaatgtggtattacagcccaaagcttactctggtgatcttattttcgctgccctgttatgctgcatggtctgtttttattagccccattttgcgacgtcgccttgatgataagttttcacggaatgcggataatcaatctttcctggtggaatcagtcacggcgattaacactataaaagctatggcagtctcacctcagatgacgaacatatgggacaaacaattggcaggatatgttgctgcaggctttaaagtgacagtattagccaccattggtcaacaaggaatacagttaatacaaaagactgttatgatcatcaacctgtggttgggtgcacacctggttatttccggggatttaagtattggtcagttaattgcttttaatatgcttgcaggtcagattgttgcaccggttattcgccttgcacaaatctggcaggatttccagcaggttggtatatcagttacccgccttggtgatgtgcttaactctccaactgaaagttatcatgggaaactggcattaccggaaattaatggtgatatcacttttcgtaatatccggtttcgctataagcctgactctccggttattttagataatatcaatctcagtattaagcagggggaggttattggtattgtcggacgttctggttcaggaaaaagcacattaactaaattaattcaacgtttttatattcctgaaaatggccaggtcttaattgatggacatgatcttgcgttggccgatcctaactggttacgtcgtcaggtgggggttgtgttgcaggacaatgtgctgcttaatcgcagtattattgataatatttcactggctaatcctggcatgtccgtcgaaaaagttatttatgcagcgaaattagcaggtgctcatgattttatttctgaattgcgtgaggggtataacaccattgtcggggaacagggggcaggattatccggaggtcaacgtcaacgcatcgcaattgcaagggcgctggtgaacaaccctaaaatactcatctttgatgaagcaaccagtgctctggattatgagtcggagcatgtcatcatgcgcaatatgcacaaaatatgtaagggcagaacggttataatcattgctcatcgtctgtctacagtaaaaaatgcagaccgcattattgtcatggaaaaagggaaaattgttgaacagggtaaacataaggagctgctttctgaaccggaaagtttatacagttacttatatcagttacagtcagactaacagaaagaacagaagaatatgaaaacatggttaatggggttcagcgagttcctgttgcgctataaacttgtctggagtgaaacatggaaaatccggaagcagttagatactccggtacgtgaaaaggacgaaaatgaattcttacccgctcatctggaattaattgaaacgccggtatccagacggccgcgtctggttgcttattttattatggggtttctggttattgctttcattttatctgttttaggccaggtggaaattgttgccactgcaaatgggaaattaacactcagtgggcgtagcaaagaaattaaacctattgaaaactcgatagttaaagaaattatcgtaaaagaaggagagtcagtccggaaaggggatgtgttattaaagcttacagcgctgggagctgaagctgatacgttaaaaacgcagtcatcactgttacaggccaggctggaacaaattcggtatcaaattctgagccggtcaattgaattaaataaacttcctgaactgaaacttcctgatgagccttattttcagaatgtatctgaagaggaagtactgcgtttaacttctttgataaaagaacagttttccacatggcaaaatcagaagtatcaaaaagaactgaatctggataagaaaagagcagagcgattaacaatacttgcccgtataaaccgttatgaaaatgtatcgagggttgaaaaaagccgtctggatgatttcaggagcctgttgcataaacaggcaattgcaaaacatgctgtacttgagcaggagaataaatatgttgaggcagcaaatgaattacgggtttataaatcgcaactggagcaaattgagagtgagatattgtctgcaaaagaagaatatcagcttgtcacgcagctttttaaaaatgaaattttagacaagctaagacaaacaacagacagcattgagttattaactctggagttagagaaaaatgaagagcgtcaacaggcttcagtaatcagggcccctgtttcgggaaaagttcagcaactgaaggttcatactgaaggtggggttgttacaacagcggaaacactgatggtcatcgttccggaagatgacacgctggaggttactgctctggtacaaaataaagatattggttttattaacgtcgggcagaatgccatcattaaagtggaggcttttccttacacccgatatggttatctggtgggtaaggtaaaaaatataaatttagatgcaatagaagaccagaaactgggactcgtttttaatgtcattgtttctgttgaagagaatgatttgtcaaccgggaataagcacattccattaagctcgggtatggctgtcactgcagaaataaagactggaatgcgaagcgtaatcagttatcttcttagtcccctggaagaatctgtaacagaaagtttacatgagcgttaagtctcagagcagcggtatccggctcatatcttctcctgtcagatccaagggcgaattccagcacactggcggccgttactagtggatccaccggtctcgaggggcgcgccggtaccgaattctaattaatgggcatcatttttcagctatttcatttataaaataagttatgaaaaaaatcatcatattactaacgacatttttcctgctttcgggatgcactattcccagggcggtatttaaatccagccttattaatcaggacgatcctcgttataatctggtcgaagtcacgccgacattaaaactaagcgctcccgatactgtgccgaaaactattgtcgatccggtttttgccgcaaataactggcactggacatctttggctaaaggcgatgtgctgcatatcactattttatcctcgggcggggctggatatttatccaataacgcgagcggcgaccgtgcggattttgaaaatattcttgtgactgacagtaataccgttcaggtgccttatgccgggacaatcccggtttctggattggatgtgacgcaactggctgatgagatcaaaaagcgactttcgcgcgttgtcctgaatcctcaggtgattgtgacacttaccgcccgcaccggagccatggtgacggtcgagggcagcgggaaaacgggtcgataccctctcgaacagagtatgaatcgtctgagtcatcttttggcaacggcagtggcagtagaaaataccagcacagatatgatggaagttcacgtgacgcgccagcagcattatttcactgcgcgactgtcagatatttatcagtatcccggattggatattgccttacagccggatgaccgtatcactctgcgtcaagtgaccgagtatgttaatgtgctcggcgcagcaggtgttcaggggaaacatgctctggttcagcgtcattccagcgtcgtggatgctttggcgctcgcgaaaggattaaatgacaatcttgccgatccgcaagcgatttttctgtacaagcataacgaagcggaacaggcgaaacagcagatgcgtaagctgaatatctatcatgtcgatatgagccaacctaactcggtgtttttagcgcaggccatacgagtggacaacggcgatgttatctatatctcgaacgcctctttgactgatttcgctaaggtgaaagccgcattcgatagctttttgacccgcggcactaattctttctaa

SEQ ID NO: 7 - DNA, 9342 bp, Artificial Sequence
  Ty21a-M-N Construct
cccgttcctcattgatttgattgctaacgtcatgagcattactctatcattccagaatgcctcaatgaacaagttgtttgagaaagagtgtcgcaatgttgcaaccagagcccttaaatatgtacgccagaagaaaactgaagggcgtctggatgaagcattgtctgtattgattagcctgaaacgaattgagcctgatgtttctcgtctgatgcgtgaatataagcaaattatcagattatttaatgagtcacggaaggatggcggtagcactatcacgtcttatgaacatctagactatgcgaaaaaattactcgtttttgatagcgaaaatgcctatgccttgaaatatgccgcattaaatgcaatgcatttacgcgactacacgcaggctttgcagtattggcagcgactggagaaagtgaatggaccaacggagccggtgacaaggcagatctcgacctgcataaccgcattacaaaaaaatacatcagggaagtcgtaacccggggtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatatgcattaatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactgtcgaccaaaaaaatattgacaacataaaaaactttgtgttatacttgtaacggactctagagggtattaataatgaaaggagaggacatgagctccctaggatgagcgataacggcccgcagaaccagcgcaacgcgccgcgcattacctttggcggcccgagcgatagcaccggcagcaaccagaacggcgaacgcagcggcgcgcgcagcaaacagcgccgcccgcagggcctgccgaacaacaccgcgagctggtttaccgcgctgacccagcatggcaaagaagatctgaaatttccgcgcggccagggcgtgccgattaacaccaacagcagcccggatgatcagattggctattatcgccgcgcgacccgccgcattcgcggcggcgatggcaaaatgaaagatctgagcccgcgctggtatttttattatctgggcaccggcccggaagcgggcctgccgtatggcgcgaacaaagatggcattatttgggtggcgaccgaaggcgcgctgaacaccccgaaagatcatattggcacccgcaacccggcgaacaacgcggcgattgtgctgcagctgccgcagggcaccaccctgccgaaaggcttttatgcggaaggcagccgcggcggcagccaggcgagcagccgcagcagcagccgcagccgcaacagcagccgcaacagcaccccgggcagcagccgcggcaccagcccggcgcgcatggcgggcaacggcggcgatgcggcgctggcgctgctgctgctggatcgcctgaaccagctggaaagcaaaatgagcggcaaaggccagcagcagcagggccagaccgtgaccaaaaaaagcgcggcggaagcgagcaaaaaaccgcgccagaaacgcaccgcgaccaaagcgtataacgtgacccaggcgtttggccgccgcggcccggaacagacccagggcaactttggcgatcaggaactgattcgccagggcaccgattataaacattggccgcagattgcgcagtttgcgccgagcgcgagcgcgttttttggcatgagccgcattggcatggaagtgaccccgagcggcacctggctgacctataccggcgcgattaaactggatgataaagatccgaactttaaagatcaggtgattctgctgaacaaacatattgatgcgtataaaacctttccgccgaccgaaccgaaaaaagataaaaaaaaaaaagcggatgaaacccaggcgctgccgcagcgccagaaaaaacagcagaccgtgaccctgctgccggcggcggatctggatgattttagcaaacagctgcagcagagcatgagcagcgcggatagcacccaggcgcctaggcttaagatggcggatagcaacggcaccattaccgtggaagaactgaaaaaactgctggaacagtggaacctggtgattggctttctgtttctgacctggatttgcctgctgcagtttgcgtatgcgaaccgcaaccgctttctgtatattattaaactgatttttctgtggctgctgtggccggtgaccctggcgtgctttgtgctggcggcggtgtatcgcattaactggattaccggcggcattgcgattgcgatggcgtgcctggtgggcctgatgtggctgagctattttattgcgagctttcgcctgtttgcgcgcacccgcagcatgtggagctttaacccggaaaccaacattctgctgaacgtgccgctgcatggcaccattctgacccgcccgctgctggaaagcgaactggtgattggcgcggtgattctgcgcggccatctgcgcattgcgggccatcatctgggccgctgcgatattaaagatctgccgaaagaaattaccgtggcgaccagccgcaccctgagctattataaactgggcgcgagccagcgcgtggcgggcgatagcggctttgcggcgtatagccgctatcgcattggcaactataaactgaacaccgatcatagcagcagcagcgataacattgcgctgctggtgcagcttaaggcattagcctatggaagtcagggtaatcttaatccattaattaatgaaatcagcaaaatcatttcagctgcaggtaattttgatgttaaagaggaaagagcggccgcgtctttattgcagttgtccggtaatgccagtgatttttcatatggacggaactcaataactttgacagcatcagcataagagctctttattaatttaaataatagcaatcttactgggctgtgccacataagattgctatttttttggagtcataatggattcttgtcataaaattgattatgggttatacgccctggagattttagcccaataccataacgtctctgttaacccggaagaaattaaacatagatttgacacagacgggactggtctgggattaacgtcatggttgcttgctgcgaaatctttagaactaaaggtaaaacaggtaaaaaaaacaattgaccgattaaactttatttctctgcccgcattagtctggagagaggatggatgtcattttattctgactaaagtcagtaaagaagcaaacagatatcttatttttgatctggagcagcgaaatccccgtgttctcgaacagtctgagtttgaggcgttatatcaggggcatattattcttattgcttcccgttcttctgttaccgggaaactggcaaaatttgactttacctggtttattcctgccattataaaatacaggaaaatatttattgaaacccttgttgtatctgtttttttacaattatttgcattaataaccccccttttttttcaggtggttatggacaaagtattagtacacagggggttttcaacccttaatgttattactgtcgcattatctgttgtggtggtgtttgagattatactcagcggtttaagaacttacatttttgcacatagtacaagtcggattgatgttgagttgggtgccaaactcttccggcatttactggcgctaccgatctcttattttgagagtcgtcgtgttggtgatactgttgccagggtaagagaattagaccagatccgtaatttcctgacaggacaggcattaacatctgttctggacttattattttcattcatattttttgcggtaatgtggtattacagcccaaagcttactctggtgatcttattttcgctgccctgttatgctgcatggtctgtttttattagccccattttgcgacgtcgccttgatgataagttttcacggaatgcggataatcaatctttcctggtggaatcagtcacggcgattaacactataaaagctatggcagtctcacctcagatgacgaacatatgggacaaacaattggcaggatatgttgctgcaggctttaaagtgacagtattagccaccattggtcaacaaggaatacagttaatacaaaagactgttatgatcatcaacctgtggttgggtgcacacctggttatttccggggatttaagtattggtcagttaattgcttttaatatgcttgcaggtcagattgttgcaccggttattcgccttgcacaaatctggcaggatttccagcaggttggtatatcagttacccgccttggtgatgtgcttaactctccaactgaaagttatcatgggaaactggcattaccggaaattaatggtgatatcacttttcgtaatatccggtttcgctataagcctgactctccggttattttagataatatcaatctcagtattaagcagggggaggttattggtattgtcggacgttctggttcaggaaaaagcacattaactaaattaattcaacgtttttatattcctgaaaatggccaggtcttaattgatggacatgatcttgcgttggccgatcctaactggttacgtcgtcaggtgggggttgtgttgcaggacaatgtgctgcttaatcgcagtattattgataatatttcactggctaatcctggcatgtccgtcgaaaaagttatttatgcagcgaaattagcaggtgctcatgattttatttctgaattgcgtgaggggtataacaccattgtcggggaacagggggcaggattatccggaggtcaacgtcaacgcatcgcaattgcaagggcgctggtgaacaaccctaaaatactcatctttgatgaagcaaccagtgctctggattatgagtcggagcatgtcatcatgcgcaatatgcacaaaatatgtaagggcagaacggttataatcattgctcatcgtctgtctacagtaaaaaatgcagaccgcattattgtcatggaaaaagggaaaattgttgaacagggtaaacataaggagctgctttctgaaccggaaagtttatacagttacttatatcagttacagtcagactaacagaaagaacagaagaatatgaaaacatggttaatggggttcagcgagttcctgttgcgctataaacttgtctggagtgaaacatggaaaatccggaagcagttagatactccggtacgtgaaaaggacgaaaatgaattcttacccgctcatctggaattaattgaaacgccggtatccagacggccgcgtctggttgcttattttattatggggtttctggttattgctttcattttatctgttttaggccaggtggaaattgttgccactgcaaatgggaaattaacactcagtgggcgtagcaaagaaattaaacctattgaaaactcgatagttaaagaaattatcgtaaaagaaggagagtcagtccggaaaggggatgtgttattaaagcttacagcgctgggagctgaagctgatacgttaaaaacgcagtcatcactgttacaggccaggctggaacaaattcggtatcaaattctgagccggtcaattgaattaaataaacttcctgaactgaaacttcctgatgagccttattttcagaatgtatctgaagaggaagtactgcgtttaacttctttgataaaagaacagttttccacatggcaaaatcagaagtatcaaaaagaactgaatctggataagaaaagagcagagcgattaacaatacttgcccgtataaaccgttatgaaaatgtatcgagggttgaaaaaagccgtctggatgatttcaggagcctgttgcataaacaggcaattgcaaaacatgctgtacttgagcaggagaataaatatgttgaggcagcaaatgaattacgggtttataaatcgcaactggagcaaattgagagtgagatattgtctgcaaaagaagaatatcagcttgtcacgcagctttttaaaaatgaaattttagacaagctaagacaaacaacagacagcattgagttattaactctggagttagagaaaaatgaagagcgtcaacaggcttcagtaatcagggcccctgtttcgggaaaagttcagcaactgaaggttcatactgaaggtggggttgttacaacagcggaaacactgatggtcatcgttccggaagatgacacgctggaggttactgctctggtacaaaataaagatattggttttattaacgtcgggcagaatgccatcattaaagtggaggcttttccttacacccgatatggttatctggtgggtaaggtaaaaaatataaatttagatgcaatagaagaccagaaactgggactcgtttttaatgtcattgtttctgttgaagagaatgatttgtcaaccgggaataagcacattccattaagctcgggtatggctgtcactgcagaaataaagactggaatgcgaagcgtaatcagttatcttcttagtcccctggaagaatctgtaacagaaagtttacatgagcgttaagtctcagagcagcggtatccggctcatatcttctcctgtcagatccaagggcgaattccagcacactggcggccgttactagtggatccaccggtctcgaggggcgcgccggtaccgaattctaattaatgggcatcatttttcagctatttcatttataaaataagttatgaaaaaaatcatcatattactaacgacatttttcctgctttcgggatgcactattcccagggcggtatttaaatccagccttattaatcaggacgatcctcgttataatctggtcgaagtcacgccgacattaaaactaagcgctcccgatactgtgccgaaaactattgtcgatccggtttttgccgcaaataactggcactggacatctttggctaaaggcgatgtgctgcatatcactattttatcctcgggcggggctggatatttatccaataacgcgagcggcgaccgtgcggattttgaaaatattcttgtgactgacagtaataccgttcaggtgccttatgccgggacaatcccggtttctggattggatgtgacgcaactggctgatgagatcaaaaagcgactttcgcgcgttgtcctgaatcctcaggtgattgtgacacttaccgcccgcaccggagccatggtgacggtcgagggcagcgggaaaacgggtcgataccctctcgaacagagtatgaatcgtctgagtcatcttttggcaacggcagtggcagtagaaaataccagcacagatatgatggaagttcacgtgacgcgccagcagcattatttcactgcgcgactgtcagatatttatcagtatcccggattggatattgccttacagccggatgaccgtatcactctgcgtcaagtgaccgagtatgttaatgtgctcggcgcagcaggtgttcaggggaaacatgctctggttcagcgtcattccagcgtcgtggatgctttggcgctcgcgaaaggattaaatgacaatcttgccgatccgcaagcgatttttctgtacaagcataacgaagcggaacaggcgaaacagcagatgcgtaagctgaatatctatcatgtcgatatgagccaacctaactcggtgtttttagcgcaggccatacgagtggacaacggcgatgttatctatatctcgaacgcctctttgactgatttcgctaaggtgaaagccgcattcgatagctttttgac

SEQ ID NO: 8 - DNA, 16278 bp, Artificial Sequence
  Ty21a-eS-AR Construct
  misc_feature (3573)..(3573): n is a, c, g, or t
cccgttcctcattgatttgattgctaacgtcatgagcattactctatcattccagaatgcctcaatgaacaagttgtttgagaaagagtgtcgcaatgttgcaaccagagcccttaaatatgtacgccagaagaaaactgaagggcgtctggatgaagcattgtctgtattgattagcctgaaacgaattgagcctgatgtttctcgtctgatgcgtgaatataagcaaattatcagattatttaatgagtcacggaaggatggcggtagcactatcacgtcttatgaacatctagactatgcgaaaaaattactcgtttttgatagcgaaaatgcctatgccttgaaatatgccgcattaaatgcaatgcatttacgcgactacacgcaggctttgcagtattggcagcgactggagaaagtgaatggaccaacggagccggtgacaaggcagatctcgacctgcataaccgcattacaaaaaaatacatcagggaagtcgtaacccggggtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatatgcattaatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttgcgatcgttatgacaacttgacggctacatcattcactttttcttcacaaccggcacggaactcgctcgggctggccccggtgcattttttaaatacccgcgagaagtagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaataacacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctcggagttaacaaaagatgttagatgcaaacaaattacagcaggcagtggatcaggcttacacccaatttcactcacttaacggcggacaaaatgccgattacattccctttctggcgaatgtaccaggtcaactggcggcagtggctatcgtgacntgcgatggcaacgtctatagtgcgggtgacagtgattaccgctttgcactggaatccatctctaaagtctgtacgttagcccttgcgttagaagatgtcggcccgcaggcggtacaggacaaaattggcgctgacccgaccggattgccctttaactcagttatcgccttagagttgcatggcggcaaaccgctgtcgccactggtaaatgctggcgctattgccaccaccagcctgattaacgctgaaaatgttgaacaacgctggcagcgaattttacatatccaacagcaactggctggtgagcaggtagcgctctctgacgaagtcaaccagtcggaacaaacaaccaacttccataaccgggccattgcctggctgctgtactccgccggatatctctattgtgatgcaatggaagcctgtgacgtgtacacccgtcagtgctctacgctcatcaatactgttgaactggcaacgcttggcgcgacgctggcggcgggtggtgtgaatccgttgacgcataaacgcgttcttcaggccgacaacgtgccgtacattctggccgaaatgatgatggaagggctgtatggtcgctccggtgactgggcgtatcgcgttggtttaccgggcaaaagcggtgtaggtggcggtattctggcggtcgtccctggcgtgatgggaattgccgcgttctcaccaccgctggacgaagaaggcaacagtgttcgcggtcaaaaaatggtggcatcggtcgctaagcaactcggctataacgtgtttaagggctgaccatggagtgttttaatgcgatccaatcattttaaggagtttaaaatggataagaagcaagtaacggatttaaggtcggaactactcgattcacgttttggtgcgaagtctatttccactatcgcagaatcaaaacgttttccgctgcacgaaatgcgcgacgatgtcgcatttcagattatcaatgatgaattatatcttgatggcaacgctcgtcagaacctggccactttctgccagacctgggacgacgaaaacgtccacaagttgatggatttatccattaacaaaaactggatcgacaaagaagaatatccgcaatccgcagccatcgacctgcgttgcgtaaacatggttgccgatctgtggcatgcgcctgcgccgaaaaatggtcaggccgttggcaccaacaccattggttcttccgaggcctgtatgctcggcgggatggcgatgaaatggcgttggcgcaagcgtatggaagctgcaggcaaaccaacggataaaccaaacctggtgtgcggtccggtgcaaatctgctggcataaattcgcccgctactgggatgtggagctgcgtgagatccctatgcgccccggtcagttgtttatggacccgaaacgcatgattgaagcctgcgacgaaaataccatcggcgtggtgccgactttcggcgtgacctacaccggtaactatgagttcccgcaaccgctgcacgatgcactggataaattccaggccgacaccggtatcgacatcgacatgcacatcgacgccgccagcggtggctttctggcaccgttcgtcgccccggatatcgtctgggacttccgcctgccgcgtgtgaaatcgatcagtgcttcaggccataaattcggtctggctccgctgggctgcggctgggttatctggcgtgacgaagaagcgctgccgcaggaactggtgttcaacgttgactacctcggtggtcagattggtacttttgccatcaacttctcccgcccggcgggtcaggtgattgcacagtactatgaattcctgcgccttggtcgtgaaggctataccaaagtacagaatgcttcctaccaggtcgctgcctatctggcggatgagatcgccaaactggggccgtatgagttcatctgtaccggtcgcccggacgaaggcatcccggcggtttgcttcaaactgaaagatggtgaagatccgggatacaccctgtacgacctctctgaacgtctgcgtctgcgcggctggcaggttccggccttcactctcggcggtgaagccaccgacatcgtggtgatgcgcattatgtgtcgtcgcggcttcgaaatggactttgctgaactgttgctggaagactacaaagcctccctgaaatatctcagcgatcacccgaaactgcagggtattgcccagcagaacagctttaaacatacctgataaccgttttggagtgataatatggctacatcagtacagacaggtaaagctaagcagctcacattactcggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctgggtccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttacctgcactccggtgcccccgttgctatcgaaatggattcgaagaccttcttccctgacttctctaaagtgggcacactggttgtatttgttgccttcattttgagttatatgggcgtagaagcttccgcaactcacgtcaatgaaatgagcaaccccgggcgcgactatccactggctatgttactgctgatggtggcggcaatctgcttaagctctgttggcggtttgtctattgcgatggtcattccgggtaatgaaatcaacctctccgcaggggtaatgcaaacctttaccgttctgatgtcccatgtggcaccggaaattgagtggacggttcgcgtgatctccgcactgctgttgctgggtgttctggcggaaatcgcctcctggattgttggtccttctcgcgggatgtatgtcacagcgcagaaaaacctgctgccagcggcattcgctaaaatgaacaaaaatggcgtaccggtaacgctggtcatttcgcagctggtgattacttctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaaggcaaagcgaataccggcgtcactctggagccaatcaacagtcagaacgcaccaaaaggtcacttcttcctgcacccgcgtgcacgttcaccacactatattgtgatgaatgacaagaaacactaacgatcggcgaagtaatcgcaacatccgcattaaaatctagcgagggctttactgtcgaccaaaaaaatattgacaacataaaaaactttgtgttatacttgtaacggactctagagggtattaataatgaaaggagaggacatgagctcatgtttgtgtttctggtgctgctgccgctggtgagcagccagtgcgtgaacctgaccacccgcacccagctgccgccggcgtataccaacagctttacccgcggcgtgtattatccggataaagtgtttcgcagcagcgtgctgcatagcacccaggatctgtttctgccgttttttagcaacgtgacctggtttcatgcgattcatgtgagcggcaccaacggcaccaaacgctttgataacccggtgctgccgtttaacgatggcgtgtattttgcgagcaccgaaaaaagcaacattattcgcggctggatttttggcaccaccctggatagcaaaacccagagcctgctgattgtgaacaacgcgaccaacgtggtgattaaagtgtgcgaatttcagttttgcaacgatccgtttctgggcgtgtattatcataaaaacaacaaaagctggatggaaagcgaatttcgcgtgtatagcagcgcgaacaactgcacctttgaatatgtgagccagccgtttctgatggatctggaaggcaaacagggcaactttaaaaacctgcgcgaatttgtgtttaaaaacattgatggctattttaaaatttatagcaaacataccccgattaacctggtgcgcgatctgccgcagggctttagcgcgctggaaccgctggtggatctgccgattggcattaacattacccgctttcagaccctgctggcgctgcatcgcagctatctgaccccgggcgatagcagcagcggctggaccgcgggcgcggcggcgtattatgtgggctatctgcagccgcgcacctttctgctgaaatataacgaaaacggcaccattaccgatgcggtggattgcgcgctggatccgctgagcgaaaccaaatgcaccctgaaaagctttaccgtggaaaaaggcatttatcagaccagcaactttcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaactttaactttaacggcctgaccggcaccggcgtgctgaccgaaagcaacaaaaaatttctgccgtttcagcagtttggccgcgatattgcggataccaccgatgcggtgcgcgatccgcagaccctggaaattctggatattaccccgtgcagctttggcggcgtgagcgtgattaccccgggcaccaacaccagcaaccaggtggcggtgctgtatcagggcgtgaactgcaccgaagtgccggtggcgattcatgcggatcagctgaccccgacctggcgcgtgtatagcaccggcagcaacgtgtttcagacccgcgcgggctgcctgattggcgcggaacatgtgaacaacagctatgaatgcgatattccgattggcgcgggcatttgcgcgagctatcagacccagaccaacagcccgggcagcgcgagcagcgtggcgagccagagcattattgcgtataccatgagcctgggcgcggaaaacagcgtggcgtatagcaacaacagcattgcgattccgaccaactttaccattagcgtgaccaccgaaattctgccggtgagcatgaccaaaaccagcgtggattgcaccatgtatatttgcggcgatagcaccgaatgcagcaacctgctgctgcagtatggcagcttttgcacccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcgggcgcggcgctgcagattccgtttgcgatgcagatggcgtatcgctttaacggcattggcgtgacccagaacgtgctgtatgaaaaccagaaactgattgcgaaccagtttaacagcgcgattggcaaaattcaggatagcctgagcagcaccgcgagcgcgctgggcaaactgcaggatgtggtgaaccagaacgcgcaggcgctgaacaccctggtgaaacagctgagcagcaactttggcgcgattagcagcgtgctgaacgatattctgagccgcctggatccgccggaagcggaagtgcagattgatcgcctgattaccggccgcctgcagagcctgcagacctatgtgacccagcagctgattcgcgcggcggaaattcgcgcgagcgcgaacctggcggcgaccaaaatgagcgaatgcgtgctgggccagagcaaacgcgtggatttttgcggcaaaggctatcatctgatgagctttccgcagagcgcgccgcatggcgtggtgtttctgcatgtgacctatgtgccggcgcaggaaaaaaactttaccaccgcgccggcgatttgccatgatggcaaagcgcattttccgcgcgaaggcgtgtttgtgagcaacggcacccattggtttgtgacccagcgcaacttttatgaaccgcagattattaccaccgataacacctttgtgagcggcaactgcgatgtggtgattggcattgtgaacaacaccgtgtatgatccgctgcagccggaactggatagctttaaagaagaactggataaatattttaaaaaccataccagcccggatgtggatctgggcgatattagcggcattaacgcgagcgtggtgaacattcagaaagaaattgatcgcctgaacgaagtggcgaaaaacctgaacgaaagcctgattgatctgcaggaactgggcaaatatgaacagggctatattccggaagcgccgcgcgatggccaggcgtatgtgcgcaaagatggcgaatgggtgctgctgagcacctttctggcattagcctatggaagtcagggtaatcttaatccattaattaatgaaatcagcaaaatcatttcagctgcaggtaattttgatgttaaagaggaaagagcggccgcgtctttattgcagttgtccggtaatgccagtgatttttcatatggacggaactcaataactttgacagcatcagcataagagctctttattaatttaaataatagcaatcttactgggctgtgccacataagattgctatttttttggagtcataatggattcttgtcataaaattgattatgggttatacgccctggagattttagcccaataccataacgtctctgttaacccggaagaaattaaacatagatttgacacagacgggactggtctgggattaacgtcatggttgcttgctgcgaaatctttagaactaaaggtaaaacaggtaaaaaaaacaattgaccgattaaactttatttctctgcccgcattagtctggagagaggatggatgtcattttattctgactaaagtcagtaaagaagcaaacagatatcttatttttgatctggagcagcgaaatccccgtgttctcgaacagtctgagtttgaggcgttatatcaggggcatattattcttattgcttcccgttcttctgttaccgggaaactggcaaaatttgactttacctggtttattcctgccattataaaatacaggaaaatatttattgaaacccttgttgtatctgtttttttacaattatttgcattaataaccccccttttttttcaggtggttatggacaaagtattagtacacagggggttttcaacccttaatgttattactgtcgcattatctgttgtggtggtgtttgagattatactcagcggtttaagaacttacatttttgcacatagtacaagtcggattgatgttgagttgggtgccaaactcttccggcatttactggcgctaccgatctcttattttgagagtcgtcgtgttggtgatactgttgccagggtaagagaattagaccagatccgtaatttcctgacaggacaggcattaacatctgttctggacttattattttcattcatattttttgcggtaatgtggtattacagcccaaagcttactctggtgatcttattttcgctgccctgttatgctgcatggtctgtttttattagccccattttgcgacgtcgccttgatgataagttttcacggaatgcggataatcaatctttcctggtggaatcagtcacggcgattaacactataaaagctatggcagtctcacctcagatgacgaacatatgggacaaacaattggcaggatatgttgctgcaggctttaaagtgacagtattagccaccattggtcaacaaggaatacagttaatacaaaagactgttatgatcatcaacctgtggttgggtgcacacctggttatttccggggatttaagtattggtcagttaattgcttttaatatgcttgcaggtcagattgttgcaccggttattcgccttgcacaaatctggcaggatttccagcaggttggtatatcagttacccgccttggtgatgtgcttaactctccaactgaaagttatcatgggaaactggcattaccggaaattaatggtgatatcacttttcgtaatatccggtttcgctataagcctgactctccggttattttagataatatcaatctcagtattaagcagggggaggttattggtattgtcggacgttctggttcaggaaaaagcacattaactaaattaattcaacgtttttatattcctgaaaatggccaggtcttaattgatggacatgatcttgcgttggccgatcctaactggttacgtcgtcaggtgggggttgtgttgcaggacaatgtgctgcttaatcgcagtattattgataatatttcactggctaatcctggcatgtccgtcgaaaaagttatttatgcagcgaaattagcaggtgctcatgattttatttctgaattgcgtgaggggtataacaccattgtcggggaacagggggcaggattatccggaggtcaacgtcaacgcatcgcaattgcaagggcgctggtgaacaaccctaaaatactcatctttgatgaagcaaccagtgctctggattatgagtcggagcatgtcatcatgcgcaatatgcacaaaatatgtaagggcagaacggttataatcattgctcatcgtctgtctacagtaaaaaatgcagaccgcattattgtcatggaaaaagggaaaattgttgaacagggtaaacataaggagctgctttctgaaccggaaagtttatacagttacttatatcagttacagtcagactaacagaaagaacagaagaatatgaaaacatggttaatggggttcagcgagttcctgttgcgctataaacttgtctggagtgaaacatggaaaatccggaagcagttagatactccggtacgtgaaaaggacgaaaatgaattcttacccgctcatctggaattaattgaaacgccggtatccagacggccgcgtctggttgcttattttattatggggtttctggttattgctttcattttatctgttttaggccaggtggaaattgttgccactgcaaatgggaaattaacactcagtgggcgtagcaaagaaattaaacctattgaaaactcgatagttaaagaaattatcgtaaaagaaggagagtcagtccggaaaggggatgtgttattaaagcttacagcgctgggagctgaagctgatacgttaaaaacgcagtcatcactgttacaggccaggctggaacaaattcggtatcaaattctgagccggtcaattgaattaaataaacttcctgaactgaaacttcctgatgagccttattttcagaatgtatctgaagaggaagtactgcgtttaacttctttgataaaagaacagttttccacatggcaaaatcagaagtatcaaaaagaactgaatctggataagaaaagagcagagcgattaacaatacttgcccgtataaaccgttatgaaaatgtatcgagggttgaaaaaagccgtctggatgatttcaggagcctgttgcataaacaggcaattgcaaaacatgctgtacttgagcaggagaataaatatgttgaggcagcaaatgaattacgggtttataaatcgcaactggagcaaattgagagtgagatattgtctgcaaaagaagaatatcagcttgtcacgcagctttttaaaaatgaaattttagacaagctaagacaaacaacagacagcattgagttattaactctggagttagagaaaaatgaagagcgtcaacaggcttcagtaatcagggcccctgtttcgggaaaagttcagcaactgaaggttcatactgaaggtggggttgttacaacagcggaaacactgatggtcatcgttccggaagatgacacgctggaggttactgctctggtacaaaataaagatattggttttattaacgtcgggcagaatgccatcattaaagtggaggcttttccttacacccgatatggttatctggtgggtaaggtaaaaaatataaatttagatgcaatagaagaccagaaactgggactcgtttttaatgtcattgtttctgttgaagagaatgatttgtcaaccgggaataagcacattccattaagctcgggtatggctgtcactgcagaaataaagactggaatgcgaagcgtaatcagttatcttcttagtcccctggaagaatctgtaacagaaagtttacatgagcgttaagtctcagagcagcggtatccggctcatatcttctcctgtcagatccaagggcgaattccagcacactggcggccgttactagtggatccaccggtctcgaggggcgcgccggtaccgaattctaattaatgggcatcatttttcagctatttcatttataaaataagttatgaaaaaaatcatcatattactaacgacatttttcctgctttcgggatgcactattcccagggcggtatttaaatccagccttattaatcaggacgatcctcgttataatctggtcgaagtcacgccgacattaaaactaagcgctcccgatactgtgccgaaaactattgtcgatccggtttttgccgcaaataactggcactggacatctttggctaaaggcgatgtgctgcatatcactattttatcctcgggcggggctggatatttatccaataacgcgagcggcgaccgtgcggattttgaaaatattcttgtgactgacagtaataccgttcaggtgccttatgccgggacaatcccggtttctggattggatgtgacgcaactggctgatgagatcaaaaagcgactttcgcgcgttgtcctgaatcctcaggtgattgtgacacttaccgcccgcaccggagccatggtgacggtcgagggcagcgggaaaacgggtcgataccctctcgaacagagtatgaatcgtctgagtcatcttttggcaacggcagtggcagtagaaaataccagcacagatatgatggaagttcacgtgacgcgccagcagcattatttcactgcgcgactgtcagatatttatcagtatcccggattggatattgccttacagccggatgaccgtatcactctgcgtcaagtgaccgagtatgttaatgtgctcggcgcagcaggtgttcaggggaaacatgctctggttcagcgtcattccagcgtcgtggatgctttggcgctcgcgaaaggattaaatgacaatcttgccgatccgcaagcgatttttctgtacaagcataacgaagcggaacaggcgaaacagcagatgcgtaagctgaatatctatcatgtcgatatgagccaacctaactcggtgtttttagcgcaggccatacgagtggacaacggcgatgttatctatatctcgaacgcctctttgactgatttcgctaaggtgaaagccgcattcgatagctttttgac

SEQ ID NO: 9 - DNA, 14520 bp, Artificial Sequence
  Ty21a-M-N-AR Construct
  misc_feature (3573)..(3573): n is a, c, g, or t
cccgttcctcattgatttgattgctaacgtcatgagcattactctatcattccagaatgcctcaatgaacaagttgtttgagaaagagtgtcgcaatgttgcaaccagagcccttaaatatgtacgccagaagaaaactgaagggcgtctggatgaagcattgtctgtattgattagcctgaaacgaattgagcctgatgtttctcgtctgatgcgtgaatataagcaaattatcagattatttaatgagtcacggaaggatggcggtagcactatcacgtcttatgaacatctagactatgcgaaaaaattactcgtttttgatagcgaaaatgcctatgccttgaaatatgccgcattaaatgcaatgcatttacgcgactacacgcaggctttgcagtattggcagcgactggagaaagtgaatggaccaacggagccggtgacaaggcagatctcgacctgcataaccgcattacaaaaaaatacatcagggaagtcgtaacccggggtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatatgcattaatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttgcgatcgttatgacaacttgacggctacatcattcactttttcttcacaaccggcacggaactcgctcgggctggccccggtgcattttttaaatacccgcgagaagtagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaataacacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctcggagttaacaaaagatgttagatgcaaacaaattacagcaggcagtggatcaggcttacacccaatttcactcacttaacggcggacaaaatgccgattacattccctttctggcgaatgtaccaggtcaactggcggcagtggctatcgtgacntgcgatggcaacgtctatagtgcgggtgacagtgattaccgctttgcactggaatccatctctaaagtctgtacgttagcccttgcgttagaagatgtcggcccgcaggcggtacaggacaaaattggcgctgacccgaccggattgccctttaactcagttatcgccttagagttgcatggcggcaaaccgctgtcgccactggtaaatgctggcgctattgccaccaccagcctgattaacgctgaaaatgttgaacaacgctggcagcgaattttacatatccaacagcaactggctggtgagcaggtagcgctctctgacgaagtcaaccagtcggaacaaacaaccaacttccataaccgggccattgcctggctgctgtactccgccggatatctctattgtgatgcaatggaagcctgtgacgtgtacacccgtcagtgctctacgctcatcaatactgttgaactggcaacgcttggcgcgacgctggcggcgggtggtgtgaatccgttgacgcataaacgcgttcttcaggccgacaacgtgccgtacattctggccgaaatgatgatggaagggctgtatggtcgctccggtgactgggcgtatcgcgttggtttaccgggcaaaagcggtgtaggtggcggtattctggcggtcgtccctggcgtgatgggaattgccgcgttctcaccaccgctggacgaagaaggcaacagtgttcgcggtcaaaaaatggtggcatcggtcgctaagcaactcggctataacgtgtttaagggctgaccatggagtgttttaatgcgatccaatcattttaaggagtttaaaatggataagaagcaagtaacggatttaaggtcggaactactcgattcacgttttggtgcgaagtctatttccactatcgcagaatcaaaacgttttccgctgcacgaaatgcgcgacgatgtcgcatttcagattatcaatgatgaattatatcttgatggcaacgctcgtcagaacctggccactttctgccagacctgggacgacgaaaacgtccacaagttgatggatttatccattaacaaaaactggatcgacaaagaagaatatccgcaatccgcagccatcgacctgcgttgcgtaaacatggttgccgatctgtggcatgcgcctgcgccgaaaaatggtcaggccgttggcaccaacaccattggttcttccgaggcctgtatgctcggcgggatggcgatgaaatggcgttggcgcaagcgtatggaagctgcaggcaaaccaacggataaaccaaacctggtgtgcggtccggtgcaaatctgctggcataaattcgcccgctactgggatgtggagctgcgtgagatccctatgcgccccggtcagttgtttatggacccgaaacgcatgattgaagcctgcgacgaaaataccatcggcgtggtgccgactttcggcgtgacctacaccggtaactatgagttcccgcaaccgctgcacgatgcactggataaattccaggccgacaccggtatcgacatcgacatgcacatcgacgccgccagcggtggctttctggcaccgttcgtcgccccggatatcgtctgggacttccgcctgccgcgtgtgaaatcgatcagtgcttcaggccataaattcggtctggctccgctgggctgcggctgggttatctggcgtgacgaagaagcgctgccgcaggaactggtgttcaacgttgactacctcggtggtcagattggtacttttgccatcaacttctcccgcccggcgggtcaggtgattgcacagtactatgaattcctgcgccttggtcgtgaaggctataccaaagtacagaatgcttcctaccaggtcgctgcctatctggcggatgagatcgccaaactggggccgtatgagttcatctgtaccggtcgcccggacgaaggcatcccggcggtttgcttcaaactgaaagatggtgaagatccgggatacaccctgtacgacctctctgaacgtctgcgtctgcgcggctggcaggttccggccttcactctcggcggtgaagccaccgacatcgtggtgatgcgcattatgtgtcgtcgcggcttcgaaatggactttgctgaactgttgctggaagactacaaagcctccctgaaatatctcagcgatcacccgaaactgcagggtattgcccagcagaacagctttaaacatacctgataaccgttttggagtgataatatggctacatcagtacagacaggtaaagctaagcagctcacattactcggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctgggtccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttacctgcactccggtgcccccgttgctatcgaaatggattcgaagaccttcttccctgacttctctaaagtgggcacactggttgtatttgttgccttcattttgagttatatgggcgtagaagcttccgcaactcacgtcaatgaaatgagcaaccccgggcgcgactatccactggctatgttactgctgatggtggcggcaatctgcttaagctctgttggcggtttgtctattgcgatggtcattccgggtaatgaaatcaacctctccgcaggggtaatgcaaacctttaccgttctgatgtcccatgtggcaccggaaattgagtggacggttcgcgtgatctccgcactgctgttgctgggtgttctggcggaaatcgcctcctggattgttggtccttctcgcgggatgtatgtcacagcgcagaaaaacctgctgccagcggcattcgctaaaatgaacaaaaatggcgtaccggtaacgctggtcatttcgcagctggtgattacttctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaaggcaaagcgaataccggcgtcactctggagccaatcaacagtcagaacgcaccaaaaggtcacttcttcctgcacccgcgtgcacgttcaccacactatattgtgatgaatgacaagaaacactaacgatcggcgaagtaatcgcaacatccgcattaaaatctagcgagggctttactgtcgaccaaaaaaatattgacaacataaaaaactttgtgttatacttgtaacggactctagagggtattaataatgaaaggagaggacatgagctccctaggatgagcgataacggcccgcagaaccagcgcaacgcgccgcgcattacctttggcggcccgagcgatagcaccggcagcaaccagaacggcgaacgcagcggcgcgcgcagcaaacagcgccgcccgcagggcctgccgaacaacaccgcgagctggtttaccgcgctgacccagcatggcaaagaagatctgaaatttccgcgcggccagggcgtgccgattaacaccaacagcagcccggatgatcagattggctattatcgccgcgcgacccgccgcattcgcggcggcgatggcaaaatgaaagatctgagcccgcgctggtatttttattatctgggcaccggcccggaagcgggcctgccgtatggcgcgaacaaagatggcattatttgggtggcgaccgaaggcgcgctgaacaccccgaaagatcatattggcacccgcaacccggcgaacaacgcggcgattgtgctgcagctgccgcagggcaccaccctgccgaaaggcttttatgcggaaggcagccgcggcggcagccaggcgagcagccgcagcagcagccgcagccgcaacagcagccgcaacagcaccccgggcagcagccgcggcaccagcccggcgcgcatggcgggcaacggcggcgatgcggcgctggcgctgctgctgctggatcgcctgaaccagctggaaagcaaaatgagcggcaaaggccagcagcagcagggccagaccgtgaccaaaaaaagcgcggcggaagcgagcaaaaaaccgcgccagaaacgcaccgcgaccaaagcgtataacgtgacccaggcgtttggccgccgcggcccggaacagacccagggcaactttggcgatcaggaactgattcgccagggcaccgattataaacattggccgcagattgcgcagtttgcgccgagcgcgagcgcgttttttggcatgagccgcattggcatggaagtgaccccgagcggcacctggctgacctataccggcgcgattaaactggatgataaagatccgaactttaaagatcaggtgattctgctgaacaaacatattgatgcgtataaaacctttccgccgaccgaaccgaaaaaagataaaaaaaaaaaagcggatgaaacccaggcgctgccgcagcgccagaaaaaacagcagaccgtgaccctgctgccggcggcggatctggatgattttagcaaacagctgcagcagagcatgagcagcgcggatagcacccaggcgcctaggcttaagatggcggatagcaacggcaccattaccgtggaagaactgaaaaaactgctggaacagtggaacctggtgattggctttctgtttctgacctggatttgcctgctgcagtttgcgtatgcgaaccgcaaccgctttctgtatattattaaactgatttttctgtggctgctgtggccggtgaccctggcgtgctttgtgctggcggcggtgtatcgcattaactggattaccggcggcattgcgattgcgatggcgtgcctggtgggcctgatgtggctgagctattttattgcgagctttcgcctgtttgcgcgcacccgcagcatgtggagctttaacccggaaaccaacattctgctgaacgtgccgctgcatggcaccattctgacccgcccgctgctggaaagcgaactggtgattggcgcggtgattctgcgcggccatctgcgcattgcgggccatcatctgggccgctgcgatattaaagatctgccgaaagaaattaccgtggcgaccagccgcaccctgagctattataaactgggcgcgagccagcgcgtggcgggcgatagcggctttgcggcgtatagccgctatcgcattggcaactataaactgaacaccgatcatagcagcagcagcgataacattgcgctgctggtgcagcttaaggcattagcctatggaagtcagggtaatcttaatccattaattaatgaaatcagcaaaatcatttcagctgcaggtaattttgatgttaaagaggaaagagcggccgcgtctttattgcagttgtccggtaatgccagtgatttttcatatggacggaactcaataactttgacagcatcagcataagagctctttattaatttaaataatagcaatcttactgggctgtgccacataagattgctatttttttggagtcataatggattcttgtcataaaattgattatgggttatacgccctggagattttagcccaataccataacgtctctgttaacccggaagaaattaaacatagatttgacacagacgggactggtctgggattaacgtcatggttgcttgctgcgaaatctttagaactaaaggtaaaacaggtaaaaaaaacaattgaccgattaaactttatttctctgcccgcattagtctggagagaggatggatgtcattttattctgactaaagtcagtaaagaagcaaacagatatcttatttttgatctggagcagcgaaatccccgtgttctcgaacagtctgagtttgaggcgttatatcaggggcatattattcttattgcttcccgttcttctgttaccgggaaactggcaaaatttgactttacctggtttattcctgccattataaaatacaggaaaatatttattgaaacccttgttgtatctgtttttttacaattatttgcattaataaccccccttttttttcaggtggttatggacaaagtattagtacacagggggttttcaacccttaatgttattactgtcgcattatctgttgtggtggtgtttgagattatactcagcggtttaagaacttacatttttgcacatagtacaagtcggattgatgttgagttgggtgccaaactcttccggcatttactggcgctaccgatctcttattttgagagtcgtcgtgttggtgatactgttgccagggtaagagaattagaccagatccgtaatttcctgacaggacaggcattaacatctgttctggacttattattttcattcatattttttgcggtaatgtggtattacagcccaaagcttactctggtgatcttattttcgctgccctgttatgctgcatggtctgtttttattagccccattttgcgacgtcgccttgatgataagttttcacggaatgcggataatcaatctttcctggtggaatcagtcacggcgattaacactataaaagctatggcagtctcacctcagatgacgaacatatgggacaaacaattggcaggatatgttgctgcaggctttaaagtgacagtattagccaccattggtcaacaaggaatacagttaatacaaaagactgttatgatcatcaacctgtggttgggtgcacacctggttatttccggggatttaagtattggtcagttaattgcttttaatatgcttgcaggtcagattgttgcaccggttattcgccttgcacaaatctggcaggatttccagcaggttggtatatcagttacccgccttggtgatgtgcttaactctccaactgaaagttatcatgggaaactggcattaccggaaattaatggtgatatcacttttcgtaatatccggtttcgctataagcctgactctccggttattttagataatatcaatctcagtattaagcagggggaggttattggtattgtcggacgttctggttcaggaaaaagcacattaactaaattaattcaacgtttttatattcctgaaaatggccaggtcttaattgatggacatgatcttgcgttggccgatcctaactggttacgtcgtcaggtgggggttgtgttgcaggacaatgtgctgcttaatcgcagtattattgataatatttcactggctaatcctggcatgtccgtcgaaaaagttatttatgcagcgaaattagcaggtgctcatgattttatttctgaattgcgtgaggggtataacaccattgtcggggaacagggggcaggattatccggaggtcaacgtcaacgcatcgcaattgcaagggcgctggtgaacaaccctaaaatactcatctttgatgaagcaaccagtgctctggattatgagtcggagcatgtcatcatgcgcaatatgcacaaaatatgtaagggcagaacggttataatcattgctcatcgtctgtctacagtaaaaaatgcagaccgcattattgtcatggaaaaagggaaaattgttgaacagggtaaacataaggagctgctttctgaaccggaaagtttatacagttacttatatcagttacagtcagactaacagaaagaacagaagaatatgaaaacatggttaatggggttcagcgagttcctgttgcgctataaacttgtctggagtgaaacatggaaaatccggaagcagttagatactccggtacgtgaaaaggacgaaaatgaattcttacccgctcatctggaattaattgaaacgccggtatccagacggccgcgtctggttgcttattttattatggggtttctggttattgctttcattttatctgttttaggccaggtggaaattgttgccactgcaaatgggaaattaacactcagtgggcgtagcaaagaaattaaacctattgaaaactcgatagttaaagaaattatcgtaaaagaaggagagtcagtccggaaaggggatgtgttattaaagcttacagcgctgggagctgaagctgatacgttaaaaacgcagtcatcactgttacaggccaggctggaacaaattcggtatcaaattctgagccggtcaattgaattaaataaacttcctgaactgaaacttcctgatgagccttattttcagaatgtatctgaagaggaagtactgcgtttaacttctttgataaaagaacagttttccacatggcaaaatcagaagtatcaaaaagaactgaatctggataagaaaagagcagagcgattaacaatacttgcccgtataaaccgttatgaaaatgtatcgagggttgaaaaaagccgtctggatgatttcaggagcctgttgcataaacaggcaattgcaaaacatgctgtacttgagcaggagaataaatatgttgaggcagcaaatgaattacgggtttataaatcgcaactggagcaaattgagagtgagatattgtctgcaaaagaagaatatcagcttgtcacgcagctttttaaaaatgaaattttagacaagctaagacaaacaacagacagcattgagttattaactctggagttagagaaaaatgaagagcgtcaacaggcttcagtaatcagggcccctgtttcgggaaaagttcagcaactgaaggttcatactgaaggtggggttgttacaacagcggaaacactgatggtcatcgttccggaagatgacacgctggaggttactgctctggtacaaaataaagatattggttttattaacgtcgggcagaatgccatcattaaagtggaggcttttccttacacccgatatggttatctggtgggtaaggtaaaaaatataaatttagatgcaatagaagaccagaaactgggactcgtttttaatgtcattgtttctgttgaagagaatgatttgtcaaccgggaataagcacattccattaagctcgggtatggctgtcactgcagaaataaagactggaatgcgaagcgtaatcagttatcttcttagtcccctggaagaatctgtaacagaaagtttacatgagcgttaagtctcagagcagcggtatccggctcatatcttctcctgtcagatccaagggcgaattccagcacactggcggccgttactagtggatccaccggtctcgaggggcgcgccggtaccgaattctaattaatgggcatcatttttcagctatttcatttataaaataagttatgaaaaaaatcatcatattactaacgacatttttcctgctttcgggatgcactattcccagggcggtatttaaatccagccttattaatcaggacgatcctcgttataatctggtcgaagtcacgccgacattaaaactaagcgctcccgatactgtgccgaaaactattgtcgatccggtttttgccgcaaataactggcactggacatctttggctaaaggcgatgtgctgcatatcactattttatcctcgggcggggctggatatttatccaataacgcgagcggcgaccgtgcggattttgaaaatattcttgtgactgacagtaataccgttcaggtgccttatgccgggacaatcccggtttctggattggatgtgacgcaactggctgatgagatcaaaaagcgactttcgcgcgttgtcctgaatcctcaggtgattgtgacacttaccgcccgcaccggagccatggtgacggtcgagggcagcgggaaaacgggtcgataccctctcgaacagagtatgaatcgtctgagtcatcttttggcaacggcagtggcagtagaaaataccagcacagatatgatggaagttcacgtgacgcgccagcagcattatttcactgcgcgactgtcagatatttatcagtatcccggattggatattgccttacagccggatgaccgtatcactctgcgtcaagtgaccgagtatgttaatgtgctcggcgcagcaggtgttcaggggaaacatgctctggttcagcgtcattccagcgtcgtggatgctttggcgctcgcgaaaggattaaatgacaatcttgccgatccgcaagcgatttttctgtacaagcataacgaagcggaacaggcgaaacagcagatgcgtaagctgaatatctatcatgtcgatatgagccaacctaactcggtgtttttagcgcaggccatacgagtggacaacggcgatgttatctatatctcgaacgcctctttgactgatttcgctaaggtgaaagccgcattcgatagctttttgac

SEQ ID NO: 10 - DNA, 18219 bp, Artificial Sequence
  Ty21a-eS-M-N-AR Construct
  misc_feature (3573)..(3573): n is a, c, g, or t
cccgttcctcattgatttgattgctaacgtcatgagcattactctatcattccagaatgcctcaatgaacaagttgtttgagaaagagtgtcgcaatgttgcaaccagagcccttaaatatgtacgccagaagaaaactgaagggcgtctggatgaagcattgtctgtattgattagcctgaaacgaattgagcctgatgtttctcgtctgatgcgtgaatataagcaaattatcagattatttaatgagtcacggaaggatggcggtagcactatcacgtcttatgaacatctagactatgcgaaaaaattactcgtttttgatagcgaaaatgcctatgccttgaaatatgccgcattaaatgcaatgcatttacgcgactacacgcaggctttgcagtattggcagcgactggagaaagtgaatggaccaacggagccggtgacaaggcagatctcgacctgcataaccgcattacaaaaaaatacatcagggaagtcgtaacccggggtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatatgcattaatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttgcgatcgttatgacaacttgacggctacatcattcactttttcttcacaaccggcacggaactcgctcgggctggccccggtgcattttttaaatacccgcgagaagtagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaataacacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctcggagttaacaaaagatgttagatgcaaacaaattacagcaggcagtggatcaggcttacacccaatttcactcacttaacggcggacaaaatgccgattacattccctttctggcgaatgtaccaggtcaactggcggcagtggctatcgtgacntgcgatggcaacgtctatagtgcgggtgacagtgattaccgctttgcactggaatccatctctaaagtctgtacgttagcccttgcgttagaagatgtcggcccgcaggcggtacaggacaaaattggcgctgacccgaccggattgccctttaactcagttatcgccttagagttgcatggcggcaaaccgctgtcgccactggtaaatgctggcgctattgccaccaccagcctgattaacgctgaaaatgttgaacaacgctggcagcgaattttacatatccaacagcaactggctggtgagcaggtagcgctctctgacgaagtcaaccagtcggaacaaacaaccaacttccataaccgggccattgcctggctgctgtactccgccggatatctctattgtgatgcaatggaagcctgtgacgtgtacacccgtcagtgctctacgctcatcaatactgttgaactggcaacgcttggcgcgacgctggcggcgggtggtgtgaatccgttgacgcataaacgcgttcttcaggccgacaacgtgccgtacattctggccgaaatgatgatggaagggctgtatggtcgctccggtgactgggcgtatcgcgttggtttaccgggcaaaagcggtgtaggtggcggtattctggcggtcgtccctggcgtgatgggaattgccgcgttctcaccaccgctggacgaagaaggcaacagtgttcgcggtcaaaaaatggtggcatcggtcgctaagcaactcggctataacgtgtttaagggctgaccatggagtgttttaatgcgatccaatcattttaaggagtttaaaatggataagaagcaagtaacggatttaaggtcggaactactcgattcacgttttggtgcgaagtctatttccactatcgcagaatcaaaacgttttccgctgcacgaaatgcgcgacgatgtcgcatttcagattatcaatgatgaattatatcttgatggcaacgctcgtcagaacctggccactttctgccagacctgggacgacgaaaacgtccacaagttgatggatttatccattaacaaaaactggatcgacaaagaagaatatccgcaatccgcagccatcgacctgcgttgcgtaaacatggttgccgatctgtggcatgcgcctgcgccgaaaaatggtcaggccgttggcaccaacaccattggttcttccgaggcctgtatgctcggcgggatggcgatgaaatggcgttggcgcaagcgtatggaagctgcaggcaaaccaacggataaaccaaacctggtgtgcggtccggtgcaaatctgctggcataaattcgcccgctactgggatgtggagctgcgtgagatccctatgcgccccggtcagttgtttatggacccgaaacgcatgattgaagcctgcgacgaaaataccatcggcgtggtgccgactttcggcgtgacctacaccggtaactatgagttcccgcaaccgctgcacgatgcactggataaattccaggccgacaccggtatcgacatcgacatgcacatcgacgccgccagcggtggctttctggcaccgttcgtcgccccggatatcgtctgggacttccgcctgccgcgtgtgaaatcgatcagtgcttcaggccataaattcggtctggctccgctgggctgcggctgggttatctggcgtgacgaagaagcgctgccgcaggaactggtgttcaacgttgactacctcggtggtcagattggtacttttgccatcaacttctcccgcccggcgggtcaggtgattgcacagtactatgaattcctgcgccttggtcgtgaaggctataccaaagtacagaatgcttcctaccaggtcgctgcctatctggcggatgagatcgccaaactggggccgtatgagttcatctgtaccggtcgcccggacgaaggcatcccggcggtttgcttcaaactgaaagatggtgaagatccgggatacaccctgtacgacctctctgaacgtctgcgtctgcgcggctggcaggttccggccttcactctcggcggtgaagccaccgacatcgtggtgatgcgcattatgtgtcgtcgcggcttcgaaatggactttgctgaactgttgctggaagactacaaagcctccctgaaatatctcagcgatcacccgaaactgcagggtattgcccagcagaacagctttaaacatacctgataaccgttttggagtgataatatggctacatcagtacagacaggtaaagctaagcagctcacattactcggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctgggtccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttacctgcactccggtgcccccgttgctatcgaaatggattcgaagaccttcttccctgacttctctaaagtgggcacactggttgtatttgttgccttcattttgagttatatgggcgtagaagcttccgcaactcacgtcaatgaaatgagcaaccccgggcgcgactatccactggctatgttactgctgatggtggcggcaatctgcttaagctctgttggcggtttgtctattgcgatggtcattccgggtaatgaaatcaacctctccgcaggggtaatgcaaacctttaccgttctgatgtcccatgtggcaccggaaattgagtggacggttcgcgtgatctccgcactgctgttgctgggtgttctggcggaaatcgcctcctggattgttggtccttctcgcgggatgtatgtcacagcgcagaaaaacctgctgccagcggcattcgctaaaatgaacaaaaatggcgtaccggtaacgctggtcatttcgcagctggtgattacttctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaaggcaaagcgaataccggcgtcactctggagccaatcaacagtcagaacgcaccaaaaggtcacttcttcctgcacccgcgtgcacgttcaccacactatattgtgatgaatgacaagaaacactaacgatcggcgaagtaatcgcaacatccgcattaaaatctagcgagggctttactgtcgaccaaaaaaatattgacaacataaaaaactttgtgttatacttgtaacggactctagagggtattaataatgaaaggagaggacatatgagcgataacggcccgcagaaccagcgcaacgcgccgcgcattacctttggcggcccgagcgatagcaccggcagcaaccagaacggcgaacgcagcggcgcgcgcagcaaacagcgccgcccgcagggcctgccgaacaacaccgcgagctggtttaccgcgctgacccagcatggcaaagaagatctgaaatttccgcgcggccagggcgtgccgattaacaccaacagcagcccggatgatcagattggctattatcgccgcgcgacccgccgcattcgcggcggcgatggcaaaatgaaagatctgagcccgcgctggtatttttattatctgggcaccggcccggaagcgggcctgccgtatggcgcgaacaaagatggcattatttgggtggcgaccgaaggcgcgctgaacaccccgaaagatcatattggcacccgcaacccggcgaacaacgcggcgattgtgctgcagctgccgcagggcaccaccctgccgaaaggcttttatgcggaaggcagccgcggcggcagccaggcgagcagccgcagcagcagccgcagccgcaacagcagccgcaacagcaccccgggcagcagccgcggcaccagcccggcgcgcatggcgggcaacggcggcgatgcggcgctggcgctgctgctgctggatcgcctgaaccagctggaaagcaaaatgagcggcaaaggccagcagcagcagggccagaccgtgaccaaaaaaagcgcggcggaagcgagcaaaaaaccgcgccagaaacgcaccgcgaccaaagcgtataacgtgacccaggcgtttggccgccgcggcccggaacagacccagggcaactttggcgatcaggaactgattcgccagggcaccgattataaacattggccgcagattgcgcagtttgcgccgagcgcgagcgcgttttttggcatgagccgcattggcatggaagtgaccccgagcggcacctggctgacctataccggcgcgattaaactggatgataaagatccgaactttaaagatcaggtgattctgctgaacaaacatattgatgcgtataaaacctttccgccgaccgaaccgaaaaaagataaaaaaaaaaaagcggatgaaacccaggcgctgccgcagcgccagaaaaaacagcagaccgtgaccctgctgccggcggcggatctggatgattttagcaaacagctgcagcagagcatgagcagcgcggatagcacccaggcgcctaggcttaagatggcggatagcaacggcaccattaccgtggaagaactgaaaaaactgctggaacagtggaacctggtgattggctttctgtttctgacctggatttgcctgctgcagtttgcgtatgcgaaccgcaaccgctttctgtatattattaaactgatttttctgtggctgctgtggccggtgaccctggcgtgctttgtgctggcggcggtgtatcgcattaactggattaccggcggcattgcgattgcgatggcgtgcctggtgggcctgatgtggctgagctattttattgcgagctttcgcctgtttgcgcgcacccgcagcatgtggagctttaacccggaaaccaacattctgctgaacgtgccgctgcatggcaccattctgacccgcccgctgctggaaagcgaactggtgattggcgcggtgattctgcgcggccatctgcgcattgcgggccatcatctgggccgctgcgatattaaagatctgccgaaagaaattaccgtggcgaccagccgcaccctgagctattataaactgggcgcgagccagcgcgtggcgggcgatagcggctttgcggcgtatagccgctatcgcattggcaactataaactgaacaccgatcatagcagcagcagcgataacattgcgctgctggtgcagcttaaggagctcatgtttgtgtttctggtgctgctgccgctggtgagcagccagtgcgtgaacctgaccacccgcacccagctgccgccggcgtataccaacagctttacccgcggcgtgtattatccggataaagtgtttcgcagcagcgtgctgcatagcacccaggatctgtttctgccgttttttagcaacgtgacctggtttcatgcgattcatgtgagcggcaccaacggcaccaaacgctttgataacccggtgctgccgtttaacgatggcgtgtattttgcgagcaccgaaaaaagcaacattattcgcggctggatttttggcaccaccctggatagcaaaacccagagcctgctgattgtgaacaacgcgaccaacgtggtgattaaagtgtgcgaatttcagttttgcaacgatccgtttctgggcgtgtattatcataaaaacaacaaaagctggatggaaagcgaatttcgcgtgtatagcagcgcgaacaactgcacctttgaatatgtgagccagccgtttctgatggatctggaaggcaaacagggcaactttaaaaacctgcgcgaatttgtgtttaaaaacattgatggctattttaaaatttatagcaaacataccccgattaacctggtgcgcgatctgccgcagggctttagcgcgctggaaccgctggtggatctgccgattggcattaacattacccgctttcagaccctgctggcgctgcatcgcagctatctgaccccgggcgatagcagcagcggctggaccgcgggcgcggcggcgtattatgtgggctatctgcagccgcgcacctttctgctgaaatataacgaaaacggcaccattaccgatgcggtggattgcgcgctggatccgctgagcgaaaccaaatgcaccctgaaaagctttaccgtggaaaaaggcatttatcagaccagcaactttcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaactttaactttaacggcctgaccggcaccggcgtgctgaccgaaagcaacaaaaaatttctgccgtttcagcagtttggccgcgatattgcggataccaccgatgcggtgcgcgatccgcagaccctggaaattctggatattaccccgtgcagctttggcggcgtgagcgtgattaccccgggcaccaacaccagcaaccaggtggcggtgctgtatcagggcgtgaactgcaccgaagtgccggtggcgattcatgcggatcagctgaccccgacctggcgcgtgtatagcaccggcagcaacgtgtttcagacccgcgcgggctgcctgattggcgcggaacatgtgaacaacagctatgaatgcgatattccgattggcgcgggcatttgcgcgagctatcagacccagaccaacagcccgggcagcgcgagcagcgtggcgagccagagcattattgcgtataccatgagcctgggcgcggaaaacagcgtggcgtatagcaacaacagcattgcgattccgaccaactttaccattagcgtgaccaccgaaattctgccggtgagcatgaccaaaaccagcgtggattgcaccatgtatatttgcggcgatagcaccgaatgcagcaacctgctgctgcagtatggcagcttttgcacccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcgggcgcggcgctgcagattccgtttgcgatgcagatggcgtatcgctttaacggcattggcgtgacccagaacgtgctgtatgaaaaccagaaactgattgcgaaccagtttaacagcgcgattggcaaaattcaggatagcctgagcagcaccgcgagcgcgctgggcaaactgcaggatgtggtgaaccagaacgcgcaggcgctgaacaccctggtgaaacagctgagcagcaactttggcgcgattagcagcgtgctgaacgatattctgagccgcctggatccgccggaagcggaagtgcagattgatcgcctgattaccggccgcctgcagagcctgcagacctatgtgacccagcagctgattcgcgcggcggaaattcgcgcgagcgcgaacctggcggcgaccaaaatgagcgaatgcgtgctgggccagagcaaacgcgtggatttttgcggcaaaggctatcatctgatgagctttccgcagagcgcgccgcatggcgtggtgtttctgcatgtgacctatgtgccggcgcaggaaaaaaactttaccaccgcgccggcgatttgccatgatggcaaagcgcattttccgcgcgaaggcgtgtttgtgagcaacggcacccattggtttgtgacccagcgcaacttttatgaaccgcagattattaccaccgataacacctttgtgagcggcaactgcgatgtggtgattggcattgtgaacaacaccgtgtatgatccgctgcagccggaactggatagctttaaagaagaactggataaatattttaaaaaccataccagcccggatgtggatctgggcgatattagcggcattaacgcgagcgtggtgaacattcagaaagaaattgatcgcctgaacgaagtggcgaaaaacctgaacgaaagcctgattgatctgcaggaactgggcaaatatgaacagggctatattccggaagcgccgcgcgatggccaggcgtatgtgcgcaaagatggcgaatgggtgctgctgagcacctttctggcattagcctatggaagtcagggtaatcttaatccattaattaatgaaatcagcaaaatcatttcagctgcaggtaattttgatgttaaagaggaaagagcggccgcgtctttattgcagttgtccggtaatgccagtgatttttcatatggacggaactcaataactttgacagcatcagcataagagctctttattaatttaaataatagcaatcttactgggctgtgccacataagattgctatttttttggagtcataatggattcttgtcataaaattgattatgggttatacgccctggagattttagcccaataccataacgtctctgttaacccggaagaaattaaacatagatttgacacagacgggactggtctgggattaacgtcatggttgcttgctgcgaaatctttagaactaaaggtaaaacaggtaaaaaaaacaattgaccgattaaactttatttctctgcccgcattagtctggagagaggatggatgtcattttattctgactaaagtcagtaaagaagcaaacagatatcttatttttgatctggagcagcgaaatccccgtgttctcgaacagtctgagtttgaggcgttatatcaggggcatattattcttattgcttcccgttcttctgttaccgggaaactggcaaaatttgactttacctggtttattcctgccattataaaatacaggaaaatatttattgaaacccttgttgtatctgtttttttacaattatttgcattaataaccccccttttttttcaggtggttatggacaaagtattagtacacagggggttttcaacccttaatgttattactgtcgcattatctgttgtggtggtgtttgagattatactcagcggtttaagaacttacatttttgcacatagtacaagtcggattgatgttgagttgggtgccaaactcttccggcatttactggcgctaccgatctcttattttgagagtcgtcgtgttggtgatactgttgccagggtaagagaattagaccagatccgtaatttcctgacaggacaggcattaacatctgttctggacttattattttcattcatattttttgcggtaatgtggtattacagcccaaagcttactctggtgatcttattttcgctgccctgttatgctgcatggtctgtttttattagccccattttgcgacgtcgccttgatgataagttttcacggaatgcggataatcaatctttcctggtggaatcagtcacggcgattaacactataaaagctatggcagtctcacctcagatgacgaacatatgggacaaacaattggcaggatatgttgctgcaggctttaaagtgacagtattagccaccattggtcaacaaggaatacagttaatacaaaagactgttatgatcatcaacctgtggttgggtgcacacctggttatttccggggatttaagtattggtcagttaattgcttttaatatgcttgcaggtcagattgttgcaccggttattcgccttgcacaaatctggcaggatttccagcaggttggtatatcagttacccgccttggtgatgtgcttaactctccaactgaaagttatcatgggaaactggcattaccggaaattaatggtgatatcacttttcgtaatatccggtttcgctataagcctgactctccggttattttagataatatcaatctcagtattaagcagggggaggttattggtattgtcggacgttctggttcaggaaaaagcacattaactaaattaattcaacgtttttatattcctgaaaatggccaggtcttaattgatggacatgatcttgcgttggccgatcctaactggttacgtcgtcaggtgggggttgtgttgcaggacaatgtgctgcttaatcgcagtattattgataatatttcactggctaatcctggcatgtccgtcgaaaaagttatttatgcagcgaaattagcaggtgctcatgattttatttctgaattgcgtgaggggtataacaccattgtcggggaacagggggcaggattatccggaggtcaacgtcaacgcatcgcaattgcaagggcgctggtgaacaaccctaaaatactcatctttgatgaagcaaccagtgctctggattatgagtcggagcatgtcatcatgcgcaatatgcacaaaatatgtaagggcagaacggttataatcattgctcatcgtctgtctacagtaaaaaatgcagaccgcattattgtcatggaaaaagggaaaattgttgaacagggtaaacataaggagctgctttctgaaccggaaagtttatacagttacttatatcagttacagtcagactaacagaaagaacagaagaatatgaaaacatggttaatggggttcagcgagttcctgttgcgctataaacttgtctggagtgaaacatggaaaatccggaagcagttagatactccggtacgtgaaaaggacgaaaatgaattcttacccgctcatctggaattaattgaaacgccggtatccagacggccgcgtctggttgcttattttattatggggtttctggttattgctttcattttatctgttttaggccaggtggaaattgttgccactgcaaatgggaaattaacactcagtgggcgtagcaaagaaattaaacctattgaaaactcgatagttaaagaaattatcgtaaaagaaggagagtcagtccggaaaggggatgtgttattaaagcttacagcgctgggagctgaagctgatacgttaaaaacgcagtcatcactgttacaggccaggctggaacaaattcggtatcaaattctgagccggtcaattgaattaaataaacttcctgaactgaaacttcctgatgagccttattttcagaatgtatctgaagaggaagtactgcgtttaacttctttgataaaagaacagttttccacatggcaaaatcagaagtatcaaaaagaactgaatctggataagaaaagagcagagcgattaacaatacttgcccgtataaaccgttatgaaaatgtatcgagggttgaaaaaagccgtctggatgatttcaggagcctgttgcataaacaggcaattgcaaaacatgctgtacttgagcaggagaataaatatgttgaggcagcaaatgaattacgggtttataaatcgcaactggagcaaattgagagtgagatattgtctgcaaaagaagaatatcagcttgtcacgcagctttttaaaaatgaaattttagacaagctaagacaaacaacagacagcattgagttattaactctggagttagagaaaaatgaagagcgtcaacaggcttcagtaatcagggcccctgtttcgggaaaagttcagcaactgaaggttcatactgaaggtggggttgttacaacagcggaaacactgatggtcatcgttccggaagatgacacgctggaggttactgctctggtacaaaataaagatattggttttattaacgtcgggcagaatgccatcattaaagtggaggcttttccttacacccgatatggttatctggtgggtaaggtaaaaaatataaatttagatgcaatagaagaccagaaactgggactcgtttttaatgtcattgtttctgttgaagagaatgatttgtcaaccgggaataagcacattccattaagctcgggtatggctgtcactgcagaaataaagactggaatgcgaagcgtaatcagttatcttcttagtcccctggaagaatctgtaacagaaagtttacatgagcgttaagtctcagagcagcggtatccggctcatatcttctcctgtcagatccaagggcgaattccagcacactggcggccgttactagtggatccaccggtctcgaggggcgcgccggtaccgaattctaattaatgggcatcatttttcagctatttcatttataaaataagttatgaaaaaaatcatcatattactaacgacatttttcctgctttcgggatgcactattcccagggcggtatttaaatccagccttattaatcaggacgatcctcgttataatctggtcgaagtcacgccgacattaaaactaagcgctcccgatactgtgccgaaaactattgtcgatccggtttttgccgcaaataactggcactggacatctttggctaaaggcgatgtgctgcatatcactattttatcctcgggcggggctggatatttatccaataacgcgagcggcgaccgtgcggattttgaaaatattcttgtgactgacagtaataccgttcaggtgccttatgccgggacaatcccggtttctggattggatgtgacgcaactggctgatgagatcaaaaagcgactttcgcgcgttgtcctgaatcctcaggtgattgtgacacttaccgcccgcaccggagccatggtgacggtcgagggcagcgggaaaacgggtcgataccctctcgaacagagtatgaatcgtctgagtcatcttttggcaacggcagtggcagtagaaaataccagcacagatatgatggaagttcacgtgacgcgccagcagcattatttcactgcgcgactgtcagatatttatcagtatcccggattggatattgccttacagccggatgaccgtatcactctgcgtcaagtgaccgagtatgttaatgtgctcggcgcagcaggtgttcaggggaaacatgctctggttcagcgtcattccagcgtcgtggatgctttggcgctcgcgaaaggattaaatgacaatcttgccgatccgcaagcgatttttctgtacaagcataacgaagcggaacaggcgaaacagcagatgcgtaagctgaatatctatcatgtcgatatgagccaacctaactcggtgtttttagcgcaggccatacgagtggacaacggcgatgttatctatatctcgaacgcctctttgactgatttcgctaaggtgaaagccgcattcgatagctttttgac

SEQ ID NO: 11 - protein, 223 aa, Artificial Sequence
  SARS-CoV-2 RBD
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 12 - protein, 27 aa, Artificial Sequence
  (T4 Fibritin Trimerization Motif
GYIPEAPRDGQAYVRKDGEWVLLSTFL

SEQ ID NO: 13 - protein, 61 aa, Artificial Sequence
  HLYA signal sequence
ALAYGSQGNLNPLINEISKIISAAGNFDVKEERAAASLLQLSGNASDFSYGRNSITLTASA

SEQ ID NO: 14 - protein, 310 aa, Artificial Sequence
  YbaS
MLDANKLQQAVDQAYTQFHSLNGGQNADYIPFLANVPGQLAAVAIVTCDGNVYSAGDSDYRFALESISKVCTLALALEDVGPQAVQDKIGADPTGLPFNSVIALELHGGKPLSPLVNAGAIATTSLINAENVEQRWQRILHIQQQLAGEQVALSDEVNQSEQTTNFHNRAIAWLLYSAGYLYCDAMEACDVYTRQCSTLINTVELATLGATLAAGGVNPLTHKRVLQADNVPYILAEMMMEGLYGRSGDWAYRVGLPGKSGVGGGILAVVPGVMGIAAFSPPLDEEGNSVRGQKMVASVAKQLGYNVFKG

SEQ ID NO: 15 - DNA, 933 bp, Artificial Sequence
  YbaS
  misc_feature (141)..(141): n is a, c, g, or t
atgttagatgcaaacaaattacagcaggcagtggatcaggcttacacccaatttcactcacttaacggcggacaaaatgccgattacattccctttctggcgaatgtaccaggtcaactggcggcagtggctatcgtgacntgcgatggcaacgtctatagtgcgggtgacagtgattaccgctttgcactggaatccatctctaaagtctgtacgttagcccttgcgttagaagatgtcggcccgcaggcggtacaggacaaaattggcgctgacccgaccggattgccctttaactcagttatcgccttagagttgcatggcggcaaaccgctgtcgccactggtaaatgctggcgctattgccaccaccagcctgattaacgctgaaaatgttgaacaacgctggcagcgaattttacatatccaacagcaactggctggtgagcaggtagcgctctctgacgaagtcaaccagtcggaacaaacaaccaacttccataaccgggccattgcctggctgctgtactccgccggatatctctattgtgatgcaatggaagcctgtgacgtgtacacccgtcagtgctctacgctcatcaatactgttgaactggcaacgcttggcgcgacgctggcggcgggtggtgtgaatccgttgacgcataaacgcgttcttcaggccgacaacgtgccgtacattctggccgaaatgatgatggaagggctgtatggtcgctccggtgactgggcgtatcgcgttggtttaccgggcaaaagcggtgtaggtggcggtattctggcggtcgtccctggcgtgatgggaattgccgcgttctcaccaccgctggacgaagaaggcaacagtgttcgcggtcaaaaaatggtggcatcggtcgctaagcaactcggctataacgtgtttaagggctga

SEQ ID NO: 16 - protein, 476 aa, Artificial Sequence
  GadB
MRSNHFKEFKMDKKQVTDLRSELLDSRFGAKSISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 17 - DNA, 1431 bp, Artificial Sequence
  GadB
atgcgatccaatcattttaaggagtttaaaatggataagaagcaagtaacggatttaaggtcggaactactcgattcacgttttggtgcgaagtctatttccactatcgcagaatcaaaacgttttccgctgcacgaaatgcgcgacgatgtcgcatttcagattatcaatgatgaattatatcttgatggcaacgctcgtcagaacctggccactttctgccagacctgggacgacgaaaacgtccacaagttgatggatttatccattaacaaaaactggatcgacaaagaagaatatccgcaatccgcagccatcgacctgcgttgcgtaaacatggttgccgatctgtggcatgcgcctgcgccgaaaaatggtcaggccgttggcaccaacaccattggttcttccgaggcctgtatgctcggcgggatggcgatgaaatggcgttggcgcaagcgtatggaagctgcaggcaaaccaacggataaaccaaacctggtgtgcggtccggtgcaaatctgctggcataaattcgcccgctactgggatgtggagctgcgtgagatccctatgcgccccggtcagttgtttatggacccgaaacgcatgattgaagcctgcgacgaaaataccatcggcgtggtgccgactttcggcgtgacctacaccggtaactatgagttcccgcaaccgctgcacgatgcactggataaattccaggccgacaccggtatcgacatcgacatgcacatcgacgccgccagcggtggctttctggcaccgttcgtcgccccggatatcgtctgggacttccgcctgccgcgtgtgaaatcgatcagtgcttcaggccataaattcggtctggctccgctgggctgcggctgggttatctggcgtgacgaagaagcgctgccgcaggaactggtgttcaacgttgactacctcggtggtcagattggtacttttgccatcaacttctcccgcccggcgggtcaggtgattgcacagtactatgaattcctgcgccttggtcgtgaaggctataccaaagtacagaatgcttcctaccaggtcgctgcctatctggcggatgagatcgccaaactggggccgtatgagttcatctgtaccggtcgcccggacgaaggcatcccggcggtttgcttcaaactgaaagatggtgaagatccgggatacaccctgtacgacctctctgaacgtctgcgtctgcgcggctggcaggttccggccttcactctcggcggtgaagccaccgacatcgtggtgatgcgcattatgtgtcgtcgcggcttcgaaatggactttgctgaactgttgctggaagactacaaagcctccctgaaatatctcagcgatcacccgaaactgcagggtattgcccagcagaacagctttaaacatacctga

SEQ ID NO: 18 - protein, 511 aa, Artificial Sequence
  GadC
MATSVQTGKAKQLTLLGFFAITASMVMAVYEYPTFATSGFSLVFFLLLGGILWFIPVGLCAAEMATVDGWEEGGVFAWVSNTLGPRWGFAAISFGYLQIAIGFIPMLYFVLGALSYILKWPALNEDPITKTIAALIILWALALTQFGGTKYTARIAKVGFFAGILLPAFILIALAAIYLHSGAPVAIEMDSKTFFPDFSKVGTLVVFVAFILSYMGVEASATHVNEMSNPGRDYPLAMLLLMVAAICLSSVGGLSIAMVIPGNEINLSAGVMQTFTVLMSHVAPEIEWTVRVISALLLLGVLAEIASWIVGPSRGMYVTAQKNLLPAAFAKMNKNGVPVTLVISQLVITSIALIILTNTGGGNNMSFLIALALTVVIYLCAYFMLFIGYIVLVLKHPDLKRTFNIPGGKGVKLVVAIVGLLTSIMAFIVSFLPPDNIQGDSTDMYVELLVVSFLVVLALPFILYAVHDRKGKANTGVTLEPINSQNAPKGHFFLHPRARSPHYIVMNDKKH

SEQ ID NO: 19 - DNA, 1536 bp, Artificial Sequence
  GadC
atggctacatcagtacagacaggtaaagctaagcagctcacattactcggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctgggtccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttacctgcactccggtgcccccgttgctatcgaaatggattcgaagaccttcttccctgacttctctaaagtgggcacactggttgtatttgttgccttcattttgagttatatgggcgtagaagcttccgcaactcacgtcaatgaaatgagcaaccccgggcgcgactatccactggctatgttactgctgatggtggcggcaatctgcttaagctctgttggcggtttgtctattgcgatggtcattccgggtaatgaaatcaacctctccgcaggggtaatgcaaacctttaccgttctgatgtcccatgtggcaccggaaattgagtggacggttcgcgtgatctccgcactgctgttgctgggtgttctggcggaaatcgcctcctggattgttggtccttctcgcgggatgtatgtcacagcgcagaaaaacctgctgccagcggcattcgctaaaatgaacaaaaatggcgtaccggtaacgctggtcatttcgcagctggtgattacttctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaaggcaaagcgaataccggcgtcactctggagccaatcaacagtcagaacgcaccaaaaggtcacttcttcctgcacccgcgtgcacgttcaccacactatattgtgatgaatgacaagaaacactaa

SEQ ID NO: 20 - DNA, 5178 bp, Artificial Sequence
  20 (araC-YbaS-GadBC AR cassette)
  misc_feature (1377)..(1377): n is a, c, g, or t
cgatcgttatgacaacttgacggctacatcattcactttttcttcacaaccggcacggaactcgctcgggctggccccggtgcattttttaaatacccgcgagaagtagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaataacacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctcggagttaacaaaagatgttagatgcaaacaaattacagcaggcagtggatcaggcttacacccaatttcactcacttaacggcggacaaaatgccgattacattccctttctggcgaatgtaccaggtcaactggcggcagtggctatcgtgacntgcgatggcaacgtctatagtgcgggtgacagtgattaccgctttgcactggaatccatctctaaagtctgtacgttagcccttgcgttagaagatgtcggcccgcaggcggtacaggacaaaattggcgctgacccgaccggattgccctttaactcagttatcgccttagagttgcatggcggcaaaccgctgtcgccactggtaaatgctggcgctattgccaccaccagcctgattaacgctgaaaatgttgaacaacgctggcagcgaattttacatatccaacagcaactggctggtgagcaggtagcgctctctgacgaagtcaaccagtcggaacaaacaaccaacttccataaccgggccattgcctggctgctgtactccgccggatatctctattgtgatgcaatggaagcctgtgacgtgtacacccgtcagtgctctacgctcatcaatactgttgaactggcaacgcttggcgcgacgctggcggcgggtggtgtgaatccgttgacgcataaacgcgttcttcaggccgacaacgtgccgtacattctggccgaaatgatgatggaagggctgtatggtcgctccggtgactgggcgtatcgcgttggtttaccgggcaaaagcggtgtaggtggcggtattctggcggtcgtccctggcgtgatgggaattgccgcgttctcaccaccgctggacgaagaaggcaacagtgttcgcggtcaaaaaatggtggcatcggtcgctaagcaactcggctataacgtgtttaagggctgaccatggagtgttttaatgcgatccaatcattttaaggagtttaaaatggataagaagcaagtaacggatttaaggtcggaactactcgattcacgttttggtgcgaagtctatttccactatcgcagaatcaaaacgttttccgctgcacgaaatgcgcgacgatgtcgcatttcagattatcaatgatgaattatatcttgatggcaacgctcgtcagaacctggccactttctgccagacctgggacgacgaaaacgtccacaagttgatggatttatccattaacaaaaactggatcgacaaagaagaatatccgcaatccgcagccatcgacctgcgttgcgtaaacatggttgccgatctgtggcatgcgcctgcgccgaaaaatggtcaggccgttggcaccaacaccattggttcttccgaggcctgtatgctcggcgggatggcgatgaaatggcgttggcgcaagcgtatggaagctgcaggcaaaccaacggataaaccaaacctggtgtgcggtccggtgcaaatctgctggcataaattcgcccgctactgggatgtggagctgcgtgagatccctatgcgccccggtcagttgtttatggacccgaaacgcatgattgaagcctgcgacgaaaataccatcggcgtggtgccgactttcggcgtgacctacaccggtaactatgagttcccgcaaccgctgcacgatgcactggataaattccaggccgacaccggtatcgacatcgacatgcacatcgacgccgccagcggtggctttctggcaccgttcgtcgccccggatatcgtctgggacttccgcctgccgcgtgtgaaatcgatcagtgcttcaggccataaattcggtctggctccgctgggctgcggctgggttatctggcgtgacgaagaagcgctgccgcaggaactggtgttcaacgttgactacctcggtggtcagattggtacttttgccatcaacttctcccgcccggcgggtcaggtgattgcacagtactatgaattcctgcgccttggtcgtgaaggctataccaaagtacagaatgcttcctaccaggtcgctgcctatctggcggatgagatcgccaaactggggccgtatgagttcatctgtaccggtcgcccggacgaaggcatcccggcggtttgcttcaaactgaaagatggtgaagatccgggatacaccctgtacgacctctctgaacgtctgcgtctgcgcggctggcaggttccggccttcactctcggcggtgaagccaccgacatcgtggtgatgcgcattatgtgtcgtcgcggcttcgaaatggactttgctgaactgttgctggaagactacaaagcctccctgaaatatctcagcgatcacccgaaactgcagggtattgcccagcagaacagctttaaacatacctgataaccgttttggagtgataatatggctacatcagtacagacaggtaaagctaagcagctcacattactcggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctgggtccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttacctgcactccggtgcccccgttgctatcgaaatggattcgaagaccttcttccctgacttctctaaagtgggcacactggttgtatttgttgccttcattttgagttatatgggcgtagaagcttccgcaactcacgtcaatgaaatgagcaaccccgggcgcgactatccactggctatgttactgctgatggtggcggcaatctgcttaagctctgttggcggtttgtctattgcgatggtcattccgggtaatgaaatcaacctctccgcaggggtaatgcaaacctttaccgttctgatgtcccatgtggcaccggaaattgagtggacggttcgcgtgatctccgcactgctgttgctgggtgttctggcggaaatcgcctcctggattgttggtccttctcgcgggatgtatgtcacagcgcagaaaaacctgctgccagcggcattcgctaaaatgaacaaaaatggcgtaccggtaacgctggtcatttcgcagctggtgattacttctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaaggcaaagcgaataccggcgtcactctggagccaatcaacagtcagaacgcaccaaaaggtcacttcttcctgcacccgcgtgcacgttcaccacactatattgtgatgaatgacaagaaacactaacgatcg

SEQ ID NO: 21 - protein, 466 aa, Artificial Sequence
  GadA
MDQKLLTDFRSELLDSRFGAKAISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKQGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 22 - DNA, 1401 bp, Artificial Sequence
  GadA
atggaccagaagctgttaacggatttccgctcagaactactcgattcacgttttggcgcaaaggccatttctactatcgcggagtcaaaacgatttccgctgcacgaaatgcgcgatgatgtcgcattccagattatcaatgatgaattatatcttgatggcaacgctcgtcagaacctagccactttctgccagacctgggacgacgaaaacgtccataaattgatggatttgtcgatcaataaaaactggatcgacaaagaagaatatccgcaatccgcagccatcgacctgcgttgcgtaaatatggttgccgatctgtggcatgcgcctgcgccgaaaaatggtcaggccgttggcaccaacaccattggttcttccgaggcctgtatgctcggcgggatggcgatgaaatggcgttggcgcaagcgtatggaagctgcaggcaaaccaacggataaaccaaacctagtgtgcggtccggtacaaatctgctggcataaattcgcccgctactgggatgtggagctgcgtgagatccctatgcgccccggtcagttgtttatggacccgaaacgcatgattgaagcctgtgacgaaaacaccatcggcgtggtgccgactttcggcgtgacctacaccggtaactatgagttcccacaaccgctgcacgatgcgctggataaattccaggccgacaccggtatcgacatcgacatgcacatcgacgctgccagcggtggcttcctggcaccgttcgtcgccccggatatcgtctgggacttccgcctgccgcgtgtgaaatcgatcagtgcttcaggccataaattcggtctggctccgctgggctgcggctgggttatctggcgtgacgaagaagcgctgccgcaggaactggtgttcaacgttgactacctgggtggtcaaattggtacttttgccatcaacttctcccgcccggcgggtcaggtaattgcacagtactatgaattcctgcgcctcggtcgtgaaggctataccaaagtacagaacgcctcttaccaggttgccgcttatctggcggatgaaatcgccaaacaggggccgtatgagttcatctgtacgggtcgcccggacgaaggcatcccggcggtttgcttcaaactgaaagatggtgaagatccgggatacaccctgtacgacctctctgaacgtctgcgtctgcgcggctggcaggttccggccttcactctcggcggtgaagccaccgacatcgtggtgatgcgcattatgtgtcgtcgcggcttcgaaatggactttgctgaactgttgctggaagactacaaagcctccctgaaatatctcagcgatcacccgaaactgcagggtattgcccagcagaacagctttaaacacacctga

(19) United States
(12) Patent Application Publication
(10) Pub. No.: US 2022/0233681 A1
(43) Pub. Date: Jul. 28, 2022

(54) LIVE ATTENUATED ORAL VACCINE AGAINST COVID-19 AND TYPHOID FEVER

(71) Applicant: Protein Potential, LLC, Rockville, MD (US)

(72) Inventors: Betty Kim Lee SIM, Gaithersburg, MD (US); Tint Tint WAI, Germantown, MD (US); Ehud INBAR, Rockville, MD (US); Stephen L. HOFFMAN, Gaithersburg, MD (US)

(21) Appl. No.: 17/566,901

(22) Filed: Dec. 31, 2021

Related U.S. Application Data
(60) Provisional application No. 63/133,131, filed on Dec. 31, 2020.

(51) Int. Cl. A61K 39/215; A61P 31/14

(52) U.S. Cl. CPC A61K 39/215; A61K 2039/523; A61P 31/14

(57) ABSTRACT

Disclosed herein are transgenic Salmonella typhi Ty21a comprising a chromosome with one or more heterologous nucleic acid regions, wherein the heterologous nucleic acid regions encode one or more viral antigens and are integrated into the Salmonella typhi Ty21a chromosome, and wherein the transgenic Salmonella typhi Ty21a stably expresses the one or more viral antigens. Also disclosed herein are compositions and vaccines comprising the transgenic Salmonella typhi Ty21a. Also disclosed herein are methods of eliciting an immune response in a subject against a SARS-CoV-2 viral antigen and/or a Salmonella typhi antigen comprising administering one or more doses of the composition or the vaccine to the subject. Also disclosed herein are methods of treating, preventing or reducing the incidence of COVID-19 and/or typhoid fever in a subject comprising administering one or more doses of the composition or the vaccine to the subject.

### CROSS REFERENCE TO RELATED APPLICATIONS

The present application claims priority benefit of U.S. Provisional Application No. 63/133,131, filed Dec. 31, 2020, which is hereby incorporated by reference in its entirety.

### REFERENCE TO SEQUENCE LISTING SUBMITTED ELECTRONICALLY VIA EFS-WEB

The content of the electronically submitted sequence listing in ASCII text file format (Name: 4661_0010001_Seqlisting_ST25.txt; Size: 158,919 Bytes; and Date of Creation: Dec. 22, 2021) is hereby incorporated by reference in its entirety.

### FIELD OF THE DISCLOSURE

The present disclosure is generally related to bioengineering and provides multivalent vaccines for protection against COVID-19 and/or typhoid fever.

### BACKGROUND OF THE DISCLOSURE

Severe acute respiratory syndrome-coronavirus 2 (SARS-CoV-2), the causative agent of COVID-19, is spreading globally in the most significant pandemic of the past 100 years (World Health Organization, WHO Director-General's opening remarks at the media briefing on COVID-19-11 (March 2020); World Health Organaization, Timeline of WHO's response to COVID-19. 29 Jun. 2020, https://www.who.int/news-room/detail/29-06-2020-covidtimeline). As of 27 Dec. 2020, there were 79,232,555 million cases and 1,754,493 deaths worldwide (GISAID, Coronavirus COVID-19 Global Cases by Johns Hopkins CSSE, https://www.gisaid.org/epiflu-applications/global-cases-covid-19/ (2020)). As of the same time, an approved vaccine was urgently needed. As of December 2020, there were about 236 COVID-19 vaccine approaches under development (FastCures, The Milken Institute, COVID-19 treatment and vaccine tracker (2020), https://covid-19tracker.milkeninstitute.org/). The most advanced approach (using mRNA technologies) resulted in 2 successful candidates being rolled out to the public (https://www.fda.gov/emergency-preparedness-and-response/coronavirus-disease-2019-covid-19/pfizer-biontech-covid-19-vaccine; https://www.fda.gov/emergency-preparedness-and-response/coronavirus-disease-2019-covid-19/moderna-covid-19-vaccine).

Other candidates include recombinant chimpanzee adenovirus, nucleic acid (mRNA and DNA), and protein/adjuvant vaccines, all expressing or containing the spike (S) glycoprotein of SARS-CoV-2. Many approaches suffer from the downstream challenges to scale up manufacturing of these vaccines (and adjuvant) and implement widespread vaccination in a safe and efficient manner. However, all these candidates likely will require storage and shipment at <−60° C., and widespread distribution at these temperatures will require dry ice for shipping and ultra-low freezers for storage, which in turn are dependent on a reliable electrical source. These requirements are problematic for many of the health care facilities that will be administering the vaccine, including physician's offices and pharmacies all over the world, as such facilities rarely have ultra-low freezers. In addition, in low and middle-income countries, electricity is minimal, unreliable and/or absent in many areas and populations, creating a significant challenge for vaccine distribution. Finally, given that COVID-19 is highly infectious and easily spread, requiring populations to congregate at hospitals, clinics or vaccine administration centers to receive vaccination (likely requiring multiple visits) will pose a confounding problem.

Salmonella Typhi Ty21a typhoid vaccine (Vivotif®) (Kopecko, D. J., et al., Int J Med Microbiol, 299(4):233-46 (2009)) is one of only a few live, oral, attenuated bacterial vaccines licensed in the US. Ty21a, when administered for a one week period, affords sustained protection from typhoid fever for 7 years with efficacies ranging from 62-96% as reported in Chilean/Egyptian field trials (Levine, M. M. Typhoid fever vaccines. In: Plotkin, S. A., Orenstein, W. A., eds. Vaccines, 3^{rd} ed. Philadelphia, Pa., WB Saunders Company, 1999:781-814; Levine, M. M., et al., Vaccine 17 Suppl 2:S22-7 (1999); Wandan, M. H., et al., J Infect Dis. 145(3):292-5 (1982)), and it has had an unrivaled safety record during the past 25 years (Gilman, R. H., et al., J Infect Dis. 136(6):717-23 (1977); Cryz, S. J., Jr., Lancet 341(8836):49-50 (1993); Simanjuntak, C. H., et al., Lancet 338(8774):1055-9 (1991); Levine, M. M., et al., The efficacy of attenuated Salmonella typhi oral vaccine strain Ty21a evaluated in controlled field trials. In: J. Holmgren, A. Lindberg, and R. Mollby, eds. Development of vaccines against diarrhea. 11th Nobel Conference, Stockholm, 1985. Studentliteratur, Lund, Sweden, 1986:90-101). As of 2013, there has never been a reported case of bacteremic dissemination of Ty21a after administration to more than 150 million recipients (Kopecko, D. J., et al., Int J Med Microbiol. 299(4):233-46 (2009)), and Ty21a is nonpathogenic even when given at 100 times the standard dose (Levine, M. M., et al., Vaccine 17 Suppl 2:S22-7 (1999)). Also, there are no reports of post-vaccination inflammatory arthritis (e.g., Reiter's syndrome) with Ty21a, a potential problem with other live attenuated vectors including non-typhoid Salmonella, Shigella, and Yersinia. Ty21a can be foam-dried, which provides for temperature stabilization (Ohtake, S., et al., Vaccine 29(15): p. 2761-71 (2011)).

Ty21a has been used for expression of heterologous antigens from plasmids (U.S. Pat. Nos. 7,541,043, 8,071,084, 8,337,832, and 8,992,943; Chin'ombe, N., Viruses 5(9):2062-78 (2013); and Frey, S. E., et al., Vaccine 31(42):4874-80 (2013)). Additionally, recombinant genome-integrated Ty21a strains have been used to stably express the 0-antigens of Shigella sonnei (U.S. Pat. Nos. 9,750,793 and 10,695,415), and Ty21a has been used to express the major virulence factors of enterotoxigenic Escherichia coli (ETEC) (Wai, T. et al., Amer Soc Trop Med & Hygiene 101:539 (2019); U.S. Publication No. 2020/0376107 A1) and the protective antigen (PA) of B. anthracis (Sim, B. K. L., et al., NPJ Vaccines 2:17 (2017). A recent study described the beneficial nonspecific effects of oral vaccination with live-attenuated Salmonella Typhi strain Ty21a through the innate immune system (Pennington, S. H., et al., Sci Adv. 5(2):eaau6849 (2019)).

To date, there is no reported use of live attenuated Ty21a for expression of a heterologous viral antigen, nor use of such a vector to treat, prevent, or reduce the incidence of a viral infection.

### SUMMARY OF THE DISCLOSURE

The present disclosure relates generally to the development of bivalent or multivalent, live, attenuated compositions (e.g., vaccine compositions) for protection against COVID-19 caused by SARS-CoV-2. Disclosed herein are constructed vaccine strains of Salmonella typhi in which one or more SARS-CoV-2-specific antigenic sequences from the spike (S), membrane (M), and/or nucleocapsid (N) proteins are recombineered into the chromosome of Salmonella typhi Ty21a such that the one or more antigenic sequences are stably expressed. The resulting recombineered Ty21a-SARS-CoV-2 strains can be used, for example, in orally or intranasally administered vaccine preparations to elicit an immune response to said antigens and to treat, prevent, or reduce the incidence of COVID-19 in an individual. In some aspects, the recombineered Ty21a-SARS-CoV-2 strains of the present disclosure are constructed to co-express the concerted YbaS-GadBC acid resistance (AR) system (SEQ ID NO: 20) and the one or more SARS-CoV-2-specific antigenic sequences.

Certain aspects of the present disclosure are directed to a transgenic Salmonella typhi Ty21a comprising a chromosome with one or more heterologous nucleic acid regions, wherein the heterologous nucleic acid regions encode one or more SARS-CoV-2 viral antigens and are integrated into the Salmonella typhi Ty21a chromosome, and wherein the transgenic Salmonella typhi Ty21a stably expresses the one or more SARS-CoV-2 viral antigens.

In some aspects, the heterologous nucleic acid regions comprise: (a) a nucleic acid sequence encoding a SARS-CoV-2 spike (S) protein or one or more antigenic fragments thereof, (b) a nucleic acid sequence encoding one or more antigens retaining at least 70%, at least 75%, at least 80%, at least 85%, at least 90%, at least 95%, at least 96%, at least 97%, at least 98%, at least 99%, or 100% amino acid residue homology to a native SARS-CoV-2 S protein antigen, (c) a nucleic acid sequence encoding a SARS-CoV-2 S protein ectodomain (eS) or one or more antigenic fragments thereof, (d) a nucleic acid encoding one or more antigens retaining at least 70%, at least 75%, at least 80%, at least 85%, at least 90%, at least 95%, at least 96%, at least 97%, at least 98%, at least 99%, or 100% amino acid residue homology to a native SARS-CoV-2 eS antigen, (e) a nucleic acid sequence encoding a SARS-CoV2 Membrane (M) protein or one or more antigenic fragments thereof, (f) a nucleic acid sequence encoding one or more antigens retaining at least 70%, at least 75%, at least 80%, at least 85%, at least 90%, at least 95%, at least 96%, at least 97%, at least 98%, at least 99%, or 100% amino acid residue homology to a native SARS-CoV-2 M protein antigen, (g) a nucleic acid sequence encoding a SARS-CoV2 Nucleocapsid (N) protein or one or more antigenic fragments thereof, (h) a nucleic acid sequence encoding one or more antigens retaining at least 70%, at least 75%, at least 80%, at least 85%, at least 90%, at least 95%, at least 96%, at least 97%, at least 98%, at least 99%, or 100% amino acid residue homology to a native SARS-CoV-2 N protein antigen, or (i) any combination thereof.

In some aspects, the heterologous nucleic acid regions encode a full length SARS-CoV-2 M protein. In some aspects, the full length SARS-CoV-2 M protein comprises the amino acid sequence of SEQ ID NO: 2.

In some aspects, the heterologous nucleic acid regions encode a full length SARS-CoV-2 N protein. In some aspects, the full length SARS-CoV-2 M protein comprises the amino acid sequence of SEQ ID NO: 3.

In some aspects, the heterologous nucleic acid regions encode a modified SARS-CoV-2 S protein Receptor Binding Domain (RBD) or a modified SARS-CoV-2 eS. In some aspects, the modified SARS-CoV-2 S protein RBD or the modified SARS-CoV-2 eS comprises a D614G substitution. In some aspects, the modified SARS-CoV-2 S protein RBD or the modified SARS-CoV-2 eS comprises a GSAS substitution at amino acid residues 682-685. In some aspects, the modified SARS-CoV-2 S protein RBD or the modified SARS-CoV-2 eS comprises proline substitutions at amino acid residues 986 and 987. In some aspects, the modified SARS-CoV-2 S protein RBD or the modified SARS-CoV-2 eS comprises a C-terminal T4 fibritin trimerization motif (SEQ ID NO: 12). In some aspects, the modified SARS-CoV-2 S protein RBD or the modified SARS-CoV-2 eS comprises F817P, A892P, A899P, and A942P substitutions. In some aspects, the modified SARS-CoV-2 RBD comprises the amino acid sequence of SEQ ID NO: 11. In some aspects, the modified SARS-CoV-2-eS comprises a hemolysin A (HLYA) signal sequence (SEQ ID NO: 13). In some aspects, the modified SARS-CoV-2 eS comprises the amino acid sequence of SEQ ID NO: 1 or SEQ ID NO: 5. In some aspects, the heterologous nucleic acid regions encoding the modified SARS-CoV-2 eS comprise a nucleic acid sequence at least 70%, at least 75%, at least 80%, at least 85%, at least 90% at least 95%, at least 99%, or 100% identical to the nucleic acid sequence of SEQ ID NO: 6.

In some aspects, the heterologous nucleic acid regions encoding the modified SARS-CoV-2 S protein RBD or the modified SARS-CoV-2 eS further encode a full length SARS-CoV2 M protein and a full length SARS-CoV2 N protein. In some aspects, the full length SARS-CoV-2 M protein comprises the amino acid sequence of SEQ ID NO: 2, and the full length SARS-CoV-2 N protein comprises the amino acid sequence of SEQ ID NO: 3.

In some aspects, the heterologous nucleic acid regions encode a full length SARS-CoV-2 M protein and a full length SARS-CoV-2 N protein. In some aspects, the full length SARS-CoV-2 M protein comprises the amino acid sequence of SEQ ID NO: 2, and the full length SARS-CoV-2 N protein comprises the amino acid sequence of SEQ ID NO: 3. In some aspects, the heterologous nucleic acid regions encoding the full length SARS-CoV-2 M protein and the full length SARS-CoV-2 N protein comprise a nucleic acid sequence at least 70%, at least 75%, at least 80%, at least 85%, at least 90% at least 95%, at least 99%, or 100% identical to the nucleic acid sequence of SEQ ID NO: 7.

In some aspects, the transgenic Salmonella typhi Ty21a further comprises a heterologous acid resistance gene cassette integrated into the Salmonella typhi Ty21a chromosome. In some aspects, the heterologous acid resistance gene cassette comprises a gene selected from the group consisting of: a YbaS gene (SEQ ID NO: 15) or a fragment thereof, a GadB gene (SEQ ID NO: 17) or fragment thereof, a GadC gene (SEQ ID NO: 19) or fragment thereof, a GadA gene or fragment thereof (SEQ ID NO: 22), and any combination thereof. In some aspects, the heterologous acid resistance gene cassette comprises a sequence having at least 80%, 85%, 90%, 95%, or 100% sequence identity to one or more nucleic acid sequences selected from: SEQ ID NO: 15, SEQ ID NO: 17, SEQ ID NO: 19, SEQ ID NO: 20, and SEQ ID NO: 22. In some aspects, the heterologous nucleic acid regions of the transgenic Salmonella typhi Ty21a comprising the heterologous acid resistance gene cassette comprise a nucleic acid sequence at least 70%, at least 75%, at least 80%, at least 85%, at least 90% at least 95%, at least 99%, or 100% identical to the nucleic acid sequence of SEQ ID NO: 8, SEQ ID NO: 9, or SEQ ID NO: 10. In some aspects, the transgenic Salmonella typhi Ty21a comprising the heterologous acid resistance gene cassette is more acid stable at pH 2.5 than Salmonella typhi Ty21a without the integrated acid resistance gene.

Certain aspects of the present disclosure are directed to a composition, e.g., a bacterial composition, comprising a transgenic Salmonella typhi Ty21a disclosed herein in combination with a carrier, e.g., such that the carrier renders the composition suitable for pharmaceutical use.

Certain aspects of the present disclosure are directed to a vaccine comprising a composition disclosed herein. Certain aspects of the present disclosure are directed to a vaccine comprising a transgenic Salmonella typhi Ty21a disclosed herein in combination with a carrier, e.g., such that the carrier renders the vaccine suitable for pharmaceutical use. In some aspects, the vaccine is suitable for oral or inhaled administration.

In some aspects, the vaccine is protective against COVID-19. In some aspects, the vaccine is protective against typhoid fever. In some aspects, the vaccine is protective against COVID-19 and typhoid fever.

In some aspects, the vaccine is foam dried. In some aspects, the vaccine is foam dried, rendering the vaccine at least 90% stable, at least 95% stable, at least 99% stable, or 100% stable at room temperature for at least one month. In some aspects, the vaccine is foam dried, rendering the vaccine at least 40% stable, at least 50% stable, at least 60% stable, at least 70% stable, at least 80% stable, at least 90% stable, at least 95% stable, at least 99% stable, or 100% stable at room temperature for at least one year. In some aspects, the vaccine is foam dried, rendering the vaccine at least 40% stable, at least 50% stable, at least 60% stable, at least 70% stable, at least 80% stable, at least 90% stable, at least 95% stable, at least 99% stable, or 100% stable at room temperature for at least two years. In some aspects, the vaccine is foam dried and has a shelf-life of at least 3 months, at least 6 months, at least 9 months, at least 1 year, at least 2 years, at least 3 years, at least 4 years, at least 5 years at 4° C. In some aspects, the vaccine is foam dried and has a shelf-life of 6-24 months, 1-2 years, 1-5 years, 1-10 years, or 5-10 years at 4° C.

In some aspects, administration of a vaccine or composition of the present disclosure to one or more individuals or a human population reduces the incidence of COVID-19 in those individuals or that human population subsequently exposed to pathogenic SARS-CoV-2.

In some aspects, administration of a vaccine or composition of the present disclosure to one or more individuals or a human population reduces the incidence of typhoid fever in those individuals or that human population subsequently exposed to pathogenic S. typhi.

In some aspects, administration of a vaccine or composition of the present disclosure to one or more individuals or a human population reduces the incidence of both COVID-19 and typhoid fever in those individuals or that human population subsequently exposed to both pathogenic SARS-CoV-2 and pathogenic S. typhi.

Certain aspects of the present disclosure are directed to an isolated nucleic acid comprising the nucleic acid sequence of SEQ ID NO: 6, SEQ ID NO: 7, SEQ ID NO: 8, SEQ ID NO: 9, or SEQ ID NO: 10, wherein the isolated nucleic acid is codon optimized for expression in Salmonella typhi Ty21a.

Certain aspects of the present disclosure provide a method of eliciting an immune response in a subject against a SARS-CoV-2 viral antigen or a modification thereof, the method comprising administering one or more doses of a composition or a vaccine disclosed herein to the subject. In some aspects, the composition or vaccine is administered to the mucosa of the subject. In some aspects, the composition or vaccine is administered orally or intranasally to the subject. In some aspects, the composition or vaccine is administered by inhalation. In some aspects, the composition or vaccine induces a humoral antibody response in the subject. In some aspects, the composition or vaccine induces a secretory IgA antibody response in the subject. In some aspects, the composition or vaccine induces a cellular T-cell response in the subject. In some aspects, the subject is a human subject. In some aspects, an immune response specific to a SARS-CoV-2 viral antigen is elicited in the subject by subsequent exposure of the subject to SARS-CoV-2. In some aspects, an immune response to a Salmonella typhi antigen is additionally elicited in the subject by subsequent exposure of the subject to pathogenic Salmonella typhi.

Certain aspects of the present disclosure provide a method of eliciting an immune response in a subject against a Salmonella typhi antigen, the method comprising administering one or more doses of a composition or a vaccine disclosed herein to the subject. In some aspects, the composition or vaccine is administered to the mucosa of the subject. In some aspects, the composition or vaccine is administered orally or intranasally to the subject. In some aspects, the composition or vaccine is administered by inhalation. In some aspects, the composition or vaccine induces a humoral antibody response in the subject. In some aspects, the composition or vaccine induces a secretory IgA antibody response in the subject. In some aspects, the composition or vaccine induces a cellular T-cell response in the subject. In some aspects, the subject is a human subject. In some aspects, an immune response specific to a Salmonella typhi antigen is elicited in the subject by subsequent exposure of the subject to pathogenic Salmonella typhi. In some aspects, an immune response to a SARS-CoV-2 antigen is additionally elicited in the subject by subsequent exposure of the subject to SARS-CoV-2.

Certain aspects of the present disclosure provide a method of treating, preventing, or reducing the incidence of COVID-19 or a SARS-CoV-2 viral infection in one or more subjects, the method comprising administering one or more doses of a composition or a vaccine disclosed herein to each subject. In some aspects, the composition or vaccine is administered to the mucosa of each subject. In some aspects, the composition or vaccine is administered orally or intranasally to each subject. In some aspects, the composition or vaccine is administered by inhalation. In some aspects, the composition or vaccine induces a humoral antibody response in the subject. In some aspects, the composition or vaccine induces a secretory IgA antibody response in the subject. In some aspects, the composition or vaccine induces a cellular T-cell response in the subject. In some aspects, the one or more subjects are human subjects.

Certain aspects of the present disclosure provide a method of treating, preventing, or reducing the incidence of typhoid fever in one or more subjects, the method comprising administering one or more doses of a composition or a vaccine disclosed herein to each subject. In some aspects, the composition or vaccine is administered to the mucosa of each subject. In some aspects, the composition or vaccine is administered orally or intranasally to each subject. In some aspects, the composition or vaccine is administered by inhalation. In some aspects, the composition or vaccine induces a humoral antibody response in the subject. In some aspects, the composition or vaccine induces a secretory IgA antibody response in the subject. In some aspects, the composition or vaccine induces a cellular T-cell response in the subject. In some aspects, the one or more subjects are human subjects.

Certain aspects of the present disclosure provide a method of treating, preventing, or reducing the incidence of both COVID-19 and typhoid fever in one or more subjects, the method comprising administering one or more doses of a composition or a vaccine disclosed herein to each subject. In some aspects, the composition or vaccine is administered to the mucosa of each subject. In some aspects, the composition or vaccine is administered orally or intranasally to each subject. In some aspects, the composition or vaccine is administered by inhalation. In some aspects, the composition or vaccine induces a humoral antibody response in the subject. In some aspects, the composition or vaccine induces a secretory IgA antibody response in the subject. In some aspects, the composition or vaccine induces a cellular T-cell response in the subject. In some aspects, the one or more subjects are human subjects.

### BRIEF DESCRIPTION OF THE DRAWINGS

FIG. 1 (FIG. 1) shows the structural components of a coronavirus viral particle (Alsaadi, E. A. J., and J. M. Jones, Future Virol. 14: 275-86 (2019)).

FIG. 2 (FIG. 2) shows the amino acid sequence of a modified SARS CoV-2 Spike glycoprotein ectodomain (eS) (SEQ ID NO: 1) (Wrapp, D., et al., Science 367: 1260-3 (2020)). Bolded and underlined=N-glycosylation sites; Solid box=RBD; Bolded and italicized=D614G substitution (Korber B., et al., Cell 182(4):812027 (2020)); Hashed box with short lines=GSAS substitution (for RRAR) at the furin cleavage site (residues 682-685); Hashed box with long lines=Proline substitutions at residues 986-987. Additions at the C-terminus: Hashed box with long and short lines=T4 fibritin trimerization motif.

FIGS. 3A-3B (FIGS. 3A-3B) show the structure (FIG. 3A) and amino acid sequence (FIG. 3B) of SARS-CoV2 M protein (SEQ ID NO: 2; GenBank Accession No. MN908947.3; https://COVID-19.uniprot.org/uniprotkb/PODTC5). The region corresponding to amino acids S108 to P123 of the M protein is a conserved region among SARS-CoV-2, SARS-CoV, Bat CoV, and MERS CoV (FIG. 3B, solid box).

FIGS. 4A-4B (FIGS. 4A-4B) show the structure (FIG. 4A) and amino acid sequence (FIG. 4B) of SARS-CoV-2 N protein (SEQ ID NO: 3; GenBank Accession No. MN908947.3; https://COVID-19.uniprot.org/uniprotkb/PODTC5). Intrinsically disordered regions (IDR) (a.a. 1-44; 182-247; 366-422); N terminal domain (NTD) (a.a. 45-181); Linker region (LKR) (182-247); C-terminal domain (CTD) (248-365). The charged SR rich (striated box) and the nuclear localization signal (NLS, solid box) motifs are shown (FIG. 1 of McBride, R., et al., Viruses 6(8), 2991-3018 (2014); Huang, Q., et al., Biochemistry (Mosc) 43: 6059-63 (2004)).

FIGS. 5A-5C (FIGS. 5A-5C) show the schematics of the Ty21a-eS (FIG. 5B) and Ty21a-eS-AR (FIG. 5C) constructs. Chromosomal integration of eS gene is achieved with the Ty21a-eS and Ty21a-eS-AR constructs (FIGS. 5A-5C). Secto=gene cassette representing the eS protein. Lpp=constitutive lpp promoter. Pₐᵣₐ=arabinose-inducible promoter.

FIGS. 6A-6C (FIGS. 6A-6C) show the schematics of the Ty21a-eS-M-N (FIG. 6B) and Ty21-eS-M-N-AR (FIG. 6C) constructs. Chromosomal integration of the SARS-CoV-2 spike protein ectodomain (eS), M, and N genes is achieved with the Ty21a-eS-M-N and Ty21a-eS-M-N-AR constructs (FIGS. 6A-6C).

FIGS. 7A-7B (FIGS. 7A-7B) show the plasmid expression of a recombinant SARS-CoV-2 eS in cell pellets (OD 0.15; FIG. 7A) and cell supernatant (OD 0.5; FIG. 7B). M=Marker, Ty21a, 2.5 ng Spike, 1-4=4 clones. Probed with rabbit anti-SARS-CoV-2/2019-nCo Spike.

FIGS. 8A-8B (FIGS. 8A-8B) show that a chromosomally integrated SARS-CoV-2 eS gene fragment expressed eS protein in both cell pellets (FIG. 8A) and cell supernatant (FIG. 8B) of the Ty21a-S-ecto clone. R=Ty21a with plasmid expressed Spike-ecto. X=chromosomal integrated Ty21a-S-ecto.

FIG. 9 (FIG. 9) shows the amino acid sequence of a modified SARS CoV-2 eS (SEQ ID NO: 5). Bolded=the SARS CoV-2 eS. Hashed boxes with long lines=spike modifications. Additions at the C-terminus: Hashed box with long and short lines=T4 fibritin trimerization motif; Hashed box with short lines=hemolysin A (HLYA) signal sequence.

FIG. 10 (FIG. 10) shows a schematic for the construction of a Ty21a-RBD-AR construct. As shown, the SARS-CoV-2 receptor binding domain (RBD) was fused to the HlyA secretion signal, and the Shigella sonnei acid resistance cassette was integrated into the Ty21a genome at the tviE locus. The schematic also shows locations for primers 27, 37, 89, 64, and 107, which were used in polymerase chain reaction (PCR) amplification.

FIGS. 11A-11C (FIG. 11A-11C) show confirmation of the integration via PCR. FIG. 11A shows PCR amplification using primers 27 and primer 107, which flank the homologous arms on both the 5′ and 3′ ends of the integration site. FIG. 11B shows PCR amplification using primer 27 (flanking the 5′ end of the homologous arm) and primer 37 (inside the insertion and downstream of KanR gene). FIG. 11C shows confirmation using primer 64 (downstream of the right homology arm) and primer 89 (inside the insertion and on the 5′ of the RBD).

### DETAILED DESCRIPTION OF THE DISCLOSURE

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. Thus, “about 10-20” means “about 10 to about 20.” In general, the term “about” can modify a numerical value above and below the stated value by a variance of, e.g., 10 percent, up or down (higher or lower).

The term “expression” as used herein refers to a process by which a polynucleotide produces a gene product, for example, an RNA or a polypeptide.

As used herein, the term “recombinant” includes the expression from genes made by genetic engineering or otherwise by laboratory manipulation.

“Biosynthetic” as used herein, means produced by a process whereby one or more substrates are converted to more complex products within a living organism or cell.

As used herein, “Deoxyribonucleic acid” or “DNA,” is a polynucleotide assembled in a particular sequence that encodes a polypeptide. DNA can include a promoter or secretion signal and/or other transcription or translation control elements operably associated with one or more coding regions. An operable association means that a coding region for a gene product, e.g., a polypeptide, is associated with one or more regulatory sequences in such a way as to place expression of the gene product under the influence or control of the regulatory sequence(s).

As used herein, “Nucleic acid” refers to any one or more nucleic acid segments, e.g., DNA fragments, present in a polynucleotide. Unless otherwise indicated, a particular nucleic acid sequence also implicitly encompasses conservatively modified variants thereof (e.g., degenerate codon substitutions, codon optimized sequences) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-8 (1985); Rossolini et al., Mol. Cell. Probes 8:91-8 (1994)).

“Sequence identity” as used herein refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multi-plied by 100 to yield the percentage of “sequence identity.” Percentage of “sequence identity” is determined by comparing two optimally aligned sequences over a comparison window. In order to optimally align sequences for comparison, the portion of a polynucleotide or polypeptide sequence in the comparison window may comprise additions or deletions termed gaps while the reference sequence is kept constant. An optimal alignment is that alignment which, even with gaps, produces the greatest possible number of “identical” positions between the reference and comparator sequences. The terms “sequence identity” and “identical” are used interchangeably herein. Accordingly, sequences sharing a percentage of “sequence identity” are understood to be that same percentage “identical.” In some embodiments, the percentage “sequence identity” between two sequences can be determined using the program “BLAST 2 Sequences” which was available from the National Center for Biotechnology Information, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-7 (1993)).

As used herein, a “gene” refers to a locus (or region) of DNA, which is made up of nucleotides that can that can be transcribed into RNA that in turn encodes a polypeptide.

As used herein, “gene region” refers to a location within chromosomal DNA that encodes one or more polypeptides of interest (e.g., an antigen).

“Gene system” as used herein refers to one or more genes that encode one or more polypeptides which when expressed in concert produce a desired effect.

“Variant,” as used herein, refers to a polypeptide that differs from the recited polypeptide due to amino acid substitutions, deletions, insertions, and/or modifications.

As used herein, “functional variant” means polypeptides that retain at least some of the properties of the corresponding wild-type polypeptide. For example, in some embodiments, the functional variant of an antigen retains at least 90%, at least 91%, at least 92%, at least 93%, at least 94%, at least 95%, at least 96%, at least 97%, at least 98%, at least 99%, or 100% antigenicity and/or protective immunity of the corresponding wild-type antigen.

As used herein, “transgenic” refers to an organism or cell that comprises a gene, a gene region, and/or a gene system that has been transferred to it by genetic engineering techniques.

“Integrated into” as used herein refers to incorporating a heterologous DNA (e.g., a gene, a gene region, and/or a gene system) into a chromosomal DNA.

“Heterologous” as used herein means from a different organism, cell type or species.

As used herein, “transformation,” “transfection,” and “transduction” refer to methods of transferring nucleic acid (i.e., a recombinant DNA) into a cell. The transferred nucleic acid can be introduced into a cell via an expression vector such as a plasmid, usually comprising components essential for selection, expression of target gene(s), and/or replication in the host cell.

“Stably expressed” as used herein refers to expression of a heterologous gene, a gene region and/or a gene system that has been integrated into chromosomal DNA in a fashion that is reproducible through multiple cell passages and/or under a broad range of physiologic conditions.

“Acid tolerance” as used herein refers to the ability of cells to remain viable at low pH, and after exposure to low pH such as passage through the stomach.

An “antigen” (also referred to as an immunogen) as used herein is a molecule capable of inducing an immune response in a host organism (e.g., a human) that is specific to that molecule.

“Immune response” as used herein means a response in a host organism, e.g., a human, to the introduction of an immunogen (e.g., a transgenic Ty21a of the application) generally characterized by, but not limited to, production of antibodies and/or T cells. In some embodiments, an immune response may be a cellular response such as induction or activation of CD4+ T cells or CD8+ T cells specific for an antigen, a humoral response of increased production of pathogen-specific antibodies, or both cellular and humoral responses.

“Vaccine” as used herein is a composition comprising an immunogenic agent (e.g., an immunogen or antigen) and a pharmaceutically acceptable diluent or carrier, optionally in combination with excipient, adjuvant and/or additive or protectant.

In certain embodiments, when a vaccine is administered to a subject, the immunogen (e.g., a transgenic Ty21a of the application) stimulates an immune response that will, upon subsequent exposure to an infectious agent, protect the subject from illness or mitigate the pathology, symptoms or clinical manifestations caused by that agent. In some embodiments, a therapeutic (treatment) vaccine is given after infection and is intended to reduce or arrest disease progression. In some embodiments, preventive (prophylactic) vaccine is intended to prevent initial infection or reduce the rate or burden of the infection.

“Carrier” as used herein refers to a substance suitable for pharmaceutical use. In some aspects, the carrier renders a composition (e.g., vaccine) suitable for pharmaceutical use. In some embodiments, the carrier is selected from the group consisting of water, PBS, saline, or any combination thereof. In another embodiment the carrier is selected from the group consisting of sucrose, ascorbic acid, amino acid mixture, lactose, magnesium stearate, or any combination thereof.

“Conferring protective immunity” refers to providing to a subject (i.e., an individual) or a human population (e.g., at least 10 subjects) the ability to generate an immune response to protect against a disease (e.g., COVID-19 or typhoid fever) caused by subsequent exposure to a pathogen (e.g., a bacteria or virus) such that the clinical manifestations, pathology, or symptoms of disease are reduced during subsequent exposure to the pathogen as compared to a non-treated subject, or such that the rate at which infection, or clinical manifestations, pathology, or symptoms of disease appear within a population are reduced, as compared to a non-treated population.

“Human population” as used herein refers to a group of humans which can be represented by a defined number of subjects, e.g., at least two subjects. For example, a human population comprises those individuals participating in a clinical trial, or individuals that have received a vaccine and are then challenged to assess protection.

“Dose” as used herein refers to a distinct administration event to a subject.

“Immunized” as used herein means sufficiently vaccinated to achieve a protective immune response.

As used herein, “stable,” with regard to a vaccine or pharmaceutical composition means the retention of potency for a given period of time. In some aspects, stability of a vaccine strain or a pharmaceutical composition comprising the vaccine strain is determined by assessing viability and/or residual water content over a period of time (e.g., at a certain temperature). In some aspects, viability is measured by growing colonies on an agar plate, counting the colony forming units (CFU), and scoring CFU per unit weight. In some aspects, viability is measured by removing a sample of the foam dried vaccine at one or more time points over a period of time, reconstituting the dried material in an equivalent volume of sterile water to the original pre-foam dried state (ml/mg), and quantitating CFU. In some aspects, CFU are quantitated by plating the reconstituted sample on tryptic soy agar and scoring for CFU/mg in the foam dried product after overnight incubation at 37° C. (Sim, B. K. L., et al., NPJ Vaccines 2:17 (2017)).

As used herein, the term “subject” includes any human or non-human animal. For example, the methods and compositions described herein can be used to treat, prevent, or reduce the incidence of a SARS-CoV-2 viral infection in a subject or to elicit an immune response in a subject against a SARS-CoV-2 viral antigen or a modification thereof. The term “non-human animal” includes all vertebrates, e.g., mammals and non-mammals, such as non-human primates, sheep, dog, cow, chickens, amphibians, reptiles, etc.

“Treat,” “treatment,” or “treating,” as used herein refers to, e.g., the reduction in severity of a disease or condition; the reduction in the duration of a condition course; the amelioration or elimination of one or more symptoms associated with a disease or condition; the provision of beneficial effects to a subject with a disease or condition, without necessarily curing the disease or condition.

“Pharmaceutical formulation” or “pharmaceutical composition,” as used herein, refers to a preparation which is in such form as to permit the biological activity of the active ingredient to be effective, and which contains no additional components which are unacceptably toxic to a subject to which the pharmaceutical formulation or composition would be administered. The pharmaceutical formulation or composition can be sterile. In some aspects, the pharmaceutical formulation or composition is suitable for therapeutic use in a human subject.

As used herein, the terms “ug” and “uM” are used interchangeably with “μg” and “μM,” respectively.

Any compositions or methods provided herein can be combined with one or more of any of the other compositions and methods provided herein.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

Various aspects described herein are described in further detail in the following subsections.

### II. Compositions

The present disclosure relates to the preparation and use of Salmonella typhi (S. typhi) Ty21a vectors to express foreign immunogens, e.g., one or more of the protein immunogens of the SARS-CoV-2 virus (e.g., S, eS, M, N), which have been stably integrated into the Ty21a chromosome. Such constructions provide bivalent or multivalent protection against both typhoid fever (the disease caused by S. typhi per se) as well as the disease associated with said foreign immunogen (e.g., COVID-19).

A challenge with the spike protein of SARS-CoV-2 is that it contains multiple N-glycosylation sites, positioned in the ectodomain. Therefore, when expressing foreign glycoproteins, e.g., in bacteria, they may fail to be secreted and rather form insoluble aggregates in the intracellular matrix. For example, Chuck et al. (Virus Genes 38:1-9 (2009)) had compared the expression and purification of six different fragments of the SARS CoV spike in E. coli and Pichia pastoris yeast. Chuck et al. found that the spike fragments expressed in E. coli formed aggregates and were insoluble whereas expression in the P. pastoris resulted in a significantly higher protein yields, purity and solubility. In certain aspects, the present disclosure provides for the expression of SARS-CoV spike proteins using bacterial Ty21a as disclosed with reduced or no aggregation of the expressed spike proteins (e.g., one or more SARS-CoV-2 viral antigens).

In some aspects, the S. typhi Ty21a vector used to express foreign immunogens is a transgenic S. typhi Ty21a comprising a chromosome with one or more heterologous nucleic acid regions (e.g., one or more biosynthetic gene regions), wherein the heterologous nucleic acid regions encode one or more SARS-CoV-2 viral antigens and are integrated into the S. typhi Ty21a chromosome, and wherein the transgenic S. typhi Ty21a stably expresses the one or more SARS-CoV-2 viral antigens.

In some aspects, the one or more heterologous nucleic acid regions of the transgenic S. typhi Ty21a vector encode a portion or all of one or more SARS-CoV-2 structural proteins (e.g., the spike (S) protein, the spike protein ectodomain (eS), the membrane (M) protein, and/or the nucleocapsid (N) protein), either naturally occurring or modified. In some aspects, the one or more heterologous nucleic acid regions are recombineered into the chromosome of Ty21a to stably express these immunogens as vaccine candidates.

In some aspects, the one or more heterologous nucleic acid regions of the transgenic S. typhi Ty21a vector comprise: (a) a nucleic acid sequence encoding a SARS-CoV-2 spike (S) protein or one or more antigenic fragments thereof, (b) a nucleic acid sequence encoding one or more antigens retaining at least 70% amino acid residue homology to a native SARS-CoV-2 S protein antigen, (c) a nucleic acid sequence encoding a SARS-CoV-2 spike protein ectodomain (eS) or one or more antigenic fragments thereof, (d) a nucleic acid encoding one or more antigens retaining at least 70% amino acid residue homology to a native SARS-CoV-2 eS antigen, (e) a nucleic acid sequence encoding a SARS-CoV2 membrane (M) protein or one or more antigenic fragments thereof, (f) a nucleic acid sequence encoding one or more antigens retaining at least 70% amino acid residue homology to a native SARS-CoV-2 M protein antigen, (g) a nucleic acid sequence encoding a SARS-CoV2 nucleocapsid (N) protein or one or more antigenic fragments thereof, (h) a nucleic acid sequence encoding one or more antigens retaining at least 70% amino acid residue homology to a native SARS-CoV-2 N protein antigen, or (i) any combination thereof.

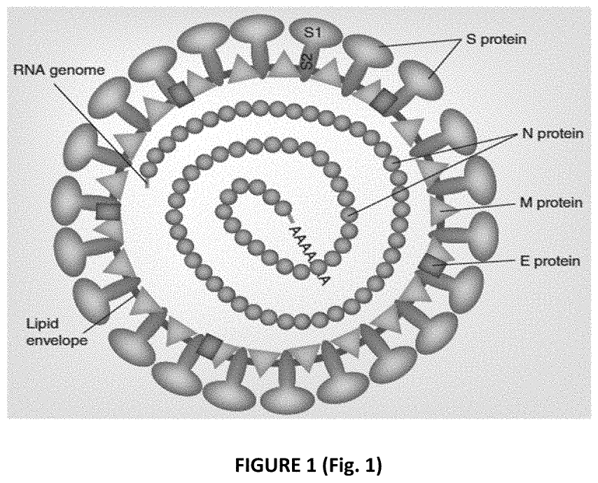

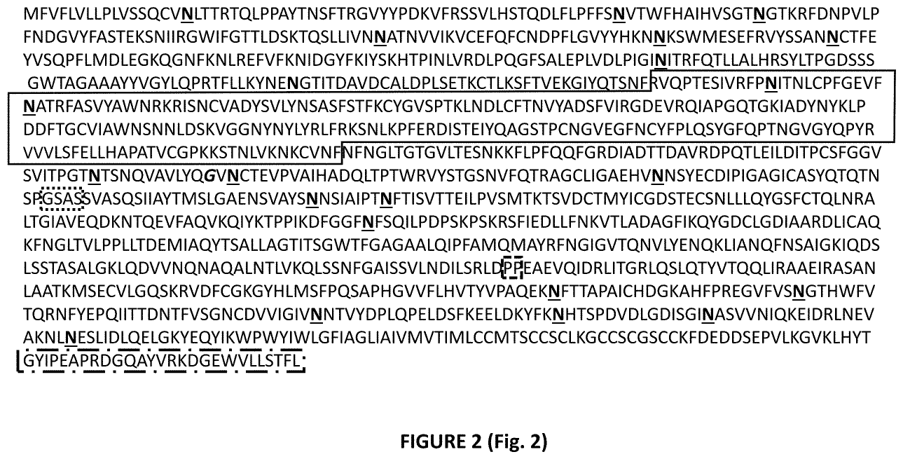

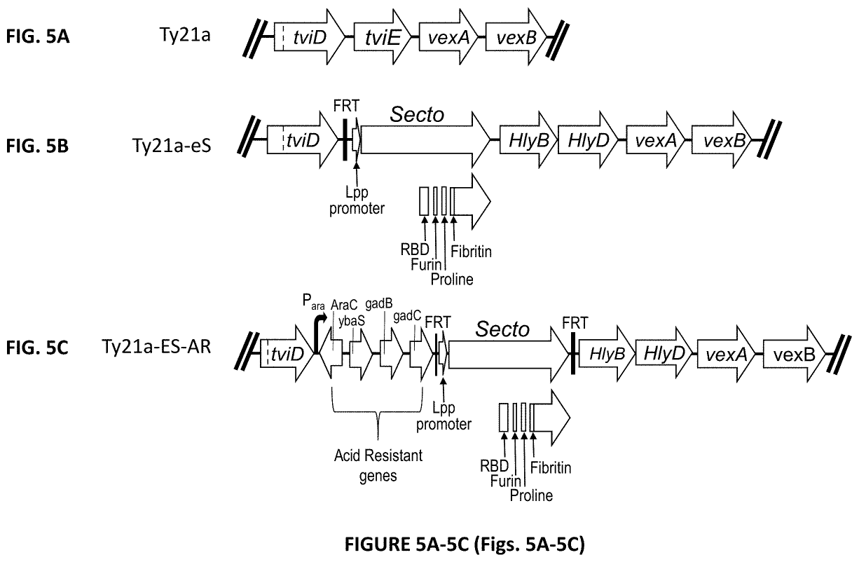

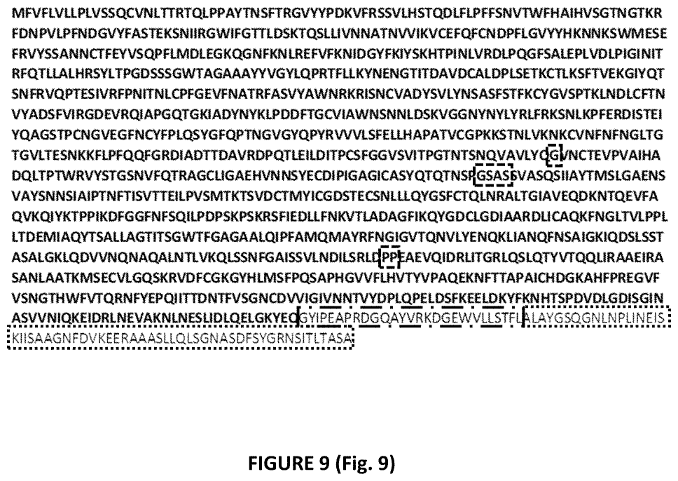

Ty21a encoding a modified SARS-CoV-2 eS protein (Ty21a-eS). In some aspects, the one or more heterologous nucleic acid regions of the transgenic S. typhi Ty21a vector encode a modified SARS-CoV-2 eS protein. The SARS-CoV-2 S protein is a large trimeric class I fusion protein that exists in a metastable prefusion conformation that undergoes a substantial structural rearrangement to fuse the viral membrane with the host cell membrane (Li, F., Annu Rev Viral. 3: 237-61 (2016); Bosch B. J., et al., J Virol. 77: 8801-11 (2003)) of its receptor ACE2 on target cells (high concentrations on epithelial cells of the oral cavity) (Xu H., et al., Int J Oral Sci. 12: 8 (2020)). The S protein consists of two subunits, S1 at the N-terminus providing the receptor binding function and S2 at the C-terminus providing fusion activity (FIG. 1) (Belouzard, S., et al., Viruses 4: 1011-33 (2012)). For mediation of membrane fusion, cleavage of S/S2 must occur. High infectivity and transmissibility of SARS-COV-2 as compared to SARS-COV has been linked to cleavage efficiency (Coutard, B., et al., Antiviral Res. 176: 104742 (2020)). In some aspects, the modified eS comprises a substitution of residues GSAS for RRAR at the furin cleavage site (residues 682-685), for improved accessibility and cleavage (Wrapp, D., et al., Science 367: 1260-3 (2020)). In some aspects, Foldon, the T4 fibritin trimerization domain that triggers obligatory trimerization (SEQ ID NO: 12) (Meier, S, et al., J Mol Biol. 344: 1051-69 (2004)), is included in the modified eS with the intent of proper trimerization as shown in other systems (Lu, Y., et al., Proc Natl Acad Sci USA 111: 125-30 (2014)). Thus, in some aspects, the eS encoded by the one or more heterologous nucleic acid regions contains modifications that stabilize the eS in its prefusion structure, thereby increasing eS expression, secretion, and immunogenicity (Wrapp, D., et al., Science 367: 1260-3 (2020); Pallesen, J., Proc Natl Acad Sci USA 114: E7348-E7357 (2017)) (FIG. 2). In some aspects, the modified eS comprises a D614G substitution (Korber B., et al., Cell 182(4):812027 (2020); Grubaugh, N. D., et al., Cell 182(4):794-5 (2020)). There are data on ‘HexaPro’ which show 4 substitutions of prolines (F817P, A892P, A899P, A942P) result in greater expression and stability (Hsieh C-L, et al., bioRxiv: 2020.2005.2030.125484, 2020)). In some aspects, the modified eS comprises F817P, A892P, A899P, and A942P substitutions. In some aspects, the modified eS comprises D614G, F817P, A892P, A899P, and A942P substitutions. In some aspects, the modified eS comprises proline substitutions at residues 986-987. In some aspects, the modified eS comprises a HLYA (SEQ ID NO: 13). In some aspects, the modified eS comprises the amino acids shown in FIG. 2 (SEQ ID NO: 1) or FIG. 9 (SEQ ID NO: 5). Ty21a-eS (SEQ ID NO: 6), which expresses a modified eS protein, is constructed and shown in FIG. 5B. Ty21a-eS-AR (SEQ ID NO: 8), which expresses a modified eS protein and comprises a heterologous AR gene cassette, is constructed and shown in FIG. 5C. The sequences described herein are disclosed in Table 7.

Ty21a encoding a modified SARS-CoV-2 Receptor Binding Domain (RBD) (Ty21a-RBD). In some aspects, the one or more heterologous nucleic acid regions of the transgenic S. typhi Ty21a vector encode a modified SARS-CoV-2 RBD. The RBD is part of the ectodomain of the Spike (S) protein. In some aspects, the modified RBD comprises a D614G substitution. In some aspects, the modified RBD comprises a substitution of residues GSAS for RRAR at the furin cleavage site (residues 682-685). In some aspects, the modified RBD comprises proline substitutions at residues 986-987. In some aspects, the modified RBD comprises T4 fibritin trimerization motif at the C-terminus (SEQ ID NO: 12). In some aspects, the modified RBD comprises a HLYA (SEQ ID NO: 13). In some aspects, the modified SARS-CoV-2 RBD comprises the amino acid sequence of SEQ ID NO: 11 (solid box of FIG. 2).

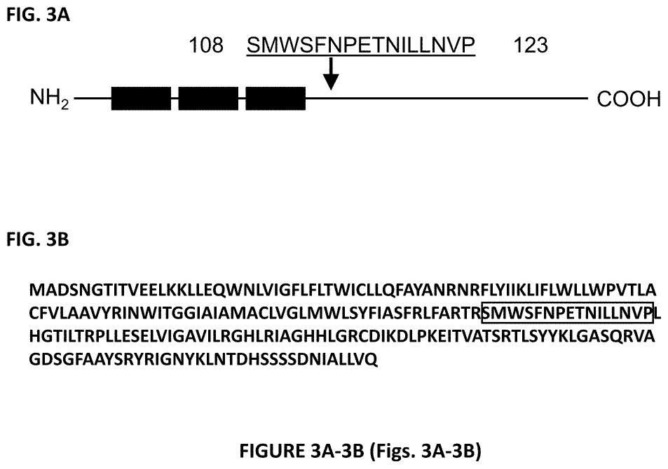

Ty21a encoding a SARS-CoV-2 Membrane (M) protein (Ty21a-M). In some aspects, the one or more heterologous nucleic acid regions of the transgenic S. typhi Ty21a vector encode the entire SARS-CoV-2 M protein. The M protein is a type III transmembrane protein and is the most abundant protein in the CoV particle and functions in virus assembly at the budding site. It is composed of three parts: a short N-terminal domain situated outside the virion membrane, three transmembrane domains, and a carboxy-terminal domain inside the particle (Hogue, B. G., and Machamer, C. E., 2008. Coronavirus structural proteins and virus assembly. In Nidoviruses, ed. Perlman, S., Gallagher, T, and Snijder, E., pp. 179-200. Washington, D.C.: ASM Press; Ujike, M., and F. Taguchi, Viruses 7:1700-25 (2015)). An amphipathic region at the end of the 3^{rd} transmembrane domain is conserved in almost all Coronaviridae (Arndt, A. L., et al., J Virol. 84: 11418-28 (2010)). In some aspects, the SARS-CoV-2 M protein comprises the amino acids shown in FIG. 3B (SEQ ID NO: 2). The SARS-CoV2 M protein is 222 amino acids in length. Ty21a-M, which expresses the entire M protein and includes all M protein epitopes and domains, is shown in FIGS. 3A-3B (Grifoni, A., et al., Cell 181:1489-1501 (2020); Arndt, A. L., et al., J Virol. 84: 11418-28 (2010); Grifoni, A., et al., Cell Host Microbe 27: 671-80 (2020)).

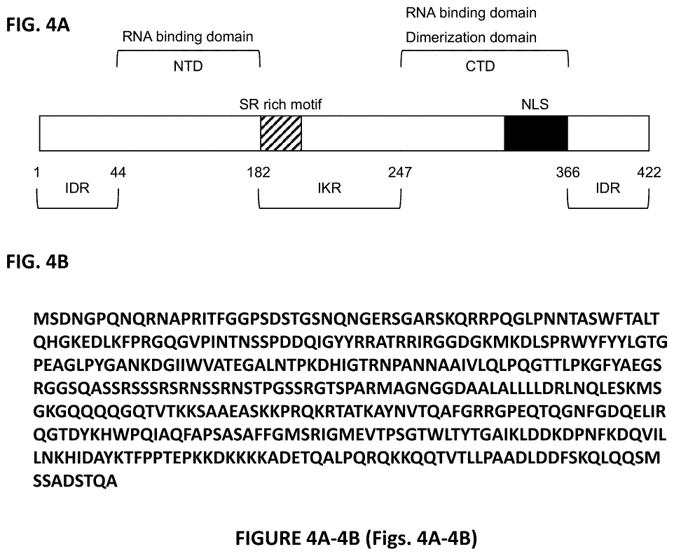

Ty21 encoding a SARS-CoV-2 Neucleocapsid (N) protein (Ty21a-N). In some aspects, the one or more heterologous nucleic acid regions of the transgenic S. typhi Ty21a vector encode the entire SARS-CoV-2 N protein. The N protein, a major structural protein in the virion, chaperones and protects the viral RNA genome (Sturman, L. S., et al., J Virol. 33:449-62 (1980)). In some aspects, the SARS-CoV-2 N protein comprises the amino acids shown in FIG. 4B (SEQ ID NO: 3). Ty21a-N, which expresses the entire N protein and includes all N protein epitopes and domains, is constructed and shown in FIGS. 4A-4B (Grifoni, A., et al., Cell Host Microbe 27: 671-80 (2020)).

Ty21 encoding a SARS-CoV-2 M protein and a SARS-CoV-2 N (Ty21a-M-N). In some aspects, the one or more heterologous nucleic acid regions of the transgenic S. typhi Ty21a vector encode the entire SARS-CoV-2 M protein and the entire SARS-CoV-2 N protein. In some aspects, the SARS-CoV-2 M protein comprises the amino acids shown in FIG. 3B (SEQ ID NO: 2, and the SARS-CoV-2 N protein comprises the amino acids shown in FIG. 4B (SEQ ID NO: 3). Ty21a-M-N(SEQ ID NO: 7), which expresses the entire M and N proteins and includes all epitopes and domains of the M and N proteins, is constructed and shown in FIGS. 4A-4B (Grifoni, A., et al., Cell Host Microbe 27: 671-80 (2020)).

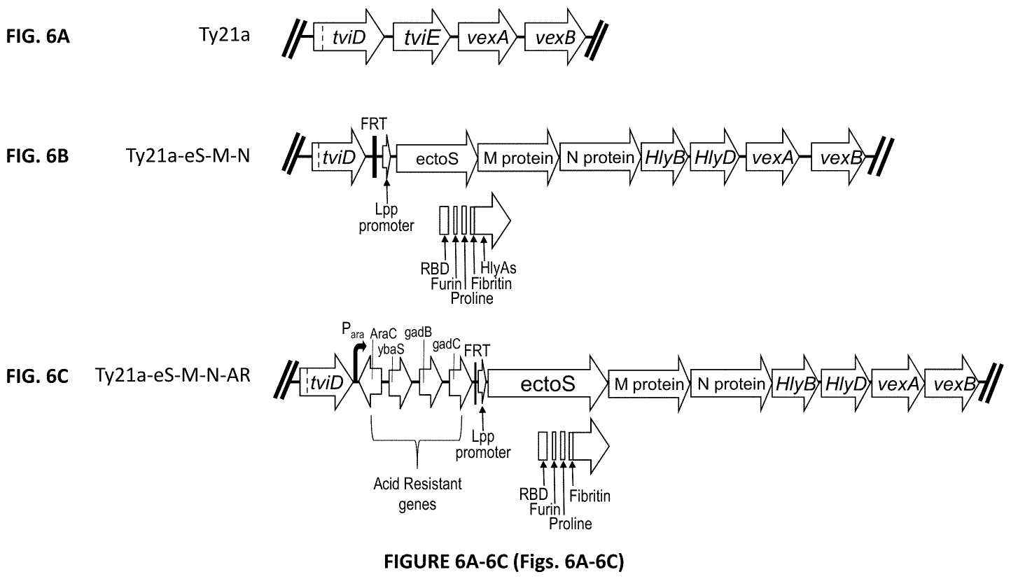

Ty21 encoding a modified SARS-CoV-2 eS protein, a SARS-CoV-2 M protein, and a SARS-CoV-2 N protein (Ty21a-eS-M-N). In some aspects, the one or more heterologous nucleic acid regions of the transgenic S. typhi Ty21a vector encode a modified SARS-CoV-2 eS protein, a full-length SARS-CoV-2 M protein, and a full-length SARS-CoV-2 N protein. Ty21a-eS-M-N, which expresses a modified eS protein, the entire M protein, and the entire N protein, is constructed and shown in FIG. 6B.

In some aspects, the transgenic S. typhi Ty21a vectors disclosed herein further comprise a heterologous acid resistance (AR) gene cassette (e.g., a heterologous AR biosynthetic gene system) integrated into the S. typhi Ty21a chromosome. Ty21a-eS-AR (SEQ ID NO: 8), which expresses a modified eS protein, the entire M protein, and the entire N protein and comprises a heterologous AR gene cassette, is constructed and shown in FIG. 5C. Ty21a-M-N-AR (SEQ ID NO: 9), which expresses the entire M protein, and the entire N protein and comprises a heterologous AR gene cassette, is constructed. Ty21a-es-M-N-AR (SEQ ID NO: 10), which expresses a modified eS protein, the entire M protein, and the entire N protein and comprises a heterologous AR gene cassette, is constructed and shown in FIG. 6C.

Integration of a heterologous AR gene cassette provides acid stability and enhances viability of recombinant Ty21a as it passes through the stomach where conditions are acidic, thereby providing for more stable gene expression. This can also eliminate the need for gelatin capsules or liquid formulations and provides temperature stabilization and extended shelf life. Non-limiting examples of heterologous AR biosynthetic gene system are disclosed in U.S. Pat. No. 10,695,415, which is incorporated by reference herein in its entirety, and include Shigella glutamine-glutamate AR gene systems. In some aspects, the immunogenicity of the S. typhi Ty21a vectors disclosed herein may be enhanced by improving its ability to withstand low pH passage through the stomach by incorporating a Shigella glutamine-glutamate AR gene system.

In some aspects, the heterologous AR gene cassette comprises a gene selected from the group consisting of: a YbaS gene (SEQ ID NO: 15) or a fragment thereof, a GadB gene (SEQ ID NO: 17) or fragment thereof, a GadC gene (SEQ ID NO: 19) or fragment thereof, a GadA gene (SEQ ID NO: 22) or fragment thereof, and any combination thereof. In some aspects, the heterologous AR gene cassette comprises a sequence having at least 80%, 85%, 90%, 95%, or 100% sequence identity to one or more nucleic acid sequences selected from: SEQ ID NO: 15, SEQ ID NO: 17, SEQ ID NO: 19, SEQ ID NO: 20, and SEQ ID NO: 22. In some aspects, the transgenic S. typhi Ty21a vectors comprising the heterologous AR gene cassette are more acid stable at pH 2.5 than Salmonella typhi Ty21a without the integrated AR gene cassette.

In some aspects, the S. typhi Ty21a vectors of the present disclosure are formulated as a composition or vaccine. In some aspects, the composition or vaccine comprises a carrier that renders the composition or vaccine suitable for pharmaceutical use. In some aspects, the composition or vaccine is suitable for oral or inhaled administration (e.g., nasal aerosol administration). In some aspects, the S. typhi Ty21a vectors of the present disclosure are formulated as a composition or vaccine for oral or inhaled administration (e.g., nasal aerosol administration) and comprise an integrated heterologous AR cassette. In some aspects, the S. typhi Ty21a vectors of the present disclosure are formulated as a composition or vaccine for oral or inhaled administration (e.g., nasal aerosol administration) without said integration of a heterologous AR cassette.

In some aspects, the vaccine is protective against COVID-19. In some aspects, the vaccine is protective against typhoid fever. In some aspects, the vaccine is protective against COVID-19 and typhoid fever.

In some aspects, administration of the vaccine to one or more individuals or a human population reduces the incidence of COVID-19 in the one or more individuals or the human population when subsequently exposed to pathogenic SARS-CoV-2. In some aspects, administration of the vaccine to one or more individuals or a human population reduces the incidence of typhoid fever in the one or more individuals or the human population when subsequently exposed to pathogenic S. typhi. In some aspects, administration of the vaccine to one or more individuals or a human population reduces the incidence of both pathogenic SARS-CoV-2 and typhoid fever in the one or more individuals or the human population when subsequently exposed to pathogenic SARS-CoV-2 and pathogenic S. typhi.

In some aspects, the vaccine is foam dried for stabilization at room temperature. In some aspects, the vaccine is at least 90% stable, at least 95% stable, at least 99% stable, or 100% stable at room temperature for at least one month. In some aspects, the vaccine is at least 40% stable, at least 50% stable, at least 60% stable, at least 70% stable, at least 80% stable, at least 90% stable, at least 95% stable, at least 99% stable, or 100% stable at room temperature for at least one year. In some aspects, the vaccine is at least 40% stable, at least 50% stable, at least 60% stable, at least 70% stable, at least 80% stable, at least 90% stable, at least 95% stable, at least 99% stable, or 100% stable at room temperature for at least two years. In some aspects, the vaccine has a shelf-life of 6-24 months, 1-2 years, 1-5 years, 1-10 years, or 5-10 years at 4° C. In some aspects, stability of a foam dried vaccine is determined by assessing viability and/or residual water content over a period of time (e.g., for at least one year at room temperature or for 6-24 months, 1-2 years, 1-5 years, 1-10 years, or 5-10 years at 4° C.). In some aspects, viability is measured by growing colonies on an agar plate, counting the colony forming units (CFU), and scoring CFU per unit weight. In some aspects, viability is measured by removing a sample of the foam dried vaccine at one or more time points over a period of time, reconstituting the dried material in an equivalent volume of sterile water to the original pre-foam dried state (ml/mg), and quantitating CFU. In some aspects, CFU are quantitated by plating the reconstituted sample on tryptic soy agar and scoring for CFU/mg in the foam dried product after overnight incubation at 37° C. (Sim, B. K. L., et al., NPJ Vaccines 2:17 (2017)).

The disclosure further provides an isolated nucleic acid comprising the nucleic acid sequence of SEQ ID NO: 6 (Ty21a-eS construct), SEQ ID NO: 7 (Ty21a-M-N construct), SEQ ID NO: 8 (Ty21a-eS-AR construct), SEQ ID NO: 9 (Ty21a-M-N-AR construct), or SEQ ID NO: 10 (Ty21a-eS-M-N-AR construct), wherein the isolated nucleic acid is codon optimized for expression in Salmonella typhi Ty21.

### III. Methods of Use

The disclosure provides methods of eliciting an immune response in a subject against a SARS-CoV-2 viral antigen or a modification thereof comprising administering one or more doses of a composition or vaccine disclosed herein to the subject. In some aspects, an immune response specific to a SARS-CoV-2 antigen is elicited in the subject by subsequent exposure of the subject to SARS-CoV-2. In some aspects, an immune response to a Salmonella typhi antigen is additionally elicited in the subject by subsequent exposure of the subject to pathogenic Salmonella typhi.

The disclosure further provides methods of eliciting an immune response in a subject against a Salmonella typhi antigen comprising administering one or more doses of a composition or a vaccine disclosed herein to the subject. In some aspects, an immune response specific to a Salmonella typhi antigen is elicited in the subject by subsequent exposure of the subject to pathogenic Salmonella typhi. In some aspects, an immune response to a SARS-CoV-2 antigen is additionally elicited in the subject by subsequent exposure of the subject to SARS-CoV-2.

The disclosure further provides methods of eliciting an immune response in a subject against both a SARS-CoV-2 viral antigen or a modification thereof and a Salmonella typhi antigen comprising administering one or more doses of a composition or vaccine disclosed herein to the subject. In some aspects, an immune response to a SARS-CoV-2 antigen is elicited in the subject by subsequent exposure of the subject to SARS-CoV-2, and wherein an immune response specific to a Salmonella typhi antigen is elicited in the subject by subsequent exposure of the subject to pathogenic Salmonella typhi.

The disclosure further provides methods of treating, preventing, or reducing the incidence of COVID-19 or a SARS-CoV-2 viral infection in one or more subjects comprising administering one or more doses of a composition or a vaccine disclosed herein to each subject.

The disclosure further provides methods of treating, preventing, or reducing the incidence of typhoid fever in one or more subjects comprising administering one or more doses of a composition or vaccine disclosed herein to each subject.

The disclosure further provides methods of treating, preventing, or reducing the incidence of both COVID-19 and typhoid fever in one or more subjects comprising administering one or more doses of a composition or vaccine disclosed herein to each subject.

In some aspects, the composition or vaccine induces a humoral antibody response in the subject. In some aspects, the composition or vaccine induces a secretory IgA antibody response in the subject. In some aspects, the composition or vaccine induces a cellular T-cell response in the subject.

In some aspects, the administration route is oral or nasal. In some aspects, the administration (e.g., immunization) and/or infection route is oral (per os). In some aspects, the administration (e.g., immunization) route is nasal. In some aspects, the composition or vaccine is administered by inhalation. The major barrier for a live oral vaccine is the extreme low pH the vaccine encounters in the stomach. Salmonella does not survive well under conditions <pH 3, while most E. coli strains and Shigella spp. can maintain viability in stomach for several hours (Lin, J., et al., J Bacteriol. 177(14):4097-104 (1995); Gorden, J. and P. L. Small, Infect Immun, 61(1):364-7 (1993)). The ability of Shigella, and the inability of Salmonella, to survive at low pH may partially explain why only 10-100 Shigella cells are sufficient to cause infection, while the infective dose for Salmonella spp. ranges at ˜10⁵ CFU (U.S. Pat. No. 10,695,415). Salmonella expresses acid tolerance response (ATR) genes in response to moderately low pH, which protect the cells from acid challenge as low as pH 3 (Lin, J., et al. (1995); Audia, J. P., et al., Int J Med Microbiol. 291(2):97-106 (2001); Foster, J. W., J Bacteriol. 173(21):6896-902 (1991); Foster, J. W., J Bacteriol, 175(7):1981-7 (1993); Foster, J. W., Crit Rev Microbiol, 21(4):215-37 (1995)). Ty21a inherited an rpoS mutation from its parental strain Ty2 (Robbe-Saule, V. and F. Norel, FEMS Microbiol Lett. 170(1):141-3 (1999)), and carries other less well defined mutations from the random mutagenesis process during strain attenuation (Germanier, R. and E. Fuer, J Infect Dis. 131(5):553-8 (1975)). Perhaps because of these mutations, Ty21a develops a poor ATR response and is particularly sensitive to low pH (Hone, D. M., et al., Vaccine 12(10):895-8 (1994)). However, Ty21a viability is important for vaccine efficacy, as a previous report demonstrated that, when administered orally, live Ty21a elicited stronger and longer lasting immune responses in humans than killed Ty21a (Kantele, A., et al., Microb Pathog. 10(2):117-26 (1991)). To facilitate the journey from mouth to ileum without being eliminated in the gastric acid environment, Ty21a is presently placed in enteric-coated capsules that withstand gastric low pH. Additionally, when administered as a liquid with a buffer, Ty21a was more protective (Levine, M. M., et al., Vaccine 17 Suppl 2:S22-7 (1999)). On the other hand, capsules are child-unfriendly and adversely impact compliance. Also, the Ty21a liquid formulation has been commercially unsuccessful, in part because it is cumbersome.

To obviate the need for special capsules or liquid formulation in buffer, increase bioavailability, reduce dosage requirements, and increase immunogenicity, Ty21a has been rendered acid tolerant (also referred to as acid resistant). There are 5 bacterial acid resistance (AR) pathways, which utilize excess protons to decarboxylate a specific amino acid (e.g. aspartic acid, phenylalanine, lysine, or glutamic acid), and an antiporter that transports the decarboxylated product extracellularly (Waterman, S. R. and P. L. Small, FEMS Microbiology Letters 225(1):155-60 (2003); Bhagwat, A. A. and M. Bhagwat, FEMS Microbiology Letters 234(1):139-47 (2004)). The ability of E. coli, Shigella, Listeria monocytogenes and Lactococcus lactis to withstand extreme acidic pH (below pH 2.5) primarily relies on the most potent AR system, AR2, also known as the glutamate-dependent acid resistance (GDAR) pathway (Lin, J., et al. (1995); Lin, J., et al., Appl Environ Microbiol. 62(9):3094-100 (1996)). AR2 consists of the enzyme glutamate decarboxylase (GAD), encoded by the homologous genes gadA and gadB, and a membrane bound antiporter, encoded by the gene gadC. The two GAD isoforms, GadA and GadB, consume an intracellular proton to decarboxylate glutamate, producing γ-amino butyric acid (GABA) and CO₂ (Audia, J. P., et al. (2001); Brenneman, K. E., et al., J Bacteriol. 195(13):3062-72 (2013); De Biase, D. and E. Pennacchietti, Mol Microbiol. 86(4):770-86 (2012); Merrell, D. S. and A. Camilli, Curr Opin Microbiol. 5(1):51-5 (2002); Zhao, B. and W. A. Houry, Biochem Cell Biol. 88(2):301-14 (2010)), while GadC pumps the substrate (glutamate) and product (GABA) in and out of the cell (U.S. Pat. No. 10,695,415; De Biase, D. and E. Pennacchietti, Mol Microbiol, 86(4):770-86 (2012)). Genes of the two GAD isoforms are located in two distinct chromosomal loci, with gadB and gadC transcribed as a dicistronic operon, and gadA from a separate gene locus (De Biase, D. and E. Pennacchietti (2012); Hersh, B. M., et al., J Bacteriol. 178(13):3978-81 (1996); Smith, D. K., et al., J Bacteriol. 174(18):5820-6 (1992)). GadA and GadB are highly similar, with sequence identity of 96.5% at nucleotide level and 98.7% at protein level. Deletion of either gadA or gadB does not affect the cell's AR ability, suggesting these two isozymes are functionally redundant. Newly discovered glutamine-dependent AR system in E. coli (Lu, P., et al., Cell Res. 23(5):635-44 (2013)) and Lactobacillus reuteri (Datsenko, K. A. and B. L. Wanner, Proc Natl Acad Sci USA 97(12):6640-5 (2000)) have been reported. In E. coli, a previously uncharacterized bacterial glutaminase A, encoded by the gene ybaS, converts glutamine into glutamate in acidic conditions and releases an ammonium, neutralizing an intracellular proton (Lu, P., et al. (2013)). Interestingly, the antiporter responsible for substrate-product transportation across cell membrane is also GadC (Lu, P., et al. (2013)), consistent with the broad substrate specificity of GadC in vitro (Ma, D., et al., Nature 483(7391):632-6 (2012)). Because the product of glutaminase is precisely the substrate of GAD, the YbaS-GadC and GAD-GadC systems work in concert to convert a glutamine molecule into GABA, neutralizing two protons in the cell (U.S. Pat. No. 10,695,415; Lu, P., et al. (2013)), thereby doubling the proton-reducing capacity of the system. This concerted AR system functions more efficiently than the GAD-GadC system alone.

In addition to providing multivalent protection against both typhoid fever and SARS-CoV-2 antigens, in some embodiments, integration of an acid resistance cassette eliminates the need for gelatin capsules or liquid formulations and provides temperature stabilization and extended shelf life.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooks, Antisense drug Technology: Principles, strategies and applications, 2nd Ed. CRC Press (2007) and in Ausubel et al. (1989) Current Protocols in Molecular Biology (John Wiley and Sons, Baltimore, Md.).

All of the references cited above, as well as all references cited herein and the amino acid or nucleotide sequences (e.g., GenBank numbers and/or Uniprot numbers), are incorporated herein by reference in their entireties.

The following examples are offered by way of illustration and not by way of limitation.

### IV. Examples

### IV.A. Example 1: Development of Transgenic Ty21a Strains

The eS sequence (FIG. 2) was synthesized with intended modifications in plasmid puc57 separately (Genewiz, South Plainfield, N.J.) for recombineering into the Ty21 chromosome at the tviE locus (Ty21a-eS; FIGS. 5A-5B). The k-red recombineering technology (Datsenko, K. A., and B. L. Wanner, Proc Natl Acad Sci USA 97:6640-5 (2000); Dharmasena, M. N., et al., Int J Med Microbiol. 303:105-13 (2013)) was used along with methods previously described (Wu, Y., et al., J Infect Dis. 215: 259-68 (2017); Sim, B. K. L., et al., Vaccine 38: 5123-30 (2020). The constitutive lpp promoter (Lpp-outer membrane lipoprotein) (Inouye, S. and M. Inouye, Nucleic Acids Res. 13:3101-10 (1985)) was used to provide high expression, and the hemolysin A type 1 secretion system (Hly) was used (Thomas S., et al., Biochimica et Biophysica Acta (BBA)—Molecular Cell Research 1843:1629-41 (2014)) as the secretion signal (FIG. 5B). Creation of the acid stable counterpart candidates (Ty21a-eS-AR; FIG. 5C) included the concerted glutaminase-GAD AR systems under the control of AraC the transcriptional regulator for ara operon and the arabinose-inducible promoter (Pₐᵣₐ) (Wu, Y., et al., J Infect Dis. 215: 259-68 (2017); Dorman, C. J., EcoSal Plus 1(1), DOI: 10.1128/ecosalplus.8.9.3 (2004)) (FIG. 5C). A seed bank (20 glycerol stabilate vials stored at −80° C.) was generated for each of the transgenic Ty21a-eS and Ty21a-eS-AR strains.

Ty21a-RBD, Ty21a-RBD-AR, Ty21a-M, Ty21a-M-AR, Ty21a-N, Ty21a-N-AR, Ty21a-M-N, Ty21a-M-N-AR, Ty21a-eS-M-N (FIG. 6B), and Ty21a-eS-M-N-AR (FIG. 6C) constructs were synthesized for recombineering into the Ty21 chromosome at the tviE locus (Ty21a-eS; FIGS. 5A and 6A) using an approach similar to the approach described above for the Ty21a-eS and Ty21a-eS-AR constructs. A seed bank (20 glycerol stabilate vials stored at −80° C.) was generated for each of the Ty21a-RBD, Ty21a-RBD-AR, Ty21a-M, Ty21a-M-AR, Ty21a-N, Ty21a-N-AR, Ty21a-M-N, Ty21a-M-N-AR, Ty21a-eS-M-N, and Ty21a-eS-M-N-AR transgenic strains.

### IV.B. Example 2: Characterization of Transgenic Ty21a Strains

i) Secreted expression of recombinant eS from transgenic Ty21a-eS and Ty21a-eS-AR strains was verified by immunoblot using antibodies against S protein and RBD (ProSci, Poway, Calif.); ii) Binding capability of recombinant eS was assessed in ELISAs with binding to recombinant ACE2 as capture antigen (Acro Biosystems, Newark, Del.); iii) Strains were characterized by total genome analysis; iv) Expression stability of chromosomally integrated eS gene fragment was confirmed by characterizing after culturing through 200 generations of growth; v) Strains were microbiologically characterized by analytical profile index (API) analysis-20E Enteric ID system to discriminate for family Enterobacteriaceae; vi) Vaccine strains for known Ty21a phenotypes (galactose-induced cell lysis, isoleucine-valine deficiency, cysteine-deficiency, tryptophan-deficiency, weak H₂S production) were assessed.

Transgenic Ty21a-RBD, Ty21a-RBD-AR, Ty21a-M, Ty21a-M-AR, Ty21a-N, Ty21a-N-AR, Ty21a-M-N, Ty21a-M-N-AR, Ty21a-eS-M-N, and Ty21a-eS-M-N-AR strains were also characterized for i) secreted expression of recombinant eS or RBD, ii) binding capability of recombinant eS, iii) by total genome analysis, iv) expression stability of chromosomally integrated eS, M, and/or N gene fragments, v) API to discriminate for family Enterobacteriaceae and/or vi) known Ty21a phenotypes (galactose-induced cell lysis, isoleucine-valine deficiency, cysteine-deficiency, tryptophan-deficiency, weak H₂S production).

### IV.C. Example 3: Assessment of Proper Folding of eS or RBD

The RayBio COVID-19 Spike-ACE2 binding assay II (RayBio, Peachtree Corners, Ga.) was used to characterize the binding capability of secreted recombinant eS or RBD to the Angiotensin I Converting Enzyme 2 (ACE2) receptor complex. The assay used a 96-well plate coated with recombinantly-expressed human ACE2 protein. The supernatant of Ty21a-eS, Ty21a-eS-AR, Ty21a-RBD, Ty21a-RBD-AR, Ty21a-eS-M-N, or Ty21a-eS-M-N-AR was added to the wells in the presence of recombinant Fc tagged SARS-CoV-2 spike RBD protein. Unbound RBD was removed with washing. HRP-conjugated anti-mouse IgG was then applied to the wells in the presence of 3,3′,5,5′-tetramethylbenzidine (TMB) substrate. The HRP-conjugated IgG bound to Fc tagged RBD protein and reacted with the TMB solution, producing a blue color that was proportional to the amount of bound RBD.

### IV.D. Example 4: Foam Drying of Vaccine

Foam drying vaccine. Recombineered Ty21a-eS-AR strain was grown in CY medium supplemented with 0.02% galactose to early stationary phase (Sim, B. K. L., et al., NPJ Vaccines, 2:17 (2017); Ohtake, S., et al., Vaccine 29(15):2761-71 (2011)). The organisms were harvested, resuspended in CY medium, and mixed 1:1 with a 2× foam-drying protective agent (FPA) cocktail in CY medium to achieve a final concentration of 25% trehalose (w/w), 5% fish gelatin (w/w), 0.5% methionine (w/w), and 0.34% potassium phosphate, pH 8.0. Five mL aliquots of the recombineered Ty21a formulation were dispensed into 100-mL Schott vials (Allergy Laboratories, Oklahoma City, Okla.). Foam drying was performed using a Virtis Advantage XL-70 lyophilizer with the following program: Decreasing pressure from 760 Torr to 80 Torr over 15 min at 10° C., holding at 80 Torr for 45 min, decreasing pressure to 5 Torr over 20 min, and a further decrease to 1 Torr at 22° C. with holding for 2 h and further reduction in pressure to <100 m Torr and holding at 15° C. for 48 h. Stoppering was performed in the lyophilizer after argon purging. Residual water content was determined using the Karl Fischer method on a Mettler-Toledo Stromboli balance.

### IV.E. Example 5: Assessment of Stability Profile of Foam Dried Vaccines

Establishing the stability profile of Ty21a-eS-AR using a foam drying process. Stability of the foam dried products (e.g., stability at room temperature (20-25° C.)) is being assessed by residual moisture content and viability (most recent time point at 4 months) (Sim, B. K. L., et al., NPJ Vaccines, 2:17 (2017)). Viability was measured by removing a sample of the foam dried product at one or more time points over a period of time, reconstituting the dried material in an equivalent volume of sterile water to the original pre-foam dried state (ml/mg), and quantitating CFU. CFU were quantitated by plating the reconstituted sample on tryptic soy agar and scoring for CFU/mg in the foam dried product after overnight incubation at 37° C. (Sim, B. K. L., et al., NPJ Vaccines 2:17 (2017)).

### IV.F. Example 6: Immunogenicity of Foam Dried Ty21a-eS and Ty21a-eS-AR Vaccines

Immunogenicity of foam dried vaccine candidates. Mice, hamsters, and/or NHPs (rhesus macaques) are immunized and assessed for: 1) antibody levels, 2) neutralization activity of antibodies, 3) systems serology and functional profiling (NHPs only), 4) antibody dependent enhancement, 5) cellular immune responses (mice and NHPs), 6) VE (hamsters and NHPs).

Immunogenicity in mice (Table 1). S. typhi (Ty21a) can only infect humans (Spanò, S., Adv Exp Med Biol. 915:283-94 (2016)). Thus, the true evaluation of the immunogenicity of Ty21a-eS-AR by the oral route can only be assessed in humans. Nevertheless, with these considerations, is important to assess the humoral and cellular responses induced by immunization to demonstrate that the protein produced by Ty21a-es-AR induces neutralizing antibodies and relevant cellular immune responses. BALB/c mice are immunized by intraperitoneal (IP) injection on days 0, 14 and 28 with 5×10⁷ CFU of Ty21a-eS-AR. The control is Ty21a. (N=10 mice/group). This dose of recombinant Ty21a stimulates robust immune responses (Wu, Y., et al., J Infect Dis. 215:259-68 (2017); Sim, B. K. L., et al., NPJ Vaccines 2:17 (2017)).

TABLE 1
Mice receive 3 doses at 2-week intervals. Serum and
PBMCs collected pre-immunization and 10 days post
dose 3. Splenocytes collected 10 days post dose 3.

| Group | N (M/F) | Immunogen | Volume | # doses | Dose (CFU) |
|---|---|---|---|---|---|
| 1 | 10 (5/5) | Ty21a-eS-AR | 0.2 mL | 3 | 5 × 10⁷ CFU |
| 2 | 10 (5/5) | Ty21a-eS | 0.2 mL | 3 | 5 × 10⁷ CFU |
| 3 | 10 (5/5) | Ty21a | 0.2 mL | 3 | 5 × 10⁷ CFU |

ELISA. Spike proteins for ELISA assessments are purchased (RayBiotech, GA, Sino Biological, and/or provided by Dr. Galit Alter of Ragon Institute). Subclass IgG assessments determine Th1 (IgG2a, IgG3) and Th2 (IgG1) responses. 96-well plates are coated with 1 μg/ml SARS-CoV-2 Spike (S) protein and serial dilutions of serum diluted in casein block are added with standard blocking and washing steps in between (Smith, T. R. F., et al., Nat Commun. 11:2601 (2020)). IgA levels are also measured using recombinant S protein as capture antigen and secondary goat anti-mouse biotin labeled IgA, cat #abx134852 (Abbexa, Houston, Tex.). ELISA antibody levels are defined as the serum dilution at which the optical density (OD) is 0.5 (OD 0.5) (Epstein, J. E., et al., Science 334: 475-80 (2011)).

Neutralization assay. An authentic SARS CoV-2 plaque reduction neutralization assay is used to quantify neutralization, as described (Zhang, Q., et al., Nano Letters 20:5570-4 (2020)). Briefly, a known amount of virus is incubated for 60 minutes with various dilutions of serum from immunized animals. Following incubation, the viable virus is quantified by plaque assay. Dilutions are performed in triplicate and each of the dilutions is assayed in triplicate.

Antibody-dependent enhancement (ADE) studies. The ADE assay are done in HL-CZ, promonocyte or Raji cell lines that are susceptible to SARS-CoV as described previously (Wang, S. F., et al., Biochem Biophys Res Commun. 451:208-14 (2014)). Cells are infected with pseudotyped viruses in the presence of control or test sera at 10- to 2000-fold dilutions and incubated at 37° C. for 48 h. Supernatants and cell lysates are collected and viral titers determined by quantitative RT-PCR or luminescence analyses to determine if test sera increases the viral load in cells. Cell apoptosis is determined by Annexin V staining as described previously (Wang, S. F., et al., Biochem Biophys Res Commun. 451:208-14 (2014)).

Cellular responses. Mice are sacrificed 10 days after the 3^{rd} dose and splenocytes acquired. ELISpot assays are performed to assess IFN-γ and IL-4 responses. 2×10⁵ fresh splenocytes/well are stimulated for 20 h with pools of 15 amino acid peptides overlapping by 9 amino acids and representing the eS (or RBD) as described and provided by Dr. Alex Sette (Smith, T. R. F., et al., Nat Commun. 11:2601 (2020)). ELISpot kits (Mabtech) are used per manufacturer's protocols.

### IV.G. Example 7: Immunogenicity and Vaccine Efficacy of Ty21a-eS-AR

Immunogenicity and vaccine efficacy (VE) in the Syrian hamster model (Chan, J., et al., Clin Infect Dis. 71(9):2428-46 (2020)). The dosing regimen follows that in humans for Ty21a (administered orally) (Vivotif [package insert-USA]. Crucell Switzerland LTD; September 2013. http://www.fda.gov/downloads/BiologicsBloodVaccines/Vaccines/ApprovedProducts/U CM142807.pdf). Hamsters and NHPs are immunized by the intranasal (IN) route, instead of orally, because the oral route for S. typhi/Ty21a is only reliable in humans. Groups of 10 (M=F), 6-10-week-old hamsters are dosed with 5×10⁷ CFU IN on days 1, 3, and 5 (Table 2) at BioQual Inc (https://www.bioqual.com). Sera are collected prior to immunizations and 28 days after the 3^{rd} immunization (day 33, just before challenge) and antibody assays performed as above. Challenge strain SARS-CoV-2 USA-WA1/2020 stock from BEI Resource (NR-52281; Lot #70033175) is used. Virus inoculum is prepared per Bioqual SOPs by serial dilution in PBS as required to reach the intended dose level of 10⁵ pfu in 0.1 mL. Administration of virus is conducted where 0.05 mL of the viral inoculum will be administered dropwise into each nostril, 0.1 mL per animal 4 weeks after the 3^{rd} dose (Chan, J., et al., Clin Infect Dis. 71(9):2428-46 (2020)). See below for VE assessments. Antibody assays is done as in SA1.5.1, but with reagents appropriate for hamsters (e.g. for ELISA, My Biosource.com, San Diego, Calif., Cat #MBS053809). Cellular immunology assays are not done.

TABLE 2
Syrian hamsters are immunized 3 times at 2-day intervals with Ty21a-es-AR or Ty21a
by IN instillation of 0.05 mL per naris on days 1, 3, and 5, and challenged by
IN inoculation of 0.05 mL per naris of SARS-CoV-2 28 days later on day 33.

| | | | | | Total/Dose | Total Challenge |
|---|---|---|---|---|---|---|
| Group | N (M/F) | Immunogen | Volume | # Doses | (CFU) | Dose (PFU) |
| 1 | 10 (5/5) | Ty21a-eS-AR | 0.1 mL | 3 | 5 × 10⁷ | 10⁵ |
| 2 | 10 (5/5) | Ty21a (control) | 0.1 mL | 3 | 5 × 10⁷ | 10⁵ |

Immunogenicity and VE in non-human primates (NHP) Rhesus macaques (Chandrashekar, A., et al., Science 369(6505):812-7 (2020)). Groups of 3, 8-year-old rhesus macaque non-human primates (NHPs) are dosed with 5×10⁸ CFU IN on days 1, 3, 5, and 7 (Table 3) (the regimen in humans for Ty21a taken orally) at BioQual Inc. (https://www.bioqual.com/). Sera and peripheral blood mononuclear cells (PBMCs) are collected prior to first immunization and 14 days (PBMCs) and 28 days (sera, just before challenge) after the 4^{th} immunization, and antibody and T cells assays are done as described below. Challenge strain SARS-CoV-2 USA-WA1/2020 stock from BEI Resource are diluted in PBS to the desired concentration, and the inoculum are back-titrated to verify the dose given. DMEM containing 10⁵ PFU of SARS-CoV-2 in 1 mL are IN inoculated in 0.5 mL per naris as described 4 weeks after the 3^{rd} dose. See below for VE assessments.

TABLE 3
NHPs are immunized 4 times at 2-day intervals with Ty21a-es-AR or Ty21a
by IN instillation of 0.5 mL per naris on days 1, 3, 5, and 7 (the
regimen in humans for Ty21a taken orally) challenged by IN inoculation
of 0.5 mL per naris of SARS-CoV-2 28 days later on day 35.

| | | | | | Total/Dose | Total Challenge |
|---|---|---|---|---|---|---|
| Group | N (M/F) | Immunogen | Volume | # Doses | (CFU) | Dose (PFU) |
| 1 | 4 (2/2) | Ty21a-eS-AR | 1 mL | 4 | 5 × 10⁸ | 10⁵ |
| 2 | 4 (2/2) | Ty21a (control) | 1 mL | 4 | 5 × 10⁸ | 10⁵ |

### IV.H. Example 8: Immunogenicity, Serology, Functional Profiling, and Antibody Dependent Enhancement

Antibody assessments for NHPs. essentially the same assays for ELISA and neutralization assays as described above for mice are used, but rhesus-specific reagents are used as appropriate. Sera taken prior to immunization and prior to challenge are assessed. However, in addition the Alter laboratory at Ragon Institute will conduct the following assessments of systems serology, function, and antibody dependent enhancement (ADE).

Systems Serology. SARS-CoV-2 antigen is carboxy-coupled to Luminex beads. A customized Luminex is used to capture antigen-specific antibody subclass, isotypes, Fc-receptor, lectin-like molecules (DC-Sign, CD23, Dectin), complement initiators (C1q, MBL), innate immune receptors, and lectin binding profiles (SNA: sialic acid, RCA: galactose, AAL: fucose).

Functional profiling. Functional assays covering a range of effector mechanisms (ADCC, phagocytosis, complement, antigen uptake) carried out by a diverse set of innate effector cells (NK, dendritic cells, neutrophils, monocytes, macrophages, etc.) are utilized to assess antibody functionality:

a) Antibody-dependent phagocytosis is assessed using antigen-coated fluorescent bead uptake by monocytes, monocyte-derived mϕs, neutrophils, or monocyte-derived dendritic cells (DCs) (McAndrew, E. G., et al., J Vis Exp. 57:e3588 (2011));

b) Antibody-dependent NK cell degranulation assesses NK cell activation, a correlate of ADCC, following stimulation with antigen-coated bar-coded beads (Jegaskanda, S., et al., J Immunol 190:1837-48 (2013));

c) Antibody-dependent complement deposition assesses deposition of C3b/C3d onto antigen-coated beads following Ab-co-culture in the presence of guinea pig complement;

d) Antibody-dependent cellular cytotoxicity probes Ab-mediated cytotoxicity by innate immune effector cells (NK cells, complement, neutrophils) against antigen-coated NK-resistant CEM cells (Gomez-Roman, V. R., et al., J Immunol Methods 308:53-67 (2006)).

Antibody dependent enhancement (ADE). ADE likely occurs through multiple mechanisms that include suboptimal neutralization by an antibody followed by antibody bound virus infecting a cell via an Fc receptor. In the Ragon Institute BL3, we will employ a cell-based ADE assay to measure uptake and inflammation following antibody-opsonized virus exposure to THP1 cells, similar assays used to evaluate Dengue-virus driven ADE (Chan, K. R., et al., Proc Natl Acad Sci USA 111:2722-7 (2014)). Authentic SARS-CoV-2 virus are co-cultured with virus at multiple MOIs, and then added to the monocyte cell line THP-1 and infection is quantified using anti-SARS CoV-2 antibody staining.

### IV.I. Example 9: Multiparameter Flow Cytometry and Intracellular Cytokine Staining Assessments of NHPs

Multiparameter flow cytometry and in tracellular cytokine staining assessments of NHPs is done as described in previous studies with NHPs with malaria vaccines (Epstein, J. E., et al., Science 334: 475-80 (2011); Ishizuka, A. S., et al., Nat Med. 22:614-23 (2016)), and has been done for COVID-19 vaccines (Chandrashekar, A., et al., Science 369(6505):812-7 (2020)). Focus is on assessing Th1 (e.g. IFN-γ) and Th2 (e.g. IL-4) responses on cryopreserved NHP PBMCs using the same pools of overlapping peptides described in Ishizuka, A. S., et al., Nat Med. 22: 614-23 (2016), and assesses cells from the spleen post-sacrifice. Statistical Analysis of flow cytometry studies are done as we have described (Wang S. F., et al., Biochem Biophys Res Commun. 451:208-14 (2014); Wu, Y, et al., J Infect Dis 215:259-68 (2017)). In brief, data is analyzed using FlowJo v9.8.5 (Tree Star). Statistical analyses are performed with Pestle v1.7 and SPICE v5.3 (M. Roederer) 49, JMP 11 (SAS), Prism 6 (GraphPad), and R v3.2.2 with RStudio v0.99.483. Non-parametric tests are used for association with protection (eg. Kruskal-Wallis with Dunn's post-test correction for multiple comparisons, Wilcoxon matched-pairs signed rank test with Bonferroni correction for multiple comparisons (Ishizuka A. S., et al., Nat Med 22: 614-23 (2016)).

### IV.J. Example 10: Vaccine Efficacy Assessments in Hamsters and NHPs

The following parameters are followed to assess vaccine efficacy in hamsters and NHPs: a) General appearance, weight loss and clinical signs; b) Infectious viral loads and viral RNA in the respiratory tract as determined by testing with oral swabs on days 1, 2, 4 and 7 post challenge in hamsters and NHPs and bronchoalveolar lavage (BAL) of NHPs on days 2, 4, and 7 post-challenge; c) Tissue histopathology, and d) qPCR testing of lungs, nares, GI tract and other tissues after sacrificing the animals.

### IV.K. Example 11: Vaccine Efficacy Assessments of Additional Recombinant Ty21a Strains Expressing SARS-CoV-2 RBD, M, N, eS, and Combinations Thereof

Mice, hamsters, and/or NHPs are immunized with Ty21a-RBD, Ty21a-RBD-AR, Ty21a-M, Ty21a-M-AR, Ty21a-N, Ty21a-N-AR, Ty21a-eS-M-N, Ty21a-eS-M-N-AR, Ty21a-eS+Ty21a-M+Ty21a-N, Ty21a-eS-AR+Ty21a-M-AR+Ty21a-N-AR, or Ty21a and assessed for vaccine efficacy (VE) as described in Examples 6 and 7 with the caveat that M and N proteins and peptide pools will also be used for the antibody and cellular immunology assays.

Mouse immunogenicity. Mice are immunized IP as described above (Table 1). The immunological responses in the 10 vaccine candidates and Ty21a control (Table 4) are used to determine which candidates are assessed in hamsters. If AR and non-AR immunogens have similar immunological responses, the AR immunogen is selected. If the Ty21a-eS and Ty21a-RBD immunogens have similar neutralization titers, Ty21a-eS is selected.

TABLE 4
11 groups of 10 mice (5M/5F) (total = 110) receive 3
doses at 2-week intervals. Serum/PBMCs collected pre-immunization
and 10 days post dose 3. Splenocytes collected 10 days post dose 3.

| Group | Immunogen | Volume | Dose (CFU) |
|---|---|---|---|
| 1 | Ty21a-RBD | 0.2 mL | 5 × 10⁷ |
| 2 | Ty21a-RBD-AR | 0.2 mL | 5 × 10⁷ |
| 3 | Ty21a-M | 0.2 mL | 5 × 10⁷ |
| 4 | Ty21a-M-AR | 0.2 mL | 5 × 10⁷ |
| 5 | Ty21a-N | 0.2 mL | 5 × 10⁷ |
| 6 | Ty21a-N-AR | 0.2 mL | 5 × 10⁷ |
| 7 | Ty21a-eS-M-N | 0.2 mL | 5 × 10⁷ |
| 8 | Ty21a-eS-M-N-AR | 0.2 mL | 5 × 10⁷ |
| 9* | Ty21a-eS + | 0.066 mL + 0.066 | 5.1 × 10⁷ |
| | Ty21a-M + | mL + 0.066 mL | |
| | Ty21a-N | | |
| 10* | Ty21a-eSAR + | 0.066 mL + 0.066 | 5.1 × 10⁷ |
| | Ty21a-M-AR + | mL + 0.066 mL | |
| | Ty21a-N-AR | | |
| 11 | Ty21a | 0.2 mL | 5 × 10⁷ |

*1.7 × 10⁷ CFU of each of the 3 immunogens.

Hamster antibody responses and VE studies. Hamsters are immunized as described above in Table 2 and below in Table 5 for the relevant vaccine candidates with Ty21a alone as control. The final down selection of immunogens is based on the results of the mouse immunogenicity studies (Table 4) and the other considerations described above for the assessment of mouse immunogenicity. Antibody responses and VE are assessed as described above in Examples 6-10.

TABLE 5
Syrian hamsters are immunized 3 times at 2-day intervals by IN instillation
of 0.05 mL per naris on days 1, 3, and 5, and challenged by IN inoculation
of 0.05 mL per naris of SARS-CoV-2 28 days later on day 33.

| | | | | Total Immunizing | Challenge |
|---|---|---|---|---|---|
| Group | N (M/F) | Immunogen | Vol (mL) | Dose (CFU) | Dose (PFU) |
| 1 | 10 (5/5) | Ty21a-eS-M-N-AR | 0.1 mL | 5 × 10⁷ | 10⁵ |
| 2 | 10 (5/5) | Ty21a-eS-AR + | 0.0333 + | 5.1 × 10⁷ | 10⁵ |
| | | Ty21a-M-AR + | 0.0333 + | (1.7 × 10⁷ + 1.7 × | |
| | | Ty21a-N-AR | 0.0333 | (Ty21a-eS construct) × 10⁷) | |
| 3 | 10 (5/5) | Ty21a (control) | 0.1 mL | 5 × 10⁷ | 10⁵ |

NHP immunogenicity and VE studies. NHPs are immunized as described in Table 3 and below in Table 6 for the relevant vaccine candidates with Ty21a alone as control. The final down selection of immunogens is based on the results of the mouse and hamster immunogenicity studies (Tables 4 and 5) and hamster VE studies, and the other considerations described above for the assessment of mouse immunogenicity. Antibody and cellular immune responses and VE are assessed as described above in Examples 6-10.

TABLE 6
NHPs are immunized 4 times at 2-day intervals by IN instillation of 0.5 mL per naris
on days 1, 3, 5, and 7 (regimen in humans for Ty21a taken orally) and challenged
by IN inoculation of 0.5 mL per naris of SARS-CoV-2 28 days later on day 35.

| | | | | Total Immunizing | Challenge |
|---|---|---|---|---|---|
| Group | N (M/F) | Immunogen | Vol (mL) | Dose (CFU) | Doe (PFU) |
| 1 | 4 (2/2) | Ty21a-eS-M-N-AR | 1 | 6 × 10⁸ | 10⁵ |
| 2 | 4 (2/2) | Ty21a-eS-AR + | 0.0333 + | 6 × 10⁸ | 10⁵ |
| | | Ty21a-M-AR + | 0.0333 + | (2 × 10⁸ + 2 × 10⁸ + 2 × 10⁸) | |
| | | Ty21a-N-AR | 0.0333 | | |
| 3 | 4 (2/2) | Ty21a (control) | 1 | 6 × 10⁸ | 10⁵ |

### IV.L. Example 12: Chromosomal Integration of eS Gene Fragment

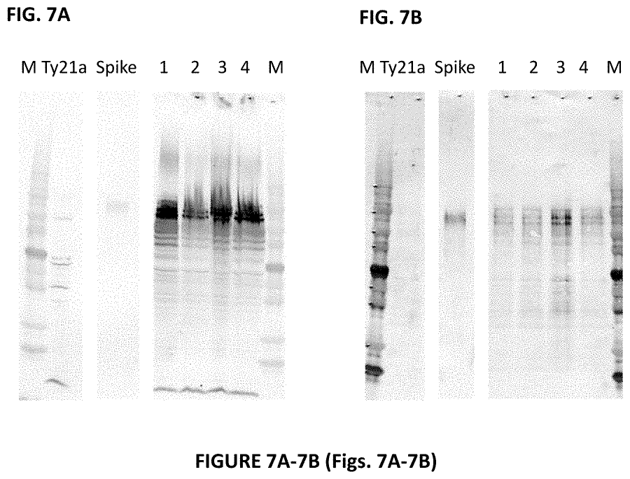

A synthesized sequence of eS fused to the secretion signal Hly (SEQ ID NO: 5; FIG. 9) (Thomas, S., et al., Biochim Biophys Acta. 1843:1629-41 (2014)) was codon optimized for expression in Salmonella and introduced into the shuttle plasmid (pUC57). The eS Hlya cassette was PCR amplified and inserted into plasmid pMDTV-Lpp-PA Hlys after removal of the PA sequence fragment. The resultant plasmid pMDTV-S-ecto was used to transform competent TY21a. The eS fusion protein was strongly expressed in both the pellet (FIG. 7A) as well as in the supernatant (FIG. 7B) of transformed Ty21a containing plasmid Ty21a+pTV-Lpp-S-ecto 9 Hlys (Kan). The results shown in FIGS. 7A-7B demonstrate that Ty21a are capable of efficiently expressing the eS fusion protein.

Next, the gene fragment encoding the eS fusion protein was integrated into the chromosome of Ty21a using k-red recombineering technology (Datsenko K. A., and B. L. Wanner, Proc Natl Acad Sci USA 97:6640-5 (2000); Dharmasena M. N., et al., Int J Med Microbiol. 303:105-13 (2013)) and previously described methods (Wu, Y, et al., J Infect Dis 215:259-68 (2017); Sim, B. K. L., et al., NPJ Vaccines 2:17 (2017)). The constitutive lpp promoter (Lpp-outer membrane lipoprotein) (Inouye S., and M. Inouye, Nucleic Acids Res 13:3101-10 (1985)) was used to ensure high expression of the eS fusion protein from the integrated gene fragment. The gene fragment encoding the eS fusion protein was inserted between tviD and vexA in the chromosome of Ty21a (FIG. 5B), resulting in a Ty21a-S-ecto clone.

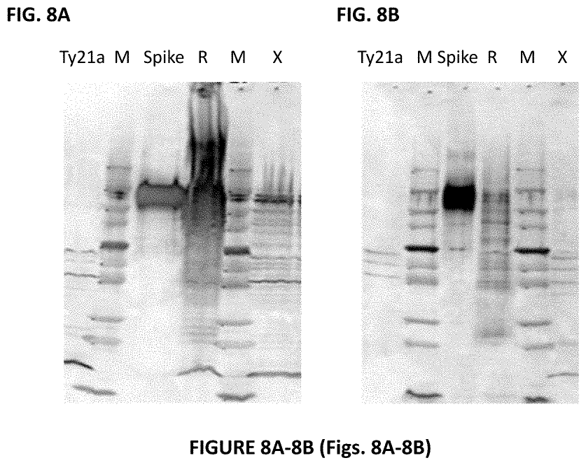

To show that the chromosomally integrated eS gene fragment expressed eS protein in both the pellet and supernatant of the Ty21a-S-ecto clone, a culture of clone Ty21a-S-ecto at OD 0.15 was centrifuged, pelleted, and mixed with sample buffer. Sample buffer was then subjected to electrophoresis on a 4-20% Tris Glycine gel and blotted onto a PVDF membrane. The PVDF membrane was probed with anti-SARS-CoV-2/2019-nCoV Spike polyclonal rabbit antibody (1:1500 dilution) (FIG. 8A). Similarly, supernatant of an OD 0.5 culture of clone Ty21a-S-ecto was mixed with sample buffer and loaded onto a 4-20% Tris Glycine gel, subjected to electrophoresis, blotted onto a PVDF membrane, and probed with anti-SARS-CoV-2/2019-nCoV Spike polyclonal rabbit antibody (1:1500 dilution) (FIG. 8B). The results shown in FIGS. 8A-8B demonstrate that clone Ty21a-S-ecto, which has the eS gene fragment integrated in the chromosome of Ty21a, is capable of efficiently expressing the eS fusion protein

### IV.M. Example 13: Chromosomal Integration of RBD-HlyA

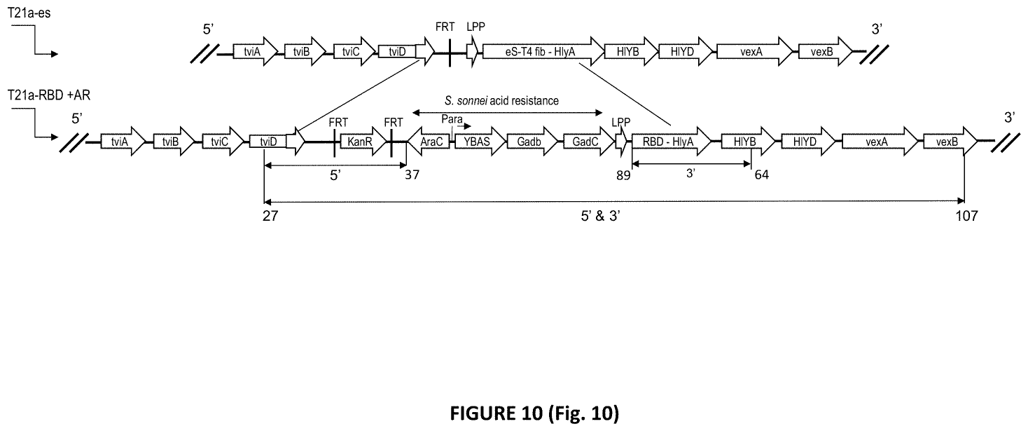

The receptor binding domain (RBD; SEQ ID NO: 11) of the SARS-CoV2 is at position 319 to 541 on the amino acid sequence of the spike protein. The RBD was PCR-amplified from the pUC57 plasmid carrying the entire eS (See Example 1). The RBD was then used to replace the eS gene on the pMDTV-LPP-eS-HlyA+AR plasmid, as follows: The pMDTV-LPP-eS-HlyA+AR plasmid was linearized by PCR, excluding the eS gene. The linearized plasmid without the eS gene was then assembled to the RBD using NEBuilder® HiFi DNA Assembly (New England Bioloabs #E5520S), to generate pMDTV-LPP-RBD-HlyA+AR. The construct was confirmed by PCR and sequencing. The RBD was fused to the HlyA secretion signal (RBD-HlyA). Next, the RBD-HlyA and the Shigella sonnei acid resistance cassette were integrated to the Ty21a genome at the tviE locus as follows: The pMDTV-LPP-RBD-HlyA-AR was used as a template for a PCR, amplifying from the 5′ end of the Tvid to the HlyA. The amplicon (AR-LPP-RBD-HlyA) was used to replace the eS-HlyA in the transgenic Ty21a-eS construct (described in Example 12) by homologous recombination, using the i-red recombineering technology, as was described in Example 1. In this case, the HlyA secretion signal was already present in the transgenic bacteria and was used as the right homologous arm, while the 3′ end of the tviD gene was used as the left homologous arm. The kanamycin resistance gene was used as a selection marker as it was removed in the host Ty21a-eS transgenic strain. A schematic showing the construction of the Ty21a-RBD-AR construct is shown in FIG. 10.

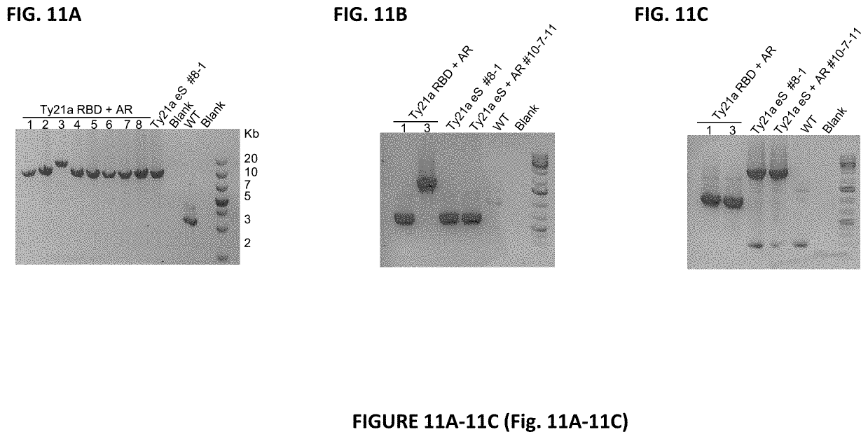

Additionally, PCR reactions were used to confirm full integration of the AR-LPP-RBD-HlyA construct (FIG. 11A-11C). The inserted fragment was PCR-amplified using primer 27 and primer 107, flanking the homologous arms on both 5′ and 3′ ends (FIG. 11A). The expected amplicon sizes were 3.3 Kb for the WT, 9.7 Kb for the background Ty21-eS construct, and 13.3 Kb for the transgenic Ty21-RBD+AR. Out of the 8 clones tested, only clone 3 was positive for the insertion.

Conformation of the integration on the 5′ end was done by PCR using primer 27, upstream to the left homology arm and primer 37, inside the inserted fragment, downstream to the kanR gene (FIG. 11B). The expected amplicon sizes were 0.7 Kb for the background Ty21-eS construct and the Ty21 eS+AR construct, and 2.1 Kb for the transgenic Ty21-RBD+AR. No amplification is expected on WT. Clone 3 was positive for the insertion while clone 1 was not.

Conformation of the integration on the 3′ end was done by PCR using primer 64, downstream to the right homology arm (HlyA), on the HlyB gene and primer 89, inside the inserted fragment, on the 5′ of the RBD (FIG. 11C). The expected amplicon sizes were 4.1 Kb for the background Ty21-eS construct and the Ty21 eS+AR construct, and 1.1 Kb for the transgenic Ty21-RBD+AR. No amplification was expected for WT. Both clones were positive for the insertion on the 3′. The PCR reactions confirmed full integration for clone 3 and only partial, non-complete integration for other clones.

It is to be appreciated that the Detailed Description section, and not the Summary and Abstract sections, is intended to be used to interpret the claims. The Summary and Abstract sections may set forth one or more but not all exemplary aspects or embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects or embodiments will so fully reveal the general nature of the invention that others can, by applying knowledge within the skill of the art, readily modify and/or adapt for various applications such specific aspects or embodiments, without undue experimentation, without departing from the general concept of the present invention. Therefore, such adaptations and modifications are intended to be within the meaning and range of equivalents of the disclosed aspects or embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects or embodiments, but should be defined only in accordance with the following claims and their equivalents.

TABLE 7
Amino Acid and Nucleic Acid Sequences

| | |
|---|---|
| SEQ ID NO: 1 (Modified | MFVFLVLLPLVSSQCVNLTTRTQLPPAYTNSFTRGVYYPDKVFRSSVLHS |
| SARS CoV-2 eS) | TQDLFLPFFSNVTWFHAIHVSGTNGTKRFDNPVLPFNDGVYFASTEKSNI |
| | IRGWIFGTTLDSKTQSLLIVNNATNVVIKVCEFQFCNDPFLGVYYHKNNK |
| | SWMESEFRVYSSANNCTFEYVSQPFLMDLEGKQGNFKNLREFVFKNIDGY |
| | FKIYSKHTPINLVRDLPQGFSALEPLVDLPIGINITRFQTLLALHRSYLT |
| | PGDSSSGWTAGAAAYYVGYLQPRTFLLKYNENGTITDAVDCALDPLSETK |
| | CTLKSFTVEKGIYQTSNFRVQPTESIVRFPNITNLCPFGEVFNATRFASV |
| | YAWNRKRISNCVADYSVLYNSASFSTFKCYGVSPTKLNDLCFTNVYADSF |
| | VIRGDEVRQIAPGQTGKIADYNYKLPDDFTGCVIAWNSNNLDSKVGGNYN |
| | YLYRLFRKSNLKPFERDISTEIYQAGSTPCNGVEGFNCYFPLQSYGFQPT |
| | NGVGYQPYRVVVLSFELLHAPATVCGPKKSTNLVKNKCVNFNFNGLTGTG |
| | VLTESNKKFLPFQQFGRDIADTTDAVRDPQTLEILDITPCSFGGVSVITP |
| | GTNTSNQVAVLYQGVNCTEVPVAIHADQLTPTWRVYSTGSNVFQTRAGCL |
| | IGAEHVNNSYECDIPIGAGICASYQTQTNSPGSASSVASQSIIAYTMSLG |
| | AENSVAYSNNSIAIPTNFTISVTTEILPVSMTKTSVDCTMYICGDSTECS |
| | NLLLQYGSFCTQLNRALTGIAVEQDKNTQEVFAQVKQIYKTPPIKDFGGF |
| | NFSQILPDPSKPSKRSFIEDLLFNKVTLADAGFIKQYGDCLGDIAARDLI |
| | CAQKFNGLTVLPPLLTDEMIAQYTSALLAGTITSGWTFGAGAALQIPFAM |
| | QMAYRFNGIGVTQNVLYENQKLIANQFNSAIGKIQDSLSSTASALGKLQD |
| | VVNQNAQALNTLVKQLSSNFGAISSVLNDILSRLDPPEAEVQIDRLITGR |
| | LQSLQTYVTQQLIRAAEIRASANLAATKMSECVLGQSKRVDFCGKGYHLM |
| | SFPQSAPHGVVFLHVTYVPAQEKNFTTAPAICHDGKAHFPREGVFVSNGT |
| | HWFVTQRNFYEPQIITTDNTFVSGNCDVVIGIVNNTVYDPLQPELDSFKE |
| | ELDKYFKNHTSPDVDLGDISGINASVVNIQKEIDRLNEVAKNLNESLIDL |
| | QELGKYEQYIKWPWYIWLGFIAGLIAIVMVTIMLCCMTSCCSCLKGCCSC |
| | GSCCKFDEDDSEPVLKGVKLHYTGYIPEAPRDGQAYVRKDGEWVLLSTFL |
| SEQ ID NO: 2 (SARS- | MADSNGTITVEELKKLLEQWNLVIGFLFLTWICLLQFAYANRNRFLYIIK |
| CoV2 M Protein) | LIFLWLLWPVTLACFVLAAVYRINWITGGIAIAMACLVGLMWLSYFIASF |
| | RLFARTRSMWSFNPETNILLNVPLHGTILTRPLLESELVIGAVILRGHLR |
| | IAGHHLGRCDIKDLPKEITVATSRTLSYYKLGASQRVAGDSGFAAYSRYR |
| | IGNYKLNTDHSSSSDNIALLVQ |
| SEQ ID NO: 3 (SARS- | MSDNGPQNQRNAPRITFGGPSDSTGSNQNGERSGARSKQRRPQGLPNNTA |
| CoV2 N Protein) | SWFTALTQHGKEDLKFPRGQGVPINTNSSPDDQIGYYRRATRRIRGGDGK |
| | MKDLSPRWYFYYLGTGPEAGLPYGANKDGIIWVATEGALNTPKDHIGTRN |
| | PANNAAIVLQLPQGTTLPKGFYAEGSRGGSQASSRSSSRSRNSSRNSTPG |
| | SSRGTSPARMAGNGGDAALALLLLDRLNQLESKMSGKGQQQQGQTVTKKS |
| | AAEASKKPRQKRTATKAYNVTQAFGRRGPEQTQGNFGDQELIRQGTDYKH |
| | WPQIAQFAPSASAFFGMSRIGMEVTPSGTWLTYTGAIKLDDKDPNFKDQV |
| | ILLNKHIDAYKTFPPTEPKKDKKKKADETQALPQRQKKQQTVTLLPAADL |
| | DDFSKQLQQSMSSADSTQA |
| SEQ ID NO: 4 (Amino | MFVFLVLLPLVSSQCVNLTTRTQLPPAYTNSFTRGVYYPDKVFRSSVLHS |
| Acid Sequence at | TQDLFLPFFSNVTWFHAIHVSGTNGTKRFDNPVLPFNDGVYFASTEKSNI |
| Positions 1-1296 of | IRGWIFGTTLDSKTQSLLIVNNATNVVIKVCEFQFCNDPFLGVYYHKNNK |
| SARS CoV-2 S Protein) | SWMESEFRVYSSANNCTFEYVSQPFLMDLEGKQGNFKNLREFVFKNIDGY |
| | FKIYSKHTPINLVRDLPQGFSALEPLVDLPIGINITRFQTLLALHRSYLT |
| | PGDSSSGWTAGAAAYYVGYLQPRTFLLKYNENGTITDAVDCALDPLSETK |
| | CTLKSFTVEKGIYQTSNFRVQPTESIVRFPNITNLCPFGEVFNATRFASV |
| | YAWNRKRISNCVADYSVLYNSASFSTFKCYGVSPTKLNDLCFTNVYADSF |
| | VIRGDEVRQIAPGQTGKIADYNYKLPDDFTGCVIAWNSNNLDSKVGGNYN |
| | YLYRLFRKSNLKPFERDISTEIYQAGSTPCNGVEGFNCYFPLQSYGFQPT |
| | NGVGYQPYRVVVLSFELLHAPATVCGPKKSTNLVKNKCVNFNFNGLTGTG |
| | VLTESNKKFLPFQQFGRDIADTTDAVRDPQTLEILDITPCSFGGVSVITP |
| | GTNTSNQVAVLYQDVNCTEVPVAIHADQLTPTWRVYSTGSNVFQTRAGCL |
| | IGAEHVNNSYECDIPIGAGICASYQTQTNSPRRARSVASQSIIAYTMSLG |
| | AENSVAYSNNSIAIPTNFTI |
| SEQ ID NO: 5 (Modified | MFVFLVLLPLVSSQCVNLTTRTQLPPAYTNSFTRGVYYPDKVFRSSVLHS |
| SARS CoV-2 eS) | TQDLFLPFFSNVTWFHAIHVSGTNGTKRFDNPVLPFNDGVYFASTEKSNI |
| | IRGWIFGTTLDSKTQSLLIVNNATNVVIKVCEFQFCNDPFLGVYYHKNNK |
| | SWMESEFRVYSSANNCTFEYVSQPFLMDLEGKQGNFKNLREFVFKNIDGY |
| | FKIYSKHTPINLVRDLPQGFSALEPLVDLPIGINITRFQTLLALHRSYLT |
| | PGDSSSGWTAGAAAYYVGYLQPRTFLLKYNENGTITDAVDCALDPLSETK |
| | CTLKSFTVEKGIYQTSNFRVQPTESIVRFPNITNLCPFGEVFNATRFASV |
| | YAWNRKRISNCVADYSVLYNSASFSTFKCYGVSPTKLNDLCFTNVYADSF |
| | VIRGDEVRQIAPGQTGKIADYNYKLPDDFTGCVIAWNSNNLDSKVGGNYN |
| | YLYRLFRKSNLKPFERDISTEIYQAGSTPCNGVEGFNCYFPLQSYGFQPT |
| | NGVGYQPYRVVVLSFELLHAPATVCGPKKSTNLVKNKCVNFNFNGLTGTG |
| | VLTESNKKFLPFQQFGRDIADTTDAVRDPQTLEILDITPCSFGGVSVITP |
| | GTNTSNQVAVLYQGVNCTEVPVAIHADQLTPTWRVYSTGSNVFQTRAGCL |
| | IGAEHVNNSYECDIPIGAGICASYQTQTNSPGSASSVASQSIIAYTMSLG |
| | AENSVAYSNNSIAIPTNFTISVTTEILPVSMTKTSVDCTMYICGDSTECS |
| | NLLLQYGSFCTQLNRALTGIAVEQDKNTQEVFAQVKQIYKTPPIKDFGGF |
| | NFSQILPDPSKPSKRSFIEDLLFNKVTLADAGFIKQYGDCLGDIAARDLI |
| | CAQKFNGLTVLPPLLTDEMIAQYTSALLAGTITSGWTFGAGAALQIPFAM |
| | QMAYRFNGIGVTQNVLYENQKLIANQFNSAIGKIQDSLSSTASALGKLQD |
| | VVNQNAQALNTLVKQLSSNFGAISSVLNDILSRLDPPEAEVQIDRLITGR |
| | LQSLQTYVTQQLIRAAEIRASANLAATKMSECVLGQSKRVDFCGKGYHLM |
| | SFPQSAPHGVVFLHVTYVPAQEKNFTTAPAICHDGKAHFPREGVFVSNGT |
| | HWFVTQRNFYEPQIITTDNTFVSGNCDVVIGIVNNTVYDPLQPELDSFKE |
| | ELDKYFKNHTSPDVDLGDISGINASVVNIQKEIDRLNEVAKNLNESLIDL |
| | QELGKYEQGYIPEAPRDGQAYVRKDGEWVLLSTFL |
| | ALAYGSQGNLNPLINEISKIISAAGNFDVKEERAAASLLQLSGNASDFSY |
| | GRNSITLTASA |
| SEQ ID NO: 6(Ty21a-eS | cccgttcctcattgatttgattgctaacgtcatgagcattactctatcat |
| Construct) | tccagaatgcctcaatgaacaagttgtttgagaaagagtgtcgcaatgtt |
| | gcaaccagagcccttaaatatgtacgccagaagaaaactgaagggcgtct |
| | ggatgaagcattgtctgtattgattagcctgaaacgaattgagcctgatg |
| | tttctcgtctgatgcgtgaatataagcaaattatcagattatttaatgag |
| | tcacggaaggatggcggtagcactatcacgtcttatgaacatctagacta |
| | tgcgaaaaaattactcgtttttgatagcgaaaatgcctatgccttgaaat |
| | atgccgcattaaatgcaatgcatttacgcgactacacgcaggctttgcag |
| | tattggcagcgactggagaaagtgaatggaccaacggagccggtgacaag |
| | gcagatctcgacctgcataaccgcattacaaaaaaatacatcagggaagt |
| | cgtaacccggggtgtaggctggagctgcttcgaagttcctatactttcta |
| | gagaataggaacttcggaataggaacttcaagatcccctcacgctgccgc |
| | aagcactcagggcgcaagggctgctaaaggaagcggaacacgtagaaagc |
| | cagtccgcagaaacggtgctgaccccggatgaatgtcagctactgggcta |
| | tctggacaagggaaaacgcaagcgcaaagagaaagcaggtagcttgcagt |
| | gggcttacatggcgatagctagactgggcggttttatggacagcaagcga |
| | accggaattgccagctggggcgccctctggtaaggttgggaagccctgca |
| | aagtaaactggatggctttcttgccgccaaggatctgatggcgcagggga |
| | tcaagatctgatcaagagacaggatgaggatcgtttcgcatgattgaaca |
| | agatggattgcacgcaggttctccggccgcttgggtggagaggctattcg |
| | gctatgactgggcacaacagacaatcggctgctctgatgccgccgtgttc |
| | cggctgtcagcgcaggggcgcccggttctttttgtcaagaccgacctgtc |
| | cggtgccctgaatgaactgcaggacgaggcagcgcggctatcgtggctgg |
| | ccacgacgggcgttccttgcgcagctgtgctcgacgttgtcactgaagcg |
| | ggaagggactggctgctattgggcgaagtgccggggcaggatctcctgtc |
| | atctcaccttgctcctgccgagaaagtatccatcatggctgatgcaatgc |
| | ggcggctgcatacgcttgatccggctacctgcccattcgaccaccaagcg |
| | aaacatcgcatcgagcgagcacgtactcggatggaagccggtcttgtcga |
| | tcaggatgatctggacgaagagcatcaggggctcgcgccagccgaactgt |
| | tcgccaggctcaaggcgcgcatgcccgacggcgaggatctcgtcgtgacc |
| | catggcgatgcctgcttgccgaatatcatggtggaaaatggccgcttttc |
| | tggattcatcgactgtggccggctgggtgtggcggaccgctatcaggaca |
| | tagcgttggctacccgtgatattgctgaagagcttggcggcgaatgggct |
| | gaccgcttcctcgtgctttacggtatcgccgctcccgattcgcagcgcat |
| | cgccttctatcgccttcttgacgagttcttctgagcgggactctggggtt |
| | cgaaatgaccgaccaagcgacgcccaacctgccatcacgagatttcgatt |
| | ccaccgccgccttctatgaaaggttgggcttcggaatcgttttccgggac |
| | gccggctggatgatcctccagcgcggggatctcatgctggagttcttcgc |
| | ccaccccagcttcaaaagcgctctgaagttcctatactttctagagaata |
| | ggaacttcggaataggaactaaggaggatattcatatatgcattaatgtc |
| | taacaattcgttcaagccgacgccgcttcgcggcgcggcttaactcaagc |
| | gttagatgcactaagcacataattgctcacagccaaactatcaggtcaag |
| | tctgcttttattatttttaagcgtgcataataagccctacacaaattggg |
| | agatatatcatgaaaggctggctttttcttgttatcgcaatagttggcga |
| | agtaatcgcaacatccgcattaaaatctagcgagggctttactgtcgacc |
| | aaaaaaatattgacaacataaaaaactttgtgttatacttgtaacgGact |
| | ctagagggtattaataatgaaaggagaggacatGAGCTCATGTTTGTGTT |
| | TCTGGTGCTGCTGCCGCTGGTGAGCAGCCAGTGCGTGAACCTGACCACCC |
| | GCACCCAGCTGCCGCCGGCGTATACCAACAGCTTTACCCGCGGCGTGTAT |
| | TATCCGGATAAAGTGTTTCGCAGCAGCGTGCTGCATAGCACCCAGGATCT |
| | GTTTCTGCCGTTTTTTAGCAACGTGACCTGGTTTCATGCGATTCATGTGA |
| | GCGGCACCAACGGCACCAAACGCTTTGATAACCCGGTGCTGCCGTTTAAC |
| | GATGGCGTGTATTTTGCGAGCACCGAAAAAAGCAACATTATTCGCGGCTG |
| | GATTTTTGGCACCACCCTGGATAGCAAAACCCAGAGCCTGCTGATTGTGA |
| | ACAACGCGACCAACGTGGTGATTAAAGTGTGCGAATTTCAGTTTTGCAAC |
| | GATCCGTTTCTGGGCGTGTATTATCATAAAAACAACAAAAGCTGGATGGA |
| | AAGCGAATTTCGCGTGTATAGCAGCGCGAACAACTGCACCTTTGAATATG |
| | TGAGCCAGCCGTTTCTGATGGATCTGGAAGGCAAACAGGGCAACTTTAAA |
| | AACCTGCGCGAATTTGTGTTTAAAAACATTGATGGCTATTTTAAAATTTA |
| | TAGCAAACATACCCCGATTAACCTGGTGCGCGATCTGCCGCAGGGCTTTA |
| | GCGCGCTGGAACCGCTGGTGGATCTGCCGATTGGCATTAACATTACCCGC |
| | TTTCAGACCCTGCTGGCGCTGCATCGCAGCTATCTGACCCCGGGCGATAG |
| | CAGCAGCGGCTGGACCGCGGGCGCGGCGGCGTATTATGTGGGCTATCTGC |
| | AGCCGCGCACCTTTCTGCTGAAATATAACGAAAACGGCACCATTACCGAT |
| | GCGGTGGATTGCGCGCTGGATCCGCTGAGCGAAACCAAATGCACCCTGAA |
| | AAGCTTTACCGTGGAAAAAGGCATTTATCAGACCAGCAACTTTCGCGTGC |
| | AGCCGACCGAAAGCATTGTGCGCTTTCCGAACATTACCAACCTGTGCCCG |
| | TTTGGCGAAGTGTTTAACGCGACCCGCTTTGCGAGCGTGTATGCGTGGAA |
| | CCGCAAACGCATTAGCAACTGCGTGGCGGATTATAGCGTGCTGTATAACA |
| | GCGCGAGCTTTAGCACCTTTAAATGCTATGGCGTGAGCCCGACCAAACTG |
| | AACGATCTGTGCTTTACCAACGTGTATGCGGATAGCTTTGTGATTCGCGG |
| | CGATGAAGTGCGCCAGATTGCGCCGGGCCAGACCGGCAAAATTGCGGATT |
| | ATAACTATAAACTGCCGGATGATTTTACCGGCTGCGTGATTGCGTGGAAC |
| | AGCAACAACCTGGATAGCAAAGTGGGCGGCAACTATAACTATCTGTATCG |
| | CCTGTTTCGCAAAAGCAACCTGAAACCGTTTGAACGCGATATTAGCACCG |
| | AAATTTATCAGGCGGGCAGCACCCCGTGCAACGGCGTGGAAGGCTTTAAC |
| | TGCTATTTTCCGCTGCAGAGCTATGGCTTTCAGCCGACCAACGGCGTGGG |
| | CTATCAGCCGTATCGCGTGGTGGTGCTGAGCTTTGAACTGCTGCATGCGC |
| | CGGCGACCGTGTGCGGCCCGAAAAAAAGCACCAACCTGGTGAAAAACAAA |
| | TGCGTGAACTTTAACTTTAACGGCCTGACCGGCACCGGCGTGCTGACCGA |
| | AAGCAACAAAAAATTTCTGCCGTTTCAGCAGTTTGGCCGCGATATTGCGG |
| | ATACCACCGATGCGGTGCGCGATCCGCAGACCCTGGAAATTCTGGATATT |
| | ACCCCGTGCAGCTTTGGCGGCGTGAGCGTGATTACCCCGGGCACCAACAC |
| | CAGCAACCAGGTGGCGGTGCTGTATCAGGGCGTGAACTGCACCGAAGTGC |
| | CGGTGGCGATTCATGCGGATCAGCTGACCCCGACCTGGCGCGTGTATAGC |
| | ACCGGCAGCAACGTGTTTCAGACCCGCGCGGGCTGCCTGATTGGCGCGGA |
| | ACATGTGAACAACAGCTATGAATGCGATATTCCGATTGGCGCGGGCATTT |
| | GCGCGAGCTATCAGACCCAGACCAACAGCCCGGGCAGCGCGAGCAGCGTG |
| | GCGAGCCAGAGCATTATTGCGTATACCATGAGCCTGGGCGCGGAAAACAG |
| | CGTGGCGTATAGCAACAACAGCATTGCGATTCCGACCAACTTTACCATTA |
| | GCGTGACCACCGAAATTCTGCCGGTGAGCATGACCAAAACCAGCGTGGAT |
| | TGCACCATGTATATTTGCGGCGATAGCACCGAATGCAGCAACCTGCTGCT |
| | GCAGTATGGCAGCTTTTGCACCCAGCTGAACCGCGCGCTGACCGGCATTG |
| | CGGTGGAACAGGATAAAAACACCCAGGAAGTGTTTGCGCAGGTGAAACAG |
| | ATTTATAAAACCCCGCCGATTAAAGATTTTGGCGGCTTTAACTTTAGCCA |
| | GATTCTGCCGGATCCGAGCAAACCGAGCAAACGCAGCTTTATTGAAGATC |
| | TGCTGTTTAACAAAGTGACCCTGGCGGATGCGGGCTTTATTAAACAGTAT |
| | GGCGATTGCCTGGGCGATATTGCGGCGCGCGATCTGATTTGCGCGCAGAA |
| | ATTTAACGGCCTGACCGTGCTGCCGCCGCTGCTGACCGATGAAATGATTG |
| | CGCAGTATACCAGCGCGCTGCTGGCGGGCACCATTACCAGCGGCTGGACC |
| | TTTGGCGCGGGCGCGGCGCTGCAGATTCCGTTTGCGATGCAGATGGCGTA |
| | TCGCTTTAACGGCATTGGCGTGACCCAGAACGTGCTGTATGAAAACCAGA |
| | AACTGATTGCGAACCAGTTTAACAGCGCGATTGGCAAAATTCAGGATAGC |
| | CTGAGCAGCACCGCGAGCGCGCTGGGCAAACTGCAGGATGTGGTGAACCA |
| | GAACGCGCAGGCGCTGAACACCCTGGTGAAACAGCTGAGCAGCAACTTTG |
| | GCGCGATTAGCAGCGTGCTGAACGATATTCTGAGCCGCCTGGATCCGCCG |
| | GAAGCGGAAGTGCAGATTGATCGCCTGATTACCGGCCGCCTGCAGAGCCT |
| | GCAGACCTATGTGACCCAGCAGCTGATTCGCGCGGCGGAAATTCGCGCGA |
| | GCGCGAACCTGGCGGCGACCAAAATGAGCGAATGCGTGCTGGGCCAGAGC |
| | AAACGCGTGGATTTTTGCGGCAAAGGCTATCATCTGATGAGCTTTCCGCA |
| | GAGCGCGCCGCATGGCGTGGTGTTTCTGCATGTGACCTATGTGCCGGCGC |
| | AGGAAAAAAACTTTACCACCGCGCCGGCGATTTGCCATGATGGCAAAGCG |
| | CATTTTCCGCGCGAAGGCGTGTTTGTGAGCAACGGCACCCATTGGTTTGT |
| | GACCCAGCGCAACTTTTATGAACCGCAGATTATTACCACCGATAACACCT |
| | TTGTGAGCGGCAACTGCGATGTGGTGATTGGCATTGTGAACAACACCGTG |
| | TATGATCCGCTGCAGCCGGAACTGGATAGCTTTAAAGAAGAACTGGATAA |
| | ATATTTTAAAAACCATACCAGCCCGGATGTGGATCTGGGCGATATTAGCG |
| | GCATTAACGCGAGCGTGGTGAACATTCAGAAAGAAATTGATCGCCTGAAC |
| | GAAGTGGCGAAAAACCTGAACGAAAGCCTGATTGATCTGCAGGAACTGGG |
| | CAAATATGAACAGGGCTATATTCCGGAAGCGCCGCGCGATGGCCAGGCGT |
| | ATGTGCGCAAAGATGGCGAATGGGTGCTGCTGAGCACCTTTCTGGCATTA |
| | GCCTATGGAAGTCAGGGTAATCTTAATCCATTAATTAATGAAATCAGCAA |
| | AATCATTTCAGCTGCAGGTAATTTTGATGTTAAAGAGGAAAGAGCGGCCG |
| | CGTCTTTATTGCAGTTGTCCGGTAATGCCAGTGATTTTTCATATGGACGG |
| | AACTCAATAACTTTGACAGCATCAGCATAAGAGCTCtttattaatttaaa |
| | taatagcaatcttactgggctgtgccacataagattgctatttttttgga |
| | gtcataatggattcttgtcataaaattgattatgggttatacgccctgga |
| | gattttagcccaataccataacgtctctgttaacccggaagaaattaaac |
| | atagatttgacacagacgggactggtctgggattaacgtcatggttgctt |
| | gctgcgaaatctttagaactaaaggtaaaacaggtaaaaaaaacaattga |
| | ccgattaaactttatttctctgcccgcattagtctggagagaggatggat |
| | gtcattttattctgactaaagtcagtaaagaagcaaacagatatcttatt |
| | tttgatctggagcagcgaaatccccgtgttctcgaacagtctgagtttga |
| | ggcgttatatcaggggcatattattcttattgcttcccgttcttctgtta |
| | ccgggaaactggcaaaatttgactttacctggtttattcctgccattata |
| | aaatacaggaaaatatttattgaaacccttgttgtatctgtttttttaca |
| | attatttgcattaataaccccccttttttttcaggtggttatggacaaag |
| | tattagtacacagggggttttcaacccttaatgttattactgtcgcatta |
| | tctgttgtggtggtgtttgagattatactcagcggtttaagaacttacat |
| | ttttgcacatagtacaagtcggattgatgttgagttgggtgccaaactct |
| | tccggcatttactggcgctaccgatctcttattttgagagtcgtcgtgtt |
| | ggtgatactgttgccagggtaagagaattagaccagatccgtaatttcct |
| | gacaggacaggcattaacatctgttctggacttattattttcattcatat |
| | tttttgcggtaatgtggtattacagcccaaagcttactctggtgatctta |
| | ttttcgctgccctgttatgctgcatggtctgtttttattagccccatttt |
| | gcgacgtcgccttgatgataagttttcacggaatgcggataatcaatctt |
| | tcctggtggaatcagtcacggcgattaacactataaaagctatggcagtc |
| | tcacctcagatgacgaacatatgggacaaacaattggcaggatatgttgc |
| | tgcaggctttaaagtgacagtattagccaccattggtcaacaaggaatac |
| | agttaatacaaaagactgttatgatcatcaacctgtggttgggtgcacac |
| | ctggttatttccggggatttaagtattggtcagttaattgcttttaatat |
| | gcttgcaggtcagattgttgcaccggttattcgccttgcacaaatctggc |
| | aggatttccagcaggttggtatatcagttacccgccttggtgatgtgctt |
| | aactctccaactgaaagttatcatgggaaactggcattaccggaaattaa |
| | tggtgatatcacttttcgtaatatccggtttcgctataagcctgactctc |
| | cggttattttagataatatcaatctcagtattaagcagggggaggttatt |
| | ggtattgtcggacgttctggttcaggaaaaagcacattaactaaattaat |
| | tcaacgtttttatattcctgaaaatggccaggtcttaattgatggacatg |
| | atcttgcgttggccgatcctaactggttacgtcgtcaggtgggggttgtg |
| | ttgcaggacaatgtgctgcttaatcgcagtattattgataatatttcact |
| | ggctaatcctggcatgtccgtcgaaaaagttatttatgcagcgaaattag |
| | caggtgctcatgattttatttctgaattgcgtgaggggtataacaccatt |
| | gtcggggaacagggggcaggattatccggaggtcaacgtcaacgcatcgc |
| | aattgcaagggcgctggtgaacaaccctaaaatactcatctttgatgaag |
| | caaccagtgctctggattatgagtcggagcatgtcatcatgcgcaatatg |
| | cacaaaatatgtaagggcagaacggttataatcattgctcatcgtctgtc |
| | tacagtaaaaaatgcagaccgcattattgtcatggaaaaagggaaaattg |
| | ttgaacagggtaaacataaggagctgctttctgaaccggaaagtttatac |
| | agttacttatatcagttacagtcagactaacagaaagaacagaagaatat |
| | gaaaacatggttaatggggttcagcgagttcctgttgcgctataaacttg |
| | tctggagtgaaacatggaaaatccggaagcagttagatactccggtacgt |
| | gaaaaggacgaaaatgaattcttacccgctcatctggaattaattgaaac |
| | gccggtatccagacggccgcgtctggttgcttattttattatggggtttc |
| | tggttattgctttcattttatctgttttaggccaggtggaaattgttgcc |
| | actgcaaatgggaaattaacactcagtgggcgtagcaaagaaattaaacc |
| | tattgaaaactcgatagttaaagaaattatcgtaaaagaaggagagtcag |
| | tccggaaaggggatgtgttattaaagcttacagcgctgggagctgaagct |
| | gatacgttaaaaacgcagtcatcactgttacaggccaggctggaacaaat |
| | tcggtatcaaattctgagccggtcaattgaattaaataaacttcctgaac |
| | tgaaacttcctgatgagccttattttcagaatgtatctgaagaggaagta |
| | ctgcgtttaacttctttgataaaagaacagttttccacatggcaaaatca |
| | gaagtatcaaaaagaactgaatctggataagaaaagagcagagcgattaa |
| | caatacttgcccgtataaaccgttatgaaaatgtatcgagggttgaaaaa |
| | agccgtctggatgatttcaggagcctgttgcataaacaggcaattgcaaa |
| | acatgctgtacttgagcaggagaataaatatgttgaggcagcaaatgaat |
| | tacgggtttataaatcgcaactggagcaaattgagagtgagatattgtct |
| | gcaaaagaagaatatcagcttgtcacgcagctttttaaaaatgaaatttt |
| | agacaagctaagacaaacaacagacagcattgagttattaactctggagt |
| | tagagaaaaatgaagagcgtcaacaggcttcagtaatcagggcccctgtt |
| | tcgggaaaagttcagcaactgaaggttcatactgaaggtggggttgttac |
| | aacagcggaaacactgatggtcatcgttccggaagatgacacgctggagg |
| | ttactgctctggtacaaaataaagatattggttttattaacgtcgggcag |
| | aatgccatcattaaagtggaggcttttccttacacccgatatggttatct |
| | ggtgggtaaggtaaaaaatataaatttagatgcaatagaagaccagaaac |
| | tgggactcgtttttaatgtcattgtttctgttgaagagaatgatttgtca |
| | accgggaataagcacattccattaagctcgggtatggctgtcactgcaga |
| | aataaagactggaatgcgaagcgtaatcagttatcttcttagtcccctgg |
| | aagaatctgtaacagaaagtttacatgagcgttaagtctcagagcagcgg |
| | tatccggctcatatcttctcctgtcagatccaagggcgaattccagcaca |
| | ctggcggccgttactagtggatccaccggtctcgaggggcgcgccggtac |
| | cgaattctaattaatgggcatcatttttcagctatttcatttataaaata |
| | agttatgaaaaaaatcatcatattactaacgacatttttcctgctttcgg |
| | gatgcactattcccagggcggtatttaaatccagccttattaatcaggac |
| | gatcctcgttataatctggtcgaagtcacgccgacattaaaactaagcgc |
| | tcccgatactgtgccgaaaactattgtcgatccggtttttgccgcaaata |
| | actggcactggacatctttggctaaaggcgatgtgctgcatatcactatt |
| | ttatcctcgggcggggctggatatttatccaataacgcgagcggcgaccg |
| | tgcggattttgaaaatattcttgtgactgacagtaataccgttcaggtgc |
| | cttatgccgggacaatcccggtttctggattggatgtgacgcaactggct |
| | gatgagatcaaaaagcgactttcgcgcgttgtcctgaatcctcaggtgat |
| | tgtgacacttaccgcccgcaccggagccatggtgacggtcgagggcagcg |
| | ggaaaacgggtcgataccctctcgaacagagtatgaatcgtctgagtcat |
| | cttttggcaacggcagtggcagtagaaaataccagcacagatatgatgga |
| | agttcacgtgacgcgccagcagcattatttcactgcgcgactgtcagata |
| | tttatcagtatcccggattggatattgccttacagccggatgaccgtatc |
| | actctgcgtcaagtgaccgagtatgttaatgtgctcggcgcagcaggtgt |
| | tcaggggaaacatgctctggttcagcgtcattccagcgtcgtggatgctt |
| | tggcgctcgcgaaaggattaaatgacaatcttgccgatccgcaagcgatt |
| | tttctgtacaagcataacgaagcggaacaggcgaaacagcagatgcgtaa |
| | gctgaatatctatcatgtcgatatgagccaacctaactcggtgtttttag |
| | cgcaggccatacgagtggacaacggcgatgttatctatatctcgaacgcc |
| | tctttgactgatttcgctaaggtgaaagccgcattcgatagctttttgac |
| | ccgcggcactaattctttctaa |
| SEQ ID NO: 7 (Ty21a- | cccgttcctcattgatttgattgctaacgtcatgagcattactctatcat |
| M-N Construct) | tccagaatgcctcaatgaacaagttgtttgagaaagagtgtcgcaatgtt |
| | gcaaccagagcccttaaatatgtacgccagaagaaaactgaagggcgtct |
| | ggatgaagcattgtctgtattgattagcctgaaacgaattgagcctgatg |
| | tttctcgtctgatgcgtgaatataagcaaattatcagattatttaatgag |
| | tcacggaaggatggcggtagcactatcacgtcttatgaacatctagacta |
| | tgcgaaaaaattactcgtttttgatagcgaaaatgcctatgccttgaaat |
| | atgccgcattaaatgcaatgcatttacgcgactacacgcaggctttgcag |
| | tattggcagcgactggagaaagtgaatggaccaacggagccggtgacaag |
| | gcagatctcgacctgcataaccgcattacaaaaaaatacatcagggaagt |
| | cgtaacccggggtgtaggctggagctgcttcgaagttcctatactttcta |
| | gagaataggaacttcggaataggaacttcaagatcccctcacgctgccgc |
| | aagcactcagggcgcaagggctgctaaaggaagcggaacacgtagaaagc |
| | cagtccgcagaaacggtgctgaccccggatgaatgtcagctactgggcta |
| | tctggacaagggaaaacgcaagcgcaaagagaaagcaggtagcttgcagt |
| | gggcttacatggcgatagctagactgggcggttttatggacagcaagcga |
| | accggaattgccagctggggcgccctctggtaaggttgggaagccctgca |
| | aagtaaactggatggctttcttgccgccaaggatctgatggcgcagggga |
| | tcaagatctgatcaagagacaggatgaggatcgtttcgcatgattgaaca |
| | agatggattgcacgcaggttctccggccgcttgggtggagaggctattcg |
| | gctatgactgggcacaacagacaatcggctgctctgatgccgccgtgttc |
| | cggctgtcagcgcaggggcgcccggttctttttgtcaagaccgacctgtc |
| | cggtgccctgaatgaactgcaggacgaggcagcgcggctatcgtggctgg |
| | ccacgacgggcgttccttgcgcagctgtgctcgacgttgtcactgaagcg |
| | ggaagggactggctgctattgggcgaagtgccggggcaggatctcctgtc |
| | atctcaccttgctcctgccgagaaagtatccatcatggctgatgcaatgc |
| | ggcggctgcatacgcttgatccggctacctgcccattcgaccaccaagcg |
| | aaacatcgcatcgagcgagcacgtactcggatggaagccggtcttgtcga |
| | tcaggatgatctggacgaagagcatcaggggctcgcgccagccgaactgt |
| | tcgccaggctcaaggcgcgcatgcccgacggcgaggatctcgtcgtgacc |
| | catggcgatgcctgcttgccgaatatcatggtggaaaatggccgcttttc |
| | tggattcatcgactgtggccggctgggtgtggcggaccgctatcaggaca |
| | tagcgttggctacccgtgatattgctgaagagcttggcggcgaatgggct |
| | gaccgcttcctcgtgctttacggtatcgccgctcccgattcgcagcgcat |
| | cgccttctatcgccttcttgacgagttcttctgagcgggactctggggtt |
| | cgaaatgaccgaccaagcgacgcccaacctgccatcacgagatttcgatt |
| | ccaccgccgccttctatgaaaggttgggcttcggaatcgttttccgggac |
| | gccggctggatgatcctccagcgcggggatctcatgctggagttcttcgc |
| | ccaccccagcttcaaaagcgctctgaagttcctatactttctagagaata |
| | ggaacttcggaataggaactaaggaggatattcatatatgcattaatgtc |
| | taacaattcgttcaagccgacgccgcttcgcggcgcggcttaactcaagc |
| | gttagatgcactaagcacataattgctcacagccaaactatcaggtcaag |
| | tctgcttttattatttttaagcgtgcataataagccctacacaaattggg |
| | agatatatcatgaaaggctggctttttcttgttatcgcaatagttggcga |
| | agtaatcgcaacatccgcattaaaatctagcgagggctttactgtcgacc |
| | aaaaaaatattgacaacataaaaaactttgtgttatacttgtaacgGact |
| | ctagagggtattaataatgaaaggagaggacatGAGCTCCCTAGGATGAG |
| | CGATAACGGCCCGCAGAACCAGCGCAACGCGCCGCGCATTACCTTTGGCG |
| | GCCCGAGCGATAGCACCGGCAGCAACCAGAACGGCGAACGCAGCGGCGCG |
| | CGCAGCAAACAGCGCCGCCCGCAGGGCCTGCCGAACAACACCGCGAGCTG |
| | GTTTACCGCGCTGACCCAGCATGGCAAAGAAGATCTGAAATTTCCGCGCG |
| | GCCAGGGCGTGCCGATTAACACCAACAGCAGCCCGGATGATCAGATTGGC |
| | TATTATCGCCGCGCGACCCGCCGCATTCGCGGCGGCGATGGCAAAATGAA |
| | AGATCTGAGCCCGCGCTGGTATTTTTATTATCTGGGCACCGGCCCGGAAG |
| | CGGGCCTGCCGTATGGCGCGAACAAAGATGGCATTATTTGGGTGGCGACC |
| | GAAGGCGCGCTGAACACCCCGAAAGATCATATTGGCACCCGCAACCCGGC |
| | GAACAACGCGGCGATTGTGCTGCAGCTGCCGCAGGGCACCACCCTGCCGA |
| | AAGGCTTTTATGCGGAAGGCAGCCGCGGCGGCAGCCAGGCGAGCAGCCGC |
| | AGCAGCAGCCGCAGCCGCAACAGCAGCCGCAACAGCACCCCGGGCAGCAG |
| | CCGCGGCACCAGCCCGGCGCGCATGGCGGGCAACGGCGGCGATGCGGCGC |
| | TGGCGCTGCTGCTGCTGGATCGCCTGAACCAGCTGGAAAGCAAAATGAGC |
| | GGCAAAGGCCAGCAGCAGCAGGGCCAGACCGTGACCAAAAAAAGCGCGGC |
| | GGAAGCGAGCAAAAAACCGCGCCAGAAACGCACCGCGACCAAAGCGTATA |
| | ACGTGACCCAGGCGTTTGGCCGCCGCGGCCCGGAACAGACCCAGGGCAAC |
| | TTTGGCGATCAGGAACTGATTCGCCAGGGCACCGATTATAAACATTGGCC |
| | GCAGATTGCGCAGTTTGCGCCGAGCGCGAGCGCGTTTTTTGGCATGAGCC |
| | GCATTGGCATGGAAGTGACCCCGAGCGGCACCTGGCTGACCTATACCGGC |
| | GCGATTAAACTGGATGATAAAGATCCGAACTTTAAAGATCAGGTGATTCT |
| | GCTGAACAAACATATTGATGCGTATAAAACCTTTCCGCCGACCGAACCGA |
| | AAAAAGATAAAAAAAAAAAAGCGGATGAAACCCAGGCGCTGCCGCAGCGC |
| | CAGAAAAAACAGCAGACCGTGACCCTGCTGCCGGCGGCGGATCTGGATGA |
| | TTTTAGCAAACAGCTGCAGCAGAGCATGAGCAGCGCGGATAGCACCCAGG |
| | CGCCTAGGCTTAAGATGGCGGATAGCAACGGCACCATTACCGTGGAAGAA |
| | CTGAAAAAACTGCTGGAACAGTGGAACCTGGTGATTGGCTTTCTGTTTCT |
| | GACCTGGATTTGCCTGCTGCAGTTTGCGTATGCGAACCGCAACCGCTTTC |
| | TGTATATTATTAAACTGATTTTTCTGTGGCTGCTGTGGCCGGTGACCCTG |
| | GCGTGCTTTGTGCTGGCGGCGGTGTATCGCATTAACTGGATTACCGGCGG |
| | CATTGCGATTGCGATGGCGTGCCTGGTGGGCCTGATGTGGCTGAGCTATT |
| | TTATTGCGAGCTTTCGCCTGTTTGCGCGCACCCGCAGCATGTGGAGCTTT |
| | AACCCGGAAACCAACATTCTGCTGAACGTGCCGCTGCATGGCACCATTCT |
| | GACCCGCCCGCTGCTGGAAAGCGAACTGGTGATTGGCGCGGTGATTCTGC |
| | GCGGCCATCTGCGCATTGCGGGCCATCATCTGGGCCGCTGCGATATTAAA |
| | GATCTGCCGAAAGAAATTACCGTGGCGACCAGCCGCACCCTGAGCTATTA |
| | TAAACTGGGCGCGAGCCAGCGCGTGGCGGGCGATAGCGGCTTTGCGGCGT |
| | ATAGCCGCTATCGCATTGGCAACTATAAACTGAACACCGATCATAGCAGC |
| | AGCAGCGATAACATTGCGCTGCTGGTGCAGCTTAAGGCATTAGCCTATGG |
| | AAGTCAGGGTAATCTTAATCCATTAATTAATGAAATCAGCAAAATCATTT |
| | CAGCTGCAGGTAATTTTGATGTTAAAGAGGAAAGAGCGGCCGCGTCTTTA |
| | TTGCAGTTGTCCGGTAATGCCAGTGATTTTTCATATGGACGGAACTCAAT |
| | AACTTTGACAGCATCAGCATAAGAGCTCtttattaatttaaataatagca |
| | atcttactgggctgtgccacataagattgctatttttttggagtcataat |
| | ggattcttgtcataaaattgattatgggttatacgccctggagattttag |
| | cccaataccataacgtctctgttaacccggaagaaattaaacatagattt |
| | gacacagacgggactggtctgggattaacgtcatggttgcttgctgcgaa |
| | atctttagaactaaaggtaaaacaggtaaaaaaaacaattgaccgattaa |
| | actttatttctctgcccgcattagtctggagagaggatggatgtcatttt |
| | attctgactaaagtcagtaaagaagcaaacagatatcttatttttgatct |
| | ggagcagcgaaatccccgtgttctcgaacagtctgagtttgaggcgttat |
| | atcaggggcatattattcttattgcttcccgttcttctgttaccgggaaa |
| | ctggcaaaatttgactttacctggtttattcctgccattataaaatacag |
| | gaaaatatttattgaaacccttgttgtatctgtttttttacaattatttg |
| | cattaataaccccccttttttttcaggtggttatggacaaagtattagta |
| | cacagggggttttcaacccttaatgttattactgtcgcattatctgttgt |
| | ggtggtgtttgagattatactcagcggtttaagaacttacatttttgcac |
| | atagtacaagtcggattgatgttgagttgggtgccaaactcttccggcat |
| | ttactggcgctaccgatctcttattttgagagtcgtcgtgttggtgatac |
| | tgttgccagggtaagagaattagaccagatccgtaatttcctgacaggac |
| | aggcattaacatctgttctggacttattattttcattcatattttttgcg |
| | gtaatgtggtattacagcccaaagcttactctggtgatcttattttcgct |
| | gccctgttatgctgcatggtctgtttttattagccccattttgcgacgtc |
| | gccttgatgataagttttcacggaatgcggataatcaatctttcctggtg |
| | gaatcagtcacggcgattaacactataaaagctatggcagtctcacctca |
| | gatgacgaacatatgggacaaacaattggcaggatatgttgctgcaggct |
| | ttaaagtgacagtattagccaccattggtcaacaaggaatacagttaata |
| | caaaagactgttatgatcatcaacctgtggttgggtgcacacctggttat |
| | ttccggggatttaagtattggtcagttaattgcttttaatatgcttgcag |
| | gtcagattgttgcaccggttattcgccttgcacaaatctggcaggatttc |
| | cagcaggttggtatatcagttacccgccttggtgatgtgcttaactctcc |
| | aactgaaagttatcatgggaaactggcattaccggaaattaatggtgata |
| | tcacttttcgtaatatccggtttcgctataagcctgactctccggttatt |
| | ttagataatatcaatctcagtattaagcagggggaggttattggtattgt |
| | cggacgttctggttcaggaaaaagcacattaactaaattaattcaacgtt |
| | tttatattcctgaaaatggccaggtcttaattgatggacatgatcttgcg |
| | ttggccgatcctaactggttacgtcgtcaggtgggggttgtgttgcagga |
| | caatgtgctgcttaatcgcagtattattgataatatttcactggctaatc |
| | ctggcatgtccgtcgaaaaagttatttatgcagcgaaattagcaggtgct |
| | catgattttatttctgaattgcgtgaggggtataacaccattgtcgggga |
| | acagggggcaggattatccggaggtcaacgtcaacgcatcgcaattgcaa |
| | gggcgctggtgaacaaccctaaaatactcatctttgatgaagcaaccagt |
| | gctctggattatgagtcggagcatgtcatcatgcgcaatatgcacaaaat |
| | atgtaagggcagaacggttataatcattgctcatcgtctgtctacagtaa |
| | aaaatgcagaccgcattattgtcatggaaaaagggaaaattgttgaacag |
| | ggtaaacataaggagctgctttctgaaccggaaagtttatacagttactt |
| | atatcagttacagtcagactaacagaaagaacagaagaatatgaaaacat |
| | ggttaatggggttcagcgagttcctgttgcgctataaacttgtctggagt |
| | gaaacatggaaaatccggaagcagttagatactccggtacgtgaaaagga |
| | cgaaaatgaattcttacccgctcatctggaattaattgaaacgccggtat |
| | ccagacggccgcgtctggttgcttattttattatggggtttctggttatt |
| | gctttcattttatctgttttaggccaggtggaaattgttgccactgcaaa |
| | tgggaaattaacactcagtgggcgtagcaaagaaattaaacctattgaaa |
| | actcgatagttaaagaaattatcgtaaaagaaggagagtcagtccggaaa |
| | ggggatgtgttattaaagcttacagcgctgggagctgaagctgatacgtt |
| | aaaaacgcagtcatcactgttacaggccaggctggaacaaattcggtatc |
| | aaattctgagccggtcaattgaattaaataaacttcctgaactgaaactt |
| | cctgatgagccttattttcagaatgtatctgaagaggaagtactgcgttt |
| | aacttctttgataaaagaacagttttccacatggcaaaatcagaagtatc |
| | aaaaagaactgaatctggataagaaaagagcagagcgattaacaatactt |
| | gcccgtataaaccgttatgaaaatgtatcgagggttgaaaaaagccgtct |
| | ggatgatttcaggagcctgttgcataaacaggcaattgcaaaacatgctg |
| | tacttgagcaggagaataaatatgttgaggcagcaaatgaattacgggtt |
| | tataaatcgcaactggagcaaattgagagtgagatattgtctgcaaaaga |
| | agaatatcagcttgtcacgcagctttttaaaaatgaaattttagacaagc |
| | taagacaaacaacagacagcattgagttattaactctggagttagagaaa |
| | aatgaagagcgtcaacaggcttcagtaatcagggcccctgtttcgggaaa |
| | agttcagcaactgaaggttcatactgaaggtggggttgttacaacagcgg |
| | aaacactgatggtcatcgttccggaagatgacacgctggaggttactgct |
| | ctggtacaaaataaagatattggttttattaacgtcgggcagaatgccat |
| | cattaaagtggaggcttttccttacacccgatatggttatctggtgggta |
| | aggtaaaaaatataaatttagatgcaatagaagaccagaaactgggactc |
| | gtttttaatgtcattgtttctgttgaagagaatgatttgtcaaccgggaa |
| | taagcacattccattaagctcgggtatggctgtcactgcagaaataaaga |
| | ctggaatgcgaagcgtaatcagttatcttcttagtcccctggaagaatct |
| | gtaacagaaagtttacatgagcgttaagtctcagagcagcggtatccggc |
| | tcatatcttctcctgtcagatccaagggcgaattccagcacactggcggc |
| | cgttactagtggatccaccggtctcgaggggcgcgccggtaccgaattct |
| | aattaatgggcatcatttttcagctatttcatttataaaataagttatga |
| | aaaaaatcatcatattactaacgacatttttcctgctttcgggatgcact |
| | attcccagggcggtatttaaatccagccttattaatcaggacgatcctcg |
| | ttataatctggtcgaagtcacgccgacattaaaactaagcgctcccgata |
| | ctgtgccgaaaactattgtcgatccggtttttgccgcaaataactggcac |
| | tggacatctttggctaaaggcgatgtgctgcatatcactattttatcctc |
| | gggcggggctggatatttatccaataacgcgagcggcgaccgtgcggatt |
| | ttgaaaatattcttgtgactgacagtaataccgttcaggtgccttatgcc |
| | gggacaatcccggtttctggattggatgtgacgcaactggctgatgagat |
| | caaaaagcgactttcgcgcgttgtcctgaatcctcaggtgattgtgacac |
| | ttaccgcccgcaccggagccatggtgacggtcgagggcagcgggaaaacg |
| | ggtcgataccctctcgaacagagtatgaatcgtctgagtcatcttttggc |
| | aacggcagtggcagtagaaaataccagcacagatatgatggaagttcacg |
| | tgacgcgccagcagcattatttcactgcgcgactgtcagatatttatcag |
| | tatcccggattggatattgccttacagccggatgaccgtatcactctgcg |
| | tcaagtgaccgagtatgttaatgtgctcggcgcagcaggtgttcagggga |
| | aacatgctctggttcagcgtcattccagcgtcgtggatgctttggcgctc |
| | gcgaaaggattaaatgacaatcttgccgatccgcaagcgatttttctgta |
| | caagcataacgaagcggaacaggcgaaacagcagatgcgtaagctgaata |
| | tctatcatgtcgatatgagccaacctaactcggtgtttttagcgcaggcc |
| | atacgagtggacaacggcgatgttatctatatctcgaacgcctctttgac |
| | tgatttcgctaaggtgaaagccgcattcgatagctttttgac |
| SEQ ID NO: 8 (Ty21a- | cccgttcctcattgatttgattgctaacgtcatgagcattactctatcat |
| eS-AR Construct) | tccagaatgcctcaatgaacaagttgtttgagaaagagtgtcgcaatgtt |
| | gcaaccagagcccttaaatatgtacgccagaagaaaactgaagggcgtct |
| | ggatgaagcattgtctgtattgattagcctgaaacgaattgagcctgatg |
| | tttctcgtctgatgcgtgaatataagcaaattatcagattatttaatgag |
| | tcacggaaggatggcggtagcactatcacgtcttatgaacatctagacta |
| | tgcgaaaaaattactcgtttttgatagcgaaaatgcctatgccttgaaat |
| | atgccgcattaaatgcaatgcatttacgcgactacacgcaggctttgcag |
| | tattggcagcgactggagaaagtgaatggaccaacggagccggtgacaag |
| | gcagatctcgacctgcataaccgcattacaaaaaaatacatcagggaagt |
| | cgtaacccggggtgtaggctggagctgcttcgaagttcctatactttcta |
| | gagaataggaacttcggaataggaacttcaagatcccctcacgctgccgc |
| | aagcactcagggcgcaagggctgctaaaggaagcggaacacgtagaaagc |
| | cagtccgcagaaacggtgctgaccccggatgaatgtcagctactgggcta |
| | tctggacaagggaaaacgcaagcgcaaagagaaagcaggtagcttgcagt |
| | gggcttacatggcgatagctagactgggcggttttatggacagcaagcga |
| | accggaattgccagctggggcgccctctggtaaggttgggaagccctgca |
| | aagtaaactggatggctttcttgccgccaaggatctgatggcgcagggga |
| | tcaagatctgatcaagagacaggatgaggatcgtttcgcatgattgaaca |
| | agatggattgcacgcaggttctccggccgcttgggtggagaggctattcg |
| | gctatgactgggcacaacagacaatcggctgctctgatgccgccgtgttc |
| | cggctgtcagcgcaggggcgcccggttctttttgtcaagaccgacctgtc |
| | cggtgccctgaatgaactgcaggacgaggcagcgcggctatcgtggctgg |
| | ccacgacgggcgttccttgcgcagctgtgctcgacgttgtcactgaagcg |
| | ggaagggactggctgctattgggcgaagtgccggggcaggatctcctgtc |
| | atctcaccttgctcctgccgagaaagtatccatcatggctgatgcaatgc |
| | ggcggctgcatacgcttgatccggctacctgcccattcgaccaccaagcg |
| | aaacatcgcatcgagcgagcacgtactcggatggaagccggtcttgtcga |
| | tcaggatgatctggacgaagagcatcaggggctcgcgccagccgaactgt |
| | tcgccaggctcaaggcgcgcatgcccgacggcgaggatctcgtcgtgacc |
| | catggcgatgcctgcttgccgaatatcatggtggaaaatggccgcttttc |
| | tggattcatcgactgtggccggctgggtgtggcggaccgctatcaggaca |
| | tagcgttggctacccgtgatattgctgaagagcttggcggcgaatgggct |
| | gaccgcttcctcgtgctttacggtatcgccgctcccgattcgcagcgcat |
| | cgccttctatcgccttcttgacgagttcttctgagcgggactctggggtt |
| | cgaaatgaccgaccaagcgacgcccaacctgccatcacgagatttcgatt |
| | ccaccgccgccttctatgaaaggttgggcttcggaatcgttttccgggac |
| | gccggctggatgatcctccagcgcggggatctcatgctggagttcttcgc |
| | ccaccccagcttcaaaagcgctctgaagttcctatactttctagagaata |
| | ggaacttcggaataggaactaaggaggatattcatatatgcattaatgtc |
| | taacaattcgttcaagccgacgccgcttcgcggcgcggcttaactcaagc |
| | gttagatgcactaagcacataattgctcacagccaaactatcaggtcaag |
| | tctgcttttattatttttaagcgtgcataataagccctacacaaattggg |
| | agatatatcatgaaaggctggctttttcttgttatcgcaatagttgCGAT |
| | CGTTATGACAACTTGACGGCTACATCATTCACTTTTTCTTCACAACCGGC |
| | ACGGAACTCGCTCGGGCTGGCCCCGGTGCATTTTTTAAATACCCGCGAGA |
| | AGTAGAGTTGATCGTCAAAACCAACATTGCGACCGACGGTGGCGATAGGC |
| | ATCCGGGTGGTGCTCAAAAGCAGCTTCGCCTGGCTGATACGTTGGTCCTC |
| | GCGCCAGCTTAAGACGCTAATCCCTAACTGCTGGCGGAAAAGATGTGACA |
| | GACGCGACGGCGACAAGCAAACATGCTGTGCGACGCTGGCGATATCAAAA |
| | TTGCTGTCTGCCAGGTGATCGCTGATGTACTGACAAGCCTCGCGTACCCG |
| | ATTATCCATCGGTGGATGGAGCGACTCGTTAATCGCTTCCATGCGCCGCA |
| | GTAACAATTGCTCAAGCAGATTTATCGCCAGCAGCTCCGAATAGCGCCCT |
| | TCCCCTTGCCCGGCGTTAATGATTTGCCCAAACAGGTCGCTGAAATGCGG |
| | CTGGTGCGCTTCATCCGGGCGAAAGAACCCCGTATTGGCAAATATTGACG |
| | GCCAGTTAAGCCATTCATGCCAGTAGGCGCGCGGACGAAAGTAAACCCAC |
| | TGGTGATACCATTCGCGAGCCTCCGGATGACGACCGTAGTGATGAATCTC |
| | TCCTGGCGGGAACAGCAAAATAACACCCGGTCGGCAAACAAATTCTCGTC |
| | CCTGATTTTTCACCACCCCCTGACCGCGAATGGTGAGATTGAGAATATAA |
| | CCTTTCATTCCCAGCGGTCGGTCGATAAAAAAATCGAGATAACCGTTGGC |
| | CTCAATCGGCGTTAAACCCGCCACCAGATGGGCATTAAACGAGTATCCCG |
| | GCAGCAGGGGATCATTTTGCGCTTCAGCCATACTTTTCATACTCCCGCCA |
| | TTCAGAGAAGAAACCAATTGTCCATATTGCATCAGACATTGCCGTCACTG |
| | CGTCTTTTACTGGCTCTTCTCGCTAACCAAACCGGTAACCCCGCTTATTA |
| | AAAGCATTCTGTAACAAAGCGGGACCAAAGCCATGACAAAAACGCGTAAC |
| | AAAAGTGTCTATAATCACGGCAGAAAAGTCCACATTGATTATTTGCACGG |
| | CGTCACACTTTGCTATGCCATAGCATTTTTATCCATAAGATTAGCGGATC |
| | CTACCTGACGCTTTTTATCGCAACTCTCTACTGTTTCTCCATACCCGTTT |
| | TTTTGGGAATTCGAGCTCGGAGTTAACAAAAGATGTTAGATGCAAACAAA |
| | TTACAGCAGGCAGTGGATCAGGCTTACACCCAATTTCACTCACTTAACGG |
| | CGGACAAAATGCCGATTACATTCCCTTTCTGGCGAATGTACCAGGTCAAC |
| | TGGCGGCAGTGGCTATCGTGACNTGCGATGGCAACGTCTATAGTGCGGGT |
| | GACAGTGATTACCGCTTTGCACTGGAATCCATCTCTAAAGTCTGTACGTT |
| | AGCCCTTGCGTTAGAAGATGTCGGCCCGCAGGCGGTACAGGACAAAATTG |
| | GCGCTGACCCGACCGGATTGCCCTTTAACTCAGTTATCGCCTTAGAGTTG |
| | CATGGCGGCAAACCGCTGTCGCCACTGGTAAATGCTGGCGCTATTGCCAC |
| | CACCAGCCTGATTAACGCTGAAAATGTTGAACAACGCTGGCAGCGAATTT |
| | TACATATCCAACAGCAACTGGCTGGTGAGCAGGTAGCGCTCTCTGACGAA |
| | GTCAACCAGTCGGAACAAACAACCAACTTCCATAACCGGGCCATTGCCTG |
| | GCTGCTGTACTCCGCCGGATATCTCTATTGTGATGCAATGGAAGCCTGTG |
| | ACGTGTACACCCGTCAGTGCTCTACGCTCATCAATACTGTTGAACTGGCA |
| | ACGCTTGGCGCGACGCTGGCGGCGGGTGGTGTGAATCCGTTGACGCATAA |
| | ACGCGTTCTTCAGGCCGACAACGTGCCGTACATTCTGGCCGAAATGATGA |
| | TGGAAGGGCTGTATGGTCGCTCCGGTGACTGGGCGTATCGCGTTGGTTTA |
| | CCGGGCAAAAGCGGTGTAGGTGGCGGTATTCTGGCGGTCGTCCCTGGCGT |
| | GATGGGAATTGCCGCGTTCTCACCACCGCTGGACGAAGAAGGCAACAGTG |
| | TTCGCGGTCAAAAAATGGTGGCATCGGTCGCTAAGCAACTCGGCTATAAC |
| | GTGTTTAAGGGCTGACCATGGAGTGTTTTAATGCGATCCAATCATTTTAA |
| | GGAGTTTAAAATGGATAAGAAGCAAGTAACGGATTTAAGGTCGGAACTAC |
| | TCGATTCACGTTTTGGTGCGAAGTCTATTTCCACTATCGCAGAATCAAAA |
| | CGTTTTCCGCTGCACGAAATGCGCGACGATGTCGCATTTCAGATTATCAA |
| | TGATGAATTATATCTTGATGGCAACGCTCGTCAGAACCTGGCCACTTTCT |
| | GCCAGACCTGGGACGACGAAAACGTCCACAAGTTGATGGATTTATCCATT |
| | AACAAAAACTGGATCGACAAAGAAGAATATCCGCAATCCGCAGCCATCGA |
| | CCTGCGTTGCGTAAACATGGTTGCCGATCTGTGGCATGCGCCTGCGCCGA |
| | AAAATGGTCAGGCCGTTGGCACCAACACCATTGGTTCTTCCGAGGCCTGT |
| | ATGCTCGGCGGGATGGCGATGAAATGGCGTTGGCGCAAGCGTATGGAAGC |
| | TGCAGGCAAACCAACGGATAAACCAAACCTGGTGTGCGGTCCGGTGCAAA |
| | TCTGCTGGCATAAATTCGCCCGCTACTGGGATGTGGAGCTGCGTGAGATC |
| | CCTATGCGCCCCGGTCAGTTGTTTATGGACCCGAAACGCATGATTGAAGC |
| | CTGCGACGAAAATACCATCGGCGTGGTGCCGACTTTCGGCGTGACCTACA |
| | CCGGTAACTATGAGTTCCCGCAACCGCTGCACGATGCACTGGATAAATTC |
| | CAGGCCGACACCGGTATCGACATCGACATGCACATCGACGCCGCCAGCGG |
| | TGGCTTTCTGGCACCGTTCGTCGCCCCGGATATCGTCTGGGACTTCCGCC |
| | TGCCGCGTGTGAAATCGATCAGTGCTTCAGGCCATAAATTCGGTCTGGCT |
| | CCGCTGGGCTGCGGCTGGGTTATCTGGCGTGACGAAGAAGCGCTGCCGCA |
| | GGAACTGGTGTTCAACGTTGACTACCTCGGTGGTCAGATTGGTACTTTTG |
| | CCATCAACTTCTCCCGCCCGGCGGGTCAGGTGATTGCACAGTACTATGAA |
| | TTCCTGCGCCTTGGTCGTGAAGGCTATACCAAAGTACAGAATGCTTCCTA |
| | CCAGGTCGCTGCCTATCTGGCGGATGAGATCGCCAAACTGGGGCCGTATG |
| | AGTTCATCTGTACCGGTCGCCCGGACGAAGGCATCCCGGCGGTTTGCTTC |
| | AAACTGAAAGATGGTGAAGATCCGGGATACACCCTGTACGACCTCTCTGA |
| | ACGTCTGCGTCTGCGCGGCTGGCAGGTTCCGGCCTTCACTCTCGGCGGTG |
| | AAGCCACCGACATCGTGGTGATGCGCATTATGTGTCGTCGCGGCTTCGAA |
| | ATGGACTTTGCTGAACTGTTGCTGGAAGACTACAAAGCCTCCCTGAAATA |
| | TCTCAGCGATCACCCGAAACTGCAGGGTATTGCCCAGCAGAACAGCTTTA |
| | AACATACCTGATAACCGTTTTGGAGTGATAATATGGCTACATCAGTACAG |
| | ACAGGTAAAGCTAAGCAGCTCACATTACTCGGATTCTTTGCCATAACGGC |
| | ATCGATGGTAATGGCTGTTTATGAATACCCTACCTTCGCAACATCGGGCT |
| | TTTCATTAGTCTTCTTCCTGCTATTAGGCGGGATTTTATGGTTTATTCCC |
| | GTGGGACTTTGTGCTGCGGAAATGGCCACCGTCGACGGCTGGGAAGAAGG |
| | TGGTGTCTTCGCCTGGGTATCAAATACTCTGGGTCCGAGATGGGGATTTG |
| | CAGCGATCTCATTTGGCTATCTGCAAATCGCCATTGGTTTTATTCCGATG |
| | CTCTATTTCGTGTTAGGGGCACTCTCCTACATCCTGAAATGGCCAGCGCT |
| | GAATGAAGACCCCATTACCAAAACTATTGCAGCACTCATCATTCTTTGGG |
| | CGCTGGCATTAACGCAGTTTGGTGGCACGAAATACACGGCGCGAATTGCT |
| | AAAGTTGGCTTCTTCGCCGGTATCCTGTTACCTGCATTTATTTTGATCGC |
| | ATTAGCGGCTATTTACCTGCACTCCGGTGCCCCCGTTGCTATCGAAATGG |
| | ATTCGAAGACCTTCTTCCCTGACTTCTCTAAAGTGGGCACACTGGTTGTA |
| | TTTGTTGCCTTCATTTTGAGTTATATGGGCGTAGAAGCTTCCGCAACTCA |
| | CGTCAATGAAATGAGCAACCCCGGGCGCGACTATCCACTGGCTATGTTAC |
| | TGCTGATGGTGGCGGCAATCTGCTTAAGCTCTGTTGGCGGTTTGTCTATT |
| | GCGATGGTCATTCCGGGTAATGAAATCAACCTCTCCGCAGGGGTAATGCA |
| | AACCTTTACCGTTCTGATGTCCCATGTGGCACCGGAAATTGAGTGGACGG |
| | TTCGCGTGATCTCCGCACTGCTGTTGCTGGGTGTTCTGGCGGAAATCGCC |
| | TCCTGGATTGTTGGTCCTTCTCGCGGGATGTATGTCACAGCGCAGAAAAA |
| | CCTGCTGCCAGCGGCATTCGCTAAAATGAACAAAAATGGCGTACCGGTAA |
| | CGCTGGTCATTTCGCAGCTGGTGATTACTTCTATCGCGTTGATCATCCTC |
| | ACCAATACCGGTGGCGGTAACAACATGTCCTTCCTGATCGCACTGGCGCT |
| | GACGGTGGTGATTTATCTGTGTGCTTATTTCATGCTGTTTATTGGCTACA |
| | TTGTGTTGGTTCTTAAACATCCTGACTTAAAACGCACATTTAATATCCCT |
| | GGTGGTAAAGGGGTGAAACTGGTCGTGGCAATTGTCGGTCTGCTGACTTC |
| | AATTATGGCGTTTATTGTTTCCTTCCTGCCGCCGGATAACATCCAGGGTG |
| | ATTCTACCGATATGTATGTTGAATTACTGGTTGTTAGTTTCCTGGTGGTA |
| | CTTGCCCTGCCCTTTATTCTCTATGCTGTTCATGATCGTAAAGGCAAAGC |
| | GAATACCGGCGTCACTCTGGAGCCAATCAACAGTCAGAACGCACCAAAAG |
| | GTCACTTCTTCCTGCACCCGCGTGCACGTTCACCACACTATATTGTGATG |
| | AATGACAAGAAACACTAACGATCGgcgaagtaatcgcaacatccgcatta |
| | aaatctagcgagggctttactgtcgaccaaaaaaatattgacaacataaa |
| | aaactttgtgttatacttgtaacgGactctagagggtattaataatgaaa |
| | ggagaggacatGAGCTCATGTTTGTGTTTCTGGTGCTGCTGCCGCTGGTG |
| | AGCAGCCAGTGCGTGAACCTGACCACCCGCACCCAGCTGCCGCCGGCGTA |
| | TACCAACAGCTTTACCCGCGGCGTGTATTATCCGGATAAAGTGTTTCGCA |
| | GCAGCGTGCTGCATAGCACCCAGGATCTGTTTCTGCCGTTTTTTAGCAAC |
| | GTGACCTGGTTTCATGCGATTCATGTGAGCGGCACCAACGGCACCAAACG |
| | CTTTGATAACCCGGTGCTGCCGTTTAACGATGGCGTGTATTTTGCGAGCA |
| | CCGAAAAAAGCAACATTATTCGCGGCTGGATTTTTGGCACCACCCTGGAT |
| | AGCAAAACCCAGAGCCTGCTGATTGTGAACAACGCGACCAACGTGGTGAT |
| | TAAAGTGTGCGAATTTCAGTTTTGCAACGATCCGTTTCTGGGCGTGTATT |
| | ATCATAAAAACAACAAAAGCTGGATGGAAAGCGAATTTCGCGTGTATAGC |
| | AGCGCGAACAACTGCACCTTTGAATATGTGAGCCAGCCGTTTCTGATGGA |
| | TCTGGAAGGCAAACAGGGCAACTTTAAAAACCTGCGCGAATTTGTGTTTA |
| | AAAACATTGATGGCTATTTTAAAATTTATAGCAAACATACCCCGATTAAC |
| | CTGGTGCGCGATCTGCCGCAGGGCTTTAGCGCGCTGGAACCGCTGGTGGA |
| | TCTGCCGATTGGCATTAACATTACCCGCTTTCAGACCCTGCTGGCGCTGC |
| | ATCGCAGCTATCTGACCCCGGGCGATAGCAGCAGCGGCTGGACCGCGGGC |
| | GCGGCGGCGTATTATGTGGGCTATCTGCAGCCGCGCACCTTTCTGCTGAA |
| | ATATAACGAAAACGGCACCATTACCGATGCGGTGGATTGCGCGCTGGATC |
| | CGCTGAGCGAAACCAAATGCACCCTGAAAAGCTTTACCGTGGAAAAAGGC |
| | ATTTATCAGACCAGCAACTTTCGCGTGCAGCCGACCGAAAGCATTGTGCG |
| | CTTTCCGAACATTACCAACCTGTGCCCGTTTGGCGAAGTGTTTAACGCGA |
| | CCCGCTTTGCGAGCGTGTATGCGTGGAACCGCAAACGCATTAGCAACTGC |
| | GTGGCGGATTATAGCGTGCTGTATAACAGCGCGAGCTTTAGCACCTTTAA |
| | ATGCTATGGCGTGAGCCCGACCAAACTGAACGATCTGTGCTTTACCAACG |
| | TGTATGCGGATAGCTTTGTGATTCGCGGCGATGAAGTGCGCCAGATTGCG |
| | CCGGGCCAGACCGGCAAAATTGCGGATTATAACTATAAACTGCCGGATGA |
| | TTTTACCGGCTGCGTGATTGCGTGGAACAGCAACAACCTGGATAGCAAAG |
| | TGGGCGGCAACTATAACTATCTGTATCGCCTGTTTCGCAAAAGCAACCTG |
| | AAACCGTTTGAACGCGATATTAGCACCGAAATTTATCAGGCGGGCAGCAC |
| | CCCGTGCAACGGCGTGGAAGGCTTTAACTGCTATTTTCCGCTGCAGAGCT |
| | ATGGCTTTCAGCCGACCAACGGCGTGGGCTATCAGCCGTATCGCGTGGTG |
| | GTGCTGAGCTTTGAACTGCTGCATGCGCCGGCGACCGTGTGCGGCCCGAA |
| | AAAAAGCACCAACCTGGTGAAAAACAAATGCGTGAACTTTAACTTTAACG |
| | GCCTGACCGGCACCGGCGTGCTGACCGAAAGCAACAAAAAATTTCTGCCG |
| | TTTCAGCAGTTTGGCCGCGATATTGCGGATACCACCGATGCGGTGCGCGA |
| | TCCGCAGACCCTGGAAATTCTGGATATTACCCCGTGCAGCTTTGGCGGCG |
| | TGAGCGTGATTACCCCGGGCACCAACACCAGCAACCAGGTGGCGGTGCTG |
| | TATCAGGGCGTGAACTGCACCGAAGTGCCGGTGGCGATTCATGCGGATCA |
| | GCTGACCCCGACCTGGCGCGTGTATAGCACCGGCAGCAACGTGTTTCAGA |
| | CCCGCGCGGGCTGCCTGATTGGCGCGGAACATGTGAACAACAGCTATGAA |
| | TGCGATATTCCGATTGGCGCGGGCATTTGCGCGAGCTATCAGACCCAGAC |
| | CAACAGCCCGGGCAGCGCGAGCAGCGTGGCGAGCCAGAGCATTATTGCGT |
| | ATACCATGAGCCTGGGCGCGGAAAACAGCGTGGCGTATAGCAACAACAGC |
| | ATTGCGATTCCGACCAACTTTACCATTAGCGTGACCACCGAAATTCTGCC |
| | GGTGAGCATGACCAAAACCAGCGTGGATTGCACCATGTATATTTGCGGCG |
| | ATAGCACCGAATGCAGCAACCTGCTGCTGCAGTATGGCAGCTTTTGCACC |
| | CAGCTGAACCGCGCGCTGACCGGCATTGCGGTGGAACAGGATAAAAACAC |
| | CCAGGAAGTGTTTGCGCAGGTGAAACAGATTTATAAAACCCCGCCGATTA |
| | AAGATTTTGGCGGCTTTAACTTTAGCCAGATTCTGCCGGATCCGAGCAAA |
| | CCGAGCAAACGCAGCTTTATTGAAGATCTGCTGTTTAACAAAGTGACCCT |
| | GGCGGATGCGGGCTTTATTAAACAGTATGGCGATTGCCTGGGCGATATTG |
| | CGGCGCGCGATCTGATTTGCGCGCAGAAATTTAACGGCCTGACCGTGCTG |
| | CCGCCGCTGCTGACCGATGAAATGATTGCGCAGTATACCAGCGCGCTGCT |
| | GGCGGGCACCATTACCAGCGGCTGGACCTTTGGCGCGGGCGCGGCGCTGC |
| | AGATTCCGTTTGCGATGCAGATGGCGTATCGCTTTAACGGCATTGGCGTG |
| | ACCCAGAACGTGCTGTATGAAAACCAGAAACTGATTGCGAACCAGTTTAA |
| | CAGCGCGATTGGCAAAATTCAGGATAGCCTGAGCAGCACCGCGAGCGCGC |
| | TGGGCAAACTGCAGGATGTGGTGAACCAGAACGCGCAGGCGCTGAACACC |
| | CTGGTGAAACAGCTGAGCAGCAACTTTGGCGCGATTAGCAGCGTGCTGAA |
| | CGATATTCTGAGCCGCCTGGATCCGCCGGAAGCGGAAGTGCAGATTGATC |
| | GCCTGATTACCGGCCGCCTGCAGAGCCTGCAGACCTATGTGACCCAGCAG |
| | CTGATTCGCGCGGCGGAAATTCGCGCGAGCGCGAACCTGGCGGCGACCAA |
| | AATGAGCGAATGCGTGCTGGGCCAGAGCAAACGCGTGGATTTTTGCGGCA |
| | AAGGCTATCATCTGATGAGCTTTCCGCAGAGCGCGCCGCATGGCGTGGTG |
| | TTTCTGCATGTGACCTATGTGCCGGCGCAGGAAAAAAACTTTACCACCGC |
| | GCCGGCGATTTGCCATGATGGCAAAGCGCATTTTCCGCGCGAAGGCGTGT |
| | TTGTGAGCAACGGCACCCATTGGTTTGTGACCCAGCGCAACTTTTATGAA |
| | CCGCAGATTATTACCACCGATAACACCTTTGTGAGCGGCAACTGCGATGT |
| | GGTGATTGGCATTGTGAACAACACCGTGTATGATCCGCTGCAGCCGGAAC |
| | TGGATAGCTTTAAAGAAGAACTGGATAAATATTTTAAAAACCATACCAGC |
| | CCGGATGTGGATCTGGGCGATATTAGCGGCATTAACGCGAGCGTGGTGAA |
| | CATTCAGAAAGAAATTGATCGCCTGAACGAAGTGGCGAAAAACCTGAACG |
| | AAAGCCTGATTGATCTGCAGGAACTGGGCAAATATGAACAGGGCTATATT |
| | CCGGAAGCGCCGCGCGATGGCCAGGCGTATGTGCGCAAAGATGGCGAATG |
| | GGTGCTGCTGAGCACCTTTCTGGCATTAGCCTATGGAAGTCAGGGTAATC |
| | TTAATCCATTAATTAATGAAATCAGCAAAATCATTTCAGCTGCAGGTAAT |
| | TTTGATGTTAAAGAGGAAAGAGCGGCCGCGTCTTTATTGCAGTTGTCCGG |
| | TAATGCCAGTGATTTTTCATATGGACGGAACTCAATAACTTTGACAGCAT |
| | CAGCATAAGAGCTCtttattaatttaaataatagcaatcttactgggctg |
| | tgccacataagattgctatttttttggagtcataatggattcttgtcata |
| | aaattgattatgggttatacgccctggagattttagcccaataccataac |
| | gtctctgttaacccggaagaaattaaacatagatttgacacagacgggac |
| | tggtctgggattaacgtcatggttgcttgctgcgaaatctttagaactaa |
| | aggtaaaacaggtaaaaaaaacaattgaccgattaaactttatttctctg |
| | cccgcattagtctggagagaggatggatgtcattttattctgactaaagt |
| | cagtaaagaagcaaacagatatcttatttttgatctggagcagcgaaatc |
| | cccgtgttctcgaacagtctgagtttgaggcgttatatcaggggcatatt |
| | attcttattgcttcccgttcttctgttaccgggaaactggcaaaatttga |
| | ctttacctggtttattcctgccattataaaatacaggaaaatatttattg |
| | aaacccttgttgtatctgtttttttacaattatttgcattaataaccccc |
| | cttttttttcaggtggttatggacaaagtattagtacacagggggttttc |
| | aacccttaatgttattactgtcgcattatctgttgtggtggtgtttgaga |
| | ttatactcagcggtttaagaacttacatttttgcacatagtacaagtcgg |
| | attgatgttgagttgggtgccaaactcttccggcatttactggcgctacc |
| | gatctcttattttgagagtcgtcgtgttggtgatactgttgccagggtaa |
| | gagaattagaccagatccgtaatttcctgacaggacaggcattaacatct |
| | gttctggacttattattttcattcatattttttgcggtaatgtggtatta |
| | cagcccaaagcttactctggtgatcttattttcgctgccctgttatgctg |
| | catggtctgtttttattagccccattttgcgacgtcgccttgatgataag |
| | ttttcacggaatgcggataatcaatctttcctggtggaatcagtcacggc |
| | gattaacactataaaagctatggcagtctcacctcagatgacgaacatat |
| | gggacaaacaattggcaggatatgttgctgcaggctttaaagtgacagta |
| | ttagccaccattggtcaacaaggaatacagttaatacaaaagactgttat |
| | gatcatcaacctgtggttgggtgcacacctggttatttccggggatttaa |
| | gtattggtcagttaattgcttttaatatgcttgcaggtcagattgttgca |
| | ccggttattcgccttgcacaaatctggcaggatttccagcaggttggtat |
| | atcagttacccgccttggtgatgtgcttaactctccaactgaaagttatc |
| | atgggaaactggcattaccggaaattaatggtgatatcacttttcgtaat |
| | atccggtttcgctataagcctgactctccggttattttagataatatcaa |
| | tctcagtattaagcagggggaggttattggtattgtcggacgttctggtt |
| | caggaaaaagcacattaactaaattaattcaacgtttttatattcctgaa |
| | aatggccaggtcttaattgatggacatgatcttgcgttggccgatcctaa |
| | ctggttacgtcgtcaggtgggggttgtgttgcaggacaatgtgctgctta |
| | atcgcagtattattgataatatttcactggctaatcctggcatgtccgtc |
| | gaaaaagttatttatgcagcgaaattagcaggtgctcatgattttatttc |
| | tgaattgcgtgaggggtataacaccattgtcggggaacagggggcaggat |
| | tatccggaggtcaacgtcaacgcatcgcaattgcaagggcgctggtgaac |
| | aaccctaaaatactcatctttgatgaagcaaccagtgctctggattatga |
| | gtcggagcatgtcatcatgcgcaatatgcacaaaatatgtaagggcagaa |
| | cggttataatcattgctcatcgtctgtctacagtaaaaaatgcagaccgc |
| | attattgtcatggaaaaagggaaaattgttgaacagggtaaacataagga |
| | gctgctttctgaaccggaaagtttatacagttacttatatcagttacagt |
| | cagactaacagaaagaacagaagaatatgaaaacatggttaatggggttc |
| | agcgagttcctgttgcgctataaacttgtctggagtgaaacatggaaaat |
| | ccggaagcagttagatactccggtacgtgaaaaggacgaaaatgaattct |
| | tacccgctcatctggaattaattgaaacgccggtatccagacggccgcgt |
| | ctggttgcttattttattatggggtttctggttattgctttcattttatc |
| | tgttttaggccaggtggaaattgttgccactgcaaatgggaaattaacac |
| | tcagtgggcgtagcaaagaaattaaacctattgaaaactcgatagttaaa |
| | gaaattatcgtaaaagaaggagagtcagtccggaaaggggatgtgttatt |
| | aaagcttacagcgctgggagctgaagctgatacgttaaaaacgcagtcat |
| | cactgttacaggccaggctggaacaaattcggtatcaaattctgagccgg |
| | tcaattgaattaaataaacttcctgaactgaaacttcctgatgagcctta |
| | ttttcagaatgtatctgaagaggaagtactgcgtttaacttctttgataa |
| | aagaacagttttccacatggcaaaatcagaagtatcaaaaagaactgaat |
| | ctggataagaaaagagcagagcgattaacaatacttgcccgtataaaccg |
| | ttatgaaaatgtatcgagggttgaaaaaagccgtctggatgatttcagga |
| | gcctgttgcataaacaggcaattgcaaaacatgctgtacttgagcaggag |
| | aataaatatgttgaggcagcaaatgaattacgggtttataaatcgcaact |
| | ggagcaaattgagagtgagatattgtctgcaaaagaagaatatcagcttg |
| | tcacgcagctttttaaaaatgaaattttagacaagctaagacaaacaaca |
| | gacagcattgagttattaactctggagttagagaaaaatgaagagcgtca |
| | acaggcttcagtaatcagggcccctgtttcgggaaaagttcagcaactga |
| | aggttcatactgaaggtggggttgttacaacagcggaaacactgatggtc |
| | atcgttccggaagatgacacgctggaggttactgctctggtacaaaataa |
| | agatattggttttattaacgtcgggcagaatgccatcattaaagtggagg |
| | cttttccttacacccgatatggttatctggtgggtaaggtaaaaaatata |
| | aatttagatgcaatagaagaccagaaactgggactcgtttttaatgtcat |
| | tgtttctgttgaagagaatgatttgtcaaccgggaataagcacattccat |
| | taagctcgggtatggctgtcactgcagaaataaagactggaatgcgaagc |
| | gtaatcagttatcttcttagtcccctggaagaatctgtaacagaaagttt |
| | acatgagcgttaagtctcagagcagcggtatccggctcatatcttctcct |
| | gtcagatccaagggcgaattccagcacactggcggccgttactagtggat |
| | ccaccggtctcgaggggcgcgccggtaccgaattctaattaatgggcatc |
| | atttttcagctatttcatttataaaataagttatgaaaaaaatcatcata |
| | ttactaacgacatttttcctgctttcgggatgcactattcccagggcggt |
| | atttaaatccagccttattaatcaggacgatcctcgttataatctggtcg |
| | aagtcacgccgacattaaaactaagcgctcccgatactgtgccgaaaact |
| | attgtcgatccggtttttgccgcaaataactggcactggacatctttggc |
| | taaaggcgatgtgctgcatatcactattttatcctcgggcggggctggat |
| | atttatccaataacgcgagcggcgaccgtgcggattttgaaaatattctt |
| | gtgactgacagtaataccgttcaggtgccttatgccgggacaatcccggt |
| | ttctggattggatgtgacgcaactggctgatgagatcaaaaagcgacttt |
| | cgcgcgttgtcctgaatcctcaggtgattgtgacacttaccgcccgcacc |
| | ggagccatggtgacggtcgagggcagcgggaaaacgggtcgataccctct |
| | cgaacagagtatgaatcgtctgagtcatcttttggcaacggcagtggcag |
| | tagaaaataccagcacagatatgatggaagttcacgtgacgcgccagcag |
| | cattatttcactgcgcgactgtcagatatttatcagtatcccggattgga |
| | tattgccttacagccggatgaccgtatcactctgcgtcaagtgaccgagt |
| | atgttaatgtgctcggcgcagcaggtgttcaggggaaacatgctctggtt |
| | cagcgtcattccagcgtcgtggatgctttggcgctcgcgaaaggattaaa |
| | tgacaatcttgccgatccgcaagcgatttttctgtacaagcataacgaag |
| | cggaacaggcgaaacagcagatgcgtaagctgaatatctatcatgtcgat |
| | atgagccaacctaactcggtgtttttagcgcaggccatacgagtggacaa |
| | cggcgatgttatctatatctcgaacgcctctttgactgatttcgctaagg |
| | tgaaagccgcattcgatagctttttgac |
| SEQ ID NO: 9 (Ty21a- | cccgttcctcattgatttgattgctaacgtcatgagcattactctatcat |
| M-N-AR Construct) | tccagaatgcctcaatgaacaagttgtttgagaaagagtgtcgcaatgtt |
| | gcaaccagagcccttaaatatgtacgccagaagaaaactgaagggcgtct |
| | ggatgaagcattgtctgtattgattagcctgaaacgaattgagcctgatg |
| | tttctcgtctgatgcgtgaatataagcaaattatcagattatttaatgag |
| | tcacggaaggatggcggtagcactatcacgtcttatgaacatctagacta |
| | tgcgaaaaaattactcgtttttgatagcgaaaatgcctatgccttgaaat |
| | atgccgcattaaatgcaatgcatttacgcgactacacgcaggctttgcag |
| | tattggcagcgactggagaaagtgaatggaccaacggagccggtgacaag |
| | gcagatctcgacctgcataaccgcattacaaaaaaatacatcagggaagt |
| | cgtaacccggggtgtaggctggagctgcttcgaagttcctatactttcta |
| | gagaataggaacttcggaataggaacttcaagatcccctcacgctgccgc |
| | aagcactcagggcgcaagggctgctaaaggaagcggaacacgtagaaagc |
| | cagtccgcagaaacggtgctgaccccggatgaatgtcagctactgggcta |
| | tctggacaagggaaaacgcaagcgcaaagagaaagcaggtagcttgcagt |
| | gggcttacatggcgatagctagactgggcggttttatggacagcaagcga |
| | accggaattgccagctggggcgccctctggtaaggttgggaagccctgca |
| | aagtaaactggatggctttcttgccgccaaggatctgatggcgcagggga |
| | tcaagatctgatcaagagacaggatgaggatcgtttcgcatgattgaaca |
| | agatggattgcacgcaggttctccggccgcttgggtggagaggctattcg |
| | gctatgactgggcacaacagacaatcggctgctctgatgccgccgtgttc |
| | cggctgtcagcgcaggggcgcccggttctttttgtcaagaccgacctgtc |
| | cggtgccctgaatgaactgcaggacgaggcagcgcggctatcgtggctgg |
| | ccacgacgggcgttccttgcgcagctgtgctcgacgttgtcactgaagcg |
| | ggaagggactggctgctattgggcgaagtgccggggcaggatctcctgtc |
| | atctcaccttgctcctgccgagaaagtatccatcatggctgatgcaatgc |
| | ggcggctgcatacgcttgatccggctacctgcccattcgaccaccaagcg |
| | aaacatcgcatcgagcgagcacgtactcggatggaagccggtcttgtcga |
| | tcaggatgatctggacgaagagcatcaggggctcgcgccagccgaactgt |
| | tcgccaggctcaaggcgcgcatgcccgacggcgaggatctcgtcgtgacc |
| | catggcgatgcctgcttgccgaatatcatggtggaaaatggccgcttttc |
| | tggattcatcgactgtggccggctgggtgtggcggaccgctatcaggaca |
| | tagcgttggctacccgtgatattgctgaagagcttggcggcgaatgggct |
| | gaccgcttcctcgtgctttacggtatcgccgctcccgattcgcagcgcat |
| | cgccttctatcgccttcttgacgagttcttctgagcgggactctggggtt |
| | cgaaatgaccgaccaagcgacgcccaacctgccatcacgagatttcgatt |
| | ccaccgccgccttctatgaaaggttgggcttcggaatcgttttccgggac |
| | gccggctggatgatcctccagcgcggggatctcatgctggagttcttcgc |
| | ccaccccagcttcaaaagcgctctgaagttcctatactttctagagaata |
| | ggaacttcggaataggaactaaggaggatattcatatatgcattaatgtc |
| | taacaattcgttcaagccgacgccgcttcgcggcgcggcttaactcaagc |
| | gttagatgcactaagcacataattgctcacagccaaactatcaggtcaag |
| | tctgcttttattatttttaagcgtgcataataagccctacacaaattggg |
| | agatatatcatgaaaggctggctttttcttgttatcgcaatagttgCGAT |
| | CGTTATGACAACTTGACGGCTACATCATTCACTTTTTCTTCACAACCGGC |
| | ACGGAACTCGCTCGGGCTGGCCCCGGTGCATTTTTTAAATACCCGCGAGA |
| | AGTAGAGTTGATCGTCAAAACCAACATTGCGACCGACGGTGGCGATAGGC |
| | ATCCGGGTGGTGCTCAAAAGCAGCTTCGCCTGGCTGATACGTTGGTCCTC |
| | GCGCCAGCTTAAGACGCTAATCCCTAACTGCTGGCGGAAAAGATGTGACA |
| | GACGCGACGGCGACAAGCAAACATGCTGTGCGACGCTGGCGATATCAAAA |
| | TTGCTGTCTGCCAGGTGATCGCTGATGTACTGACAAGCCTCGCGTACCCG |
| | ATTATCCATCGGTGGATGGAGCGACTCGTTAATCGCTTCCATGCGCCGCA |
| | GTAACAATTGCTCAAGCAGATTTATCGCCAGCAGCTCCGAATAGCGCCCT |
| | TCCCCTTGCCCGGCGTTAATGATTTGCCCAAACAGGTCGCTGAAATGCGG |
| | CTGGTGCGCTTCATCCGGGCGAAAGAACCCCGTATTGGCAAATATTGACG |
| | GCCAGTTAAGCCATTCATGCCAGTAGGCGCGCGGACGAAAGTAAACCCAC |
| | TGGTGATACCATTCGCGAGCCTCCGGATGACGACCGTAGTGATGAATCTC |
| | TCCTGGCGGGAACAGCAAAATAACACCCGGTCGGCAAACAAATTCTCGTC |
| | CCTGATTTTTCACCACCCCCTGACCGCGAATGGTGAGATTGAGAATATAA |
| | CCTTTCATTCCCAGCGGTCGGTCGATAAAAAAATCGAGATAACCGTTGGC |
| | CTCAATCGGCGTTAAACCCGCCACCAGATGGGCATTAAACGAGTATCCCG |
| | GCAGCAGGGGATCATTTTGCGCTTCAGCCATACTTTTCATACTCCCGCCA |
| | TTCAGAGAAGAAACCAATTGTCCATATTGCATCAGACATTGCCGTCACTG |
| | CGTCTTTTACTGGCTCTTCTCGCTAACCAAACCGGTAACCCCGCTTATTA |
| | AAAGCATTCTGTAACAAAGCGGGACCAAAGCCATGACAAAAACGCGTAAC |
| | AAAAGTGTCTATAATCACGGCAGAAAAGTCCACATTGATTATTTGCACGG |
| | CGTCACACTTTGCTATGCCATAGCATTTTTATCCATAAGATTAGCGGATC |
| | CTACCTGACGCTTTTTATCGCAACTCTCTACTGTTTCTCCATACCCGTTT |
| | TTTTGGGAATTCGAGCTCGGAGTTAACAAAAGATGTTAGATGCAAACAAA |
| | TTACAGCAGGCAGTGGATCAGGCTTACACCCAATTTCACTCACTTAACGG |
| | CGGACAAAATGCCGATTACATTCCCTTTCTGGCGAATGTACCAGGTCAAC |
| | TGGCGGCAGTGGCTATCGTGACNTGCGATGGCAACGTCTATAGTGCGGGT |
| | GACAGTGATTACCGCTTTGCACTGGAATCCATCTCTAAAGTCTGTACGTT |
| | AGCCCTTGCGTTAGAAGATGTCGGCCCGCAGGCGGTACAGGACAAAATTG |
| | GCGCTGACCCGACCGGATTGCCCTTTAACTCAGTTATCGCCTTAGAGTTG |
| | CATGGCGGCAAACCGCTGTCGCCACTGGTAAATGCTGGCGCTATTGCCAC |
| | CACCAGCCTGATTAACGCTGAAAATGTTGAACAACGCTGGCAGCGAATTT |
| | TACATATCCAACAGCAACTGGCTGGTGAGCAGGTAGCGCTCTCTGACGAA |
| | GTCAACCAGTCGGAACAAACAACCAACTTCCATAACCGGGCCATTGCCTG |
| | GCTGCTGTACTCCGCCGGATATCTCTATTGTGATGCAATGGAAGCCTGTG |
| | ACGTGTACACCCGTCAGTGCTCTACGCTCATCAATACTGTTGAACTGGCA |
| | ACGCTTGGCGCGACGCTGGCGGCGGGTGGTGTGAATCCGTTGACGCATAA |
| | ACGCGTTCTTCAGGCCGACAACGTGCCGTACATTCTGGCCGAAATGATGA |
| | TGGAAGGGCTGTATGGTCGCTCCGGTGACTGGGCGTATCGCGTTGGTTTA |
| | CCGGGCAAAAGCGGTGTAGGTGGCGGTATTCTGGCGGTCGTCCCTGGCGT |
| | GATGGGAATTGCCGCGTTCTCACCACCGCTGGACGAAGAAGGCAACAGTG |
| | TTCGCGGTCAAAAAATGGTGGCATCGGTCGCTAAGCAACTCGGCTATAAC |
| | GTGTTTAAGGGCTGACCATGGAGTGTTTTAATGCGATCCAATCATTTTAA |
| | GGAGTTTAAAATGGATAAGAAGCAAGTAACGGATTTAAGGTCGGAACTAC |
| | TCGATTCACGTTTTGGTGCGAAGTCTATTTCCACTATCGCAGAATCAAAA |
| | CGTTTTCCGCTGCACGAAATGCGCGACGATGTCGCATTTCAGATTATCAA |
| | TGATGAATTATATCTTGATGGCAACGCTCGTCAGAACCTGGCCACTTTCT |
| | GCCAGACCTGGGACGACGAAAACGTCCACAAGTTGATGGATTTATCCATT |
| | AACAAAAACTGGATCGACAAAGAAGAATATCCGCAATCCGCAGCCATCGA |
| | CCTGCGTTGCGTAAACATGGTTGCCGATCTGTGGCATGCGCCTGCGCCGA |
| | AAAATGGTCAGGCCGTTGGCACCAACACCATTGGTTCTTCCGAGGCCTGT |
| | ATGCTCGGCGGGATGGCGATGAAATGGCGTTGGCGCAAGCGTATGGAAGC |
| | TGCAGGCAAACCAACGGATAAACCAAACCTGGTGTGCGGTCCGGTGCAAA |
| | TCTGCTGGCATAAATTCGCCCGCTACTGGGATGTGGAGCTGCGTGAGATC |
| | CCTATGCGCCCCGGTCAGTTGTTTATGGACCCGAAACGCATGATTGAAGC |
| | CTGCGACGAAAATACCATCGGCGTGGTGCCGACTTTCGGCGTGACCTACA |
| | CCGGTAACTATGAGTTCCCGCAACCGCTGCACGATGCACTGGATAAATTC |
| | CAGGCCGACACCGGTATCGACATCGACATGCACATCGACGCCGCCAGCGG |
| | TGGCTTTCTGGCACCGTTCGTCGCCCCGGATATCGTCTGGGACTTCCGCC |
| | TGCCGCGTGTGAAATCGATCAGTGCTTCAGGCCATAAATTCGGTCTGGCT |
| | CCGCTGGGCTGCGGCTGGGTTATCTGGCGTGACGAAGAAGCGCTGCCGCA |
| | GGAACTGGTGTTCAACGTTGACTACCTCGGTGGTCAGATTGGTACTTTTG |
| | CCATCAACTTCTCCCGCCCGGCGGGTCAGGTGATTGCACAGTACTATGAA |
| | TTCCTGCGCCTTGGTCGTGAAGGCTATACCAAAGTACAGAATGCTTCCTA |
| | CCAGGTCGCTGCCTATCTGGCGGATGAGATCGCCAAACTGGGGCCGTATG |
| | AGTTCATCTGTACCGGTCGCCCGGACGAAGGCATCCCGGCGGTTTGCTTC |
| | AAACTGAAAGATGGTGAAGATCCGGGATACACCCTGTACGACCTCTCTGA |
| | ACGTCTGCGTCTGCGCGGCTGGCAGGTTCCGGCCTTCACTCTCGGCGGTG |
| | AAGCCACCGACATCGTGGTGATGCGCATTATGTGTCGTCGCGGCTTCGAA |
| | ATGGACTTTGCTGAACTGTTGCTGGAAGACTACAAAGCCTCCCTGAAATA |
| | TCTCAGCGATCACCCGAAACTGCAGGGTATTGCCCAGCAGAACAGCTTTA |
| | AACATACCTGATAACCGTTTTGGAGTGATAATATGGCTACATCAGTACAG |
| | ACAGGTAAAGCTAAGCAGCTCACATTACTCGGATTCTTTGCCATAACGGC |
| | ATCGATGGTAATGGCTGTTTATGAATACCCTACCTTCGCAACATCGGGCT |
| | TTTCATTAGTCTTCTTCCTGCTATTAGGCGGGATTTTATGGTTTATTCCC |
| | GTGGGACTTTGTGCTGCGGAAATGGCCACCGTCGACGGCTGGGAAGAAGG |
| | TGGTGTCTTCGCCTGGGTATCAAATACTCTGGGTCCGAGATGGGGATTTG |
| | CAGCGATCTCATTTGGCTATCTGCAAATCGCCATTGGTTTTATTCCGATG |
| | CTCTATTTCGTGTTAGGGGCACTCTCCTACATCCTGAAATGGCCAGCGCT |
| | GAATGAAGACCCCATTACCAAAACTATTGCAGCACTCATCATTCTTTGGG |
| | CGCTGGCATTAACGCAGTTTGGTGGCACGAAATACACGGCGCGAATTGCT |
| | AAAGTTGGCTTCTTCGCCGGTATCCTGTTACCTGCATTTATTTTGATCGC |
| | ATTAGCGGCTATTTACCTGCACTCCGGTGCCCCCGTTGCTATCGAAATGG |
| | ATTCGAAGACCTTCTTCCCTGACTTCTCTAAAGTGGGCACACTGGTTGTA |
| | TTTGTTGCCTTCATTTTGAGTTATATGGGCGTAGAAGCTTCCGCAACTCA |
| | CGTCAATGAAATGAGCAACCCCGGGCGCGACTATCCACTGGCTATGTTAC |
| | TGCTGATGGTGGCGGCAATCTGCTTAAGCTCTGTTGGCGGTTTGTCTATT |
| | GCGATGGTCATTCCGGGTAATGAAATCAACCTCTCCGCAGGGGTAATGCA |
| | AACCTTTACCGTTCTGATGTCCCATGTGGCACCGGAAATTGAGTGGACGG |
| | TTCGCGTGATCTCCGCACTGCTGTTGCTGGGTGTTCTGGCGGAAATCGCC |
| | TCCTGGATTGTTGGTCCTTCTCGCGGGATGTATGTCACAGCGCAGAAAAA |
| | CCTGCTGCCAGCGGCATTCGCTAAAATGAACAAAAATGGCGTACCGGTAA |
| | CGCTGGTCATTTCGCAGCTGGTGATTACTTCTATCGCGTTGATCATCCTC |
| | ACCAATACCGGTGGCGGTAACAACATGTCCTTCCTGATCGCACTGGCGCT |
| | GACGGTGGTGATTTATCTGTGTGCTTATTTCATGCTGTTTATTGGCTACA |
| | TTGTGTTGGTTCTTAAACATCCTGACTTAAAACGCACATTTAATATCCCT |
| | GGTGGTAAAGGGGTGAAACTGGTCGTGGCAATTGTCGGTCTGCTGACTTC |
| | AATTATGGCGTTTATTGTTTCCTTCCTGCCGCCGGATAACATCCAGGGTG |
| | ATTCTACCGATATGTATGTTGAATTACTGGTTGTTAGTTTCCTGGTGGTA |
| | CTTGCCCTGCCCTTTATTCTCTATGCTGTTCATGATCGTAAAGGCAAAGC |
| | GAATACCGGCGTCACTCTGGAGCCAATCAACAGTCAGAACGCACCAAAAG |
| | GTCACTTCTTCCTGCACCCGCGTGCACGTTCACCACACTATATTGTGATG |
| | AATGACAAGAAACACTAACGATCGgcgaagtaatcgcaacatccgcatta |
| | aaatctagcgagggctttactgtcgaccaaaaaaatattgacaacataaa |
| | aaactttgtgttatacttgtaacgGactctagagggtattaataatgaaa |
| | ggagaggacatGAGCTCCCTAGGATGAGCGATAACGGCCCGCAGAACCAG |
| | CGCAACGCGCCGCGCATTACCTTTGGCGGCCCGAGCGATAGCACCGGCAG |
| | CAACCAGAACGGCGAACGCAGCGGCGCGCGCAGCAAACAGCGCCGCCCGC |
| | AGGGCCTGCCGAACAACACCGCGAGCTGGTTTACCGCGCTGACCCAGCAT |
| | GGCAAAGAAGATCTGAAATTTCCGCGCGGCCAGGGCGTGCCGATTAACAC |
| | CAACAGCAGCCCGGATGATCAGATTGGCTATTATCGCCGCGCGACCCGCC |
| | GCATTCGCGGCGGCGATGGCAAAATGAAAGATCTGAGCCCGCGCTGGTAT |
| | TTTTATTATCTGGGCACCGGCCCGGAAGCGGGCCTGCCGTATGGCGCGAA |
| | CAAAGATGGCATTATTTGGGTGGCGACCGAAGGCGCGCTGAACACCCCGA |
| | AAGATCATATTGGCACCCGCAACCCGGCGAACAACGCGGCGATTGTGCTG |
| | CAGCTGCCGCAGGGCACCACCCTGCCGAAAGGCTTTTATGCGGAAGGCAG |
| | CCGCGGCGGCAGCCAGGCGAGCAGCCGCAGCAGCAGCCGCAGCCGCAACA |
| | GCAGCCGCAACAGCACCCCGGGCAGCAGCCGCGGCACCAGCCCGGCGCGC |
| | ATGGCGGGCAACGGCGGCGATGCGGCGCTGGCGCTGCTGCTGCTGGATCG |
| | CCTGAACCAGCTGGAAAGCAAAATGAGCGGCAAAGGCCAGCAGCAGCAGG |
| | GCCAGACCGTGACCAAAAAAAGCGCGGCGGAAGCGAGCAAAAAACCGCGC |
| | CAGAAACGCACCGCGACCAAAGCGTATAACGTGACCCAGGCGTTTGGCCG |
| | CCGCGGCCCGGAACAGACCCAGGGCAACTTTGGCGATCAGGAACTGATTC |
| | GCCAGGGCACCGATTATAAACATTGGCCGCAGATTGCGCAGTTTGCGCCG |
| | AGCGCGAGCGCGTTTTTTGGCATGAGCCGCATTGGCATGGAAGTGACCCC |
| | GAGCGGCACCTGGCTGACCTATACCGGCGCGATTAAACTGGATGATAAAG |
| | ATCCGAACTTTAAAGATCAGGTGATTCTGCTGAACAAACATATTGATGCG |
| | TATAAAACCTTTCCGCCGACCGAACCGAAAAAAGATAAAAAAAAAAAAGC |
| | GGATGAAACCCAGGCGCTGCCGCAGCGCCAGAAAAAACAGCAGACCGTGA |
| | CCCTGCTGCCGGCGGCGGATCTGGATGATTTTAGCAAACAGCTGCAGCAG |
| | AGCATGAGCAGCGCGGATAGCACCCAGGCGCCTAGGCTTAAGATGGCGGA |
| | TAGCAACGGCACCATTACCGTGGAAGAACTGAAAAAACTGCTGGAACAGT |
| | GGAACCTGGTGATTGGCTTTCTGTTTCTGACCTGGATTTGCCTGCTGCAG |
| | TTTGCGTATGCGAACCGCAACCGCTTTCTGTATATTATTAAACTGATTTT |
| | TCTGTGGCTGCTGTGGCCGGTGACCCTGGCGTGCTTTGTGCTGGCGGCGG |
| | TGTATCGCATTAACTGGATTACCGGCGGCATTGCGATTGCGATGGCGTGC |
| | CTGGTGGGCCTGATGTGGCTGAGCTATTTTATTGCGAGCTTTCGCCTGTT |
| | TGCGCGCACCCGCAGCATGTGGAGCTTTAACCCGGAAACCAACATTCTGC |
| | TGAACGTGCCGCTGCATGGCACCATTCTGACCCGCCCGCTGCTGGAAAGC |
| | GAACTGGTGATTGGCGCGGTGATTCTGCGCGGCCATCTGCGCATTGCGGG |
| | CCATCATCTGGGCCGCTGCGATATTAAAGATCTGCCGAAAGAAATTACCG |
| | TGGCGACCAGCCGCACCCTGAGCTATTATAAACTGGGCGCGAGCCAGCGC |
| | GTGGCGGGCGATAGCGGCTTTGCGGCGTATAGCCGCTATCGCATTGGCAA |
| | CTATAAACTGAACACCGATCATAGCAGCAGCAGCGATAACATTGCGCTGC |
| | TGGTGCAGCTTAAGGCATTAGCCTATGGAAGTCAGGGTAATCTTAATCCA |
| | TTAATTAATGAAATCAGCAAAATCATTTCAGCTGCAGGTAATTTTGATGT |
| | TAAAGAGGAAAGAGCGGCCGCGTCTTTATTGCAGTTGTCCGGTAATGCCA |
| | GTGATTTTTCATATGGACGGAACTCAATAACTTTGACAGCATCAGCATAA |
| | GAGCTCtttattaatttaaataatagcaatcttactgggctgtgccacat |
| | aagattgctatttttttggagtcataatggattcttgtcataaaattgat |
| | tatgggttatacgccctggagattttagcccaataccataacgtctctgt |
| | taacccggaagaaattaaacatagatttgacacagacgggactggtctgg |
| | gattaacgtcatggttgcttgctgcgaaatctttagaactaaaggtaaaa |
| | caggtaaaaaaaacaattgaccgattaaactttatttctctgcccgcatt |
| | agtctggagagaggatggatgtcattttattctgactaaagtcagtaaag |
| | aagcaaacagatatcttatttttgatctggagcagcgaaatccccgtgtt |
| | ctcgaacagtctgagtttgaggcgttatatcaggggcatattattcttat |
| | tgcttcccgttcttctgttaccgggaaactggcaaaatttgactttacct |
| | ggtttattcctgccattataaaatacaggaaaatatttattgaaaccctt |
| | gttgtatctgtttttttacaattatttgcattaataaccccccttttttt |
| | tcaggtggttatggacaaagtattagtacacagggggttttcaaccctta |
| | atgttattactgtcgcattatctgttgtggtggtgtttgagattatactc |
| | agcggtttaagaacttacatttttgcacatagtacaagtcggattgatgt |
| | tgagttgggtgccaaactcttccggcatttactggcgctaccgatctctt |
| | attttgagagtcgtcgtgttggtgatactgttgccagggtaagagaatta |
| | gaccagatccgtaatttcctgacaggacaggcattaacatctgttctgga |
| | cttattattttcattcatattttttgcggtaatgtggtattacagcccaa |
| | agcttactctggtgatcttattttcgctgccctgttatgctgcatggtct |
| | gtttttattagccccattttgcgacgtcgccttgatgataagttttcacg |
| | gaatgcggataatcaatctttcctggtggaatcagtcacggcgattaaca |
| | ctataaaagctatggcagtctcacctcagatgacgaacatatgggacaaa |
| | caattggcaggatatgttgctgcaggctttaaagtgacagtattagccac |
| | cattggtcaacaaggaatacagttaatacaaaagactgttatgatcatca |
| | acctgtggttgggtgcacacctggttatttccggggatttaagtattggt |
| | cagttaattgcttttaatatgcttgcaggtcagattgttgcaccggttat |
| | tcgccttgcacaaatctggcaggatttccagcaggttggtatatcagtta |
| | cccgccttggtgatgtgcttaactctccaactgaaagttatcatgggaaa |
| | ctggcattaccggaaattaatggtgatatcacttttcgtaatatccggtt |
| | tcgctataagcctgactctccggttattttagataatatcaatctcagta |
| | ttaagcagggggaggttattggtattgtcggacgttctggttcaggaaaa |
| | agcacattaactaaattaattcaacgtttttatattcctgaaaatggcca |
| | ggtcttaattgatggacatgatcttgcgttggccgatcctaactggttac |
| | gtcgtcaggtgggggttgtgttgcaggacaatgtgctgcttaatcgcagt |
| | attattgataatatttcactggctaatcctggcatgtccgtcgaaaaagt |
| | tatttatgcagcgaaattagcaggtgctcatgattttatttctgaattgc |
| | gtgaggggtataacaccattgtcggggaacagggggcaggattatccgga |
| | ggtcaacgtcaacgcatcgcaattgcaagggcgctggtgaacaaccctaa |
| | aatactcatctttgatgaagcaaccagtgctctggattatgagtcggagc |
| | atgtcatcatgcgcaatatgcacaaaatatgtaagggcagaacggttata |
| | atcattgctcatcgtctgtctacagtaaaaaatgcagaccgcattattgt |
| | catggaaaaagggaaaattgttgaacagggtaaacataaggagctgcttt |
| | ctgaaccggaaagtttatacagttacttatatcagttacagtcagactaa |
| | cagaaagaacagaagaatatgaaaacatggttaatggggttcagcgagtt |
| | cctgttgcgctataaacttgtctggagtgaaacatggaaaatccggaagc |
| | agttagatactccggtacgtgaaaaggacgaaaatgaattcttacccgct |
| | catctggaattaattgaaacgccggtatccagacggccgcgtctggttgc |
| | ttattttattatggggtttctggttattgctttcattttatctgttttag |
| | gccaggtggaaattgttgccactgcaaatgggaaattaacactcagtggg |
| | cgtagcaaagaaattaaacctattgaaaactcgatagttaaagaaattat |
| | cgtaaaagaaggagagtcagtccggaaaggggatgtgttattaaagctta |
| | cagcgctgggagctgaagctgatacgttaaaaacgcagtcatcactgtta |
| | caggccaggctggaacaaattcggtatcaaattctgagccggtcaattga |
| | attaaataaacttcctgaactgaaacttcctgatgagccttattttcaga |
| | atgtatctgaagaggaagtactgcgtttaacttctttgataaaagaacag |
| | ttttccacatggcaaaatcagaagtatcaaaaagaactgaatctggataa |
| | gaaaagagcagagcgattaacaatacttgcccgtataaaccgttatgaaa |
| | atgtatcgagggttgaaaaaagccgtctggatgatttcaggagcctgttg |
| | cataaacaggcaattgcaaaacatgctgtacttgagcaggagaataaata |
| | tgttgaggcagcaaatgaattacgggtttataaatcgcaactggagcaaa |
| | ttgagagtgagatattgtctgcaaaagaagaatatcagcttgtcacgcag |
| | ctttttaaaaatgaaattttagacaagctaagacaaacaacagacagcat |
| | tgagttattaactctggagttagagaaaaatgaagagcgtcaacaggctt |
| | cagtaatcagggcccctgtttcgggaaaagttcagcaactgaaggttcat |
| | actgaaggtggggttgttacaacagcggaaacactgatggtcatcgttcc |
| | ggaagatgacacgctggaggttactgctctggtacaaaataaagatattg |
| | gttttattaacgtcgggcagaatgccatcattaaagtggaggcttttcct |
| | tacacccgatatggttatctggtgggtaaggtaaaaaatataaatttaga |
| | tgcaatagaagaccagaaactgggactcgtttttaatgtcattgtttctg |
| | ttgaagagaatgatttgtcaaccgggaataagcacattccattaagctcg |
| | ggtatggctgtcactgcagaaataaagactggaatgcgaagcgtaatcag |
| | ttatcttcttagtcccctggaagaatctgtaacagaaagtttacatgagc |
| | gttaagtctcagagcagcggtatccggctcatatcttctcctgtcagatc |
| | caagggcgaattccagcacactggcggccgttactagtggatccaccggt |
| | ctcgaggggcgcgccggtaccgaattctaattaatgggcatcatttttca |
| | gctatttcatttataaaataagttatgaaaaaaatcatcatattactaac |
| | gacatttttcctgctttcgggatgcactattcccagggcggtatttaaat |
| | ccagccttattaatcaggacgatcctcgttataatctggtcgaagtcacg |
| | ccgacattaaaactaagcgctcccgatactgtgccgaaaactattgtcga |
| | tccggtttttgccgcaaataactggcactggacatctttggctaaaggcg |
| | atgtgctgcatatcactattttatcctcgggcggggctggatatttatcc |
| | aataacgcgagcggcgaccgtgcggattttgaaaatattcttgtgactga |
| | cagtaataccgttcaggtgccttatgccgggacaatcccggtttctggat |
| | tggatgtgacgcaactggctgatgagatcaaaaagcgactttcgcgcgtt |
| | gtcctgaatcctcaggtgattgtgacacttaccgcccgcaccggagccat |
| | ggtgacggtcgagggcagcgggaaaacgggtcgataccctctcgaacaga |
| | gtatgaatcgtctgagtcatcttttggcaacggcagtggcagtagaaaat |
| | accagcacagatatgatggaagttcacgtgacgcgccagcagcattattt |
| | cactgcgcgactgtcagatatttatcagtatcccggattggatattgcct |
| | tacagccggatgaccgtatcactctgcgtcaagtgaccgagtatgttaat |
| | gtgctcggcgcagcaggtgttcaggggaaacatgctctggttcagcgtca |
| | ttccagcgtcgtggatgctttggcgctcgcgaaaggattaaatgacaatc |
| | ttgccgatccgcaagcgatttttctgtacaagcataacgaagcggaacag |
| | gcgaaacagcagatgcgtaagctgaatatctatcatgtcgatatgagcca |
| | acctaactcggtgtttttagcgcaggccatacgagtggacaacggcgatg |
| | ttatctatatctcgaacgcctctttgactgatttcgctaaggtgaaagcc |
| | gcattcgatagctttttgac |
| SEQ ID NO: 10 (Ty21a- | cccgttcctcattgatttgattgctaacgtcatgagcattactctatcat |
| eS-M-N-AR Construct) | tccagaatgcctcaatgaacaagttgtttgagaaagagtgtcgcaatgtt |
| | gcaaccagagcccttaaatatgtacgccagaagaaaactgaagggcgtct |
| | ggatgaagcattgtctgtattgattagcctgaaacgaattgagcctgatg |
| | tttctcgtctgatgcgtgaatataagcaaattatcagattatttaatgag |
| | tcacggaaggatggcggtagcactatcacgtcttatgaacatctagacta |
| | tgcgaaaaaattactcgtttttgatagcgaaaatgcctatgccttgaaat |
| | atgccgcattaaatgcaatgcatttacgcgactacacgcaggctttgcag |
| | tattggcagcgactggagaaagtgaatggaccaacggagccggtgacaag |
| | gcagatctcgacctgcataaccgcattacaaaaaaatacatcagggaagt |
| | cgtaacccggggtgtaggctggagctgcttcgaagttcctatactttcta |
| | gagaataggaacttcggaataggaacttcaagatcccctcacgctgccgc |
| | aagcactcagggcgcaagggctgctaaaggaagcggaacacgtagaaagc |
| | cagtccgcagaaacggtgctgaccccggatgaatgtcagctactgggcta |
| | tctggacaagggaaaacgcaagcgcaaagagaaagcaggtagcttgcagt |
| | gggcttacatggcgatagctagactgggcggttttatggacagcaagcga |
| | accggaattgccagctggggcgccctctggtaaggttgggaagccctgca |
| | aagtaaactggatggctttcttgccgccaaggatctgatggcgcagggga |
| | tcaagatctgatcaagagacaggatgaggatcgtttcgcatgattgaaca |
| | agatggattgcacgcaggttctccggccgcttgggtggagaggctattcg |
| | gctatgactgggcacaacagacaatcggctgctctgatgccgccgtgttc |
| | cggctgtcagcgcaggggcgcccggttctttttgtcaagaccgacctgtc |
| | cggtgccctgaatgaactgcaggacgaggcagcgcggctatcgtggctgg |
| | ccacgacgggcgttccttgcgcagctgtgctcgacgttgtcactgaagcg |
| | ggaagggactggctgctattgggcgaagtgccggggcaggatctcctgtc |
| | atctcaccttgctcctgccgagaaagtatccatcatggctgatgcaatgc |
| | ggcggctgcatacgcttgatccggctacctgcccattcgaccaccaagcg |
| | aaacatcgcatcgagcgagcacgtactcggatggaagccggtcttgtcga |
| | tcaggatgatctggacgaagagcatcaggggctcgcgccagccgaactgt |
| | tcgccaggctcaaggcgcgcatgcccgacggcgaggatctcgtcgtgacc |
| | catggcgatgcctgcttgccgaatatcatggtggaaaatggccgcttttc |
| | tggattcatcgactgtggccggctgggtgtggcggaccgctatcaggaca |
| | tagcgttggctacccgtgatattgctgaagagcttggcggcgaatgggct |
| | gaccgcttcctcgtgctttacggtatcgccgctcccgattcgcagcgcat |
| | cgccttctatcgccttcttgacgagttcttctgagcgggactctggggtt |
| | cgaaatgaccgaccaagcgacgcccaacctgccatcacgagatttcgatt |
| | ccaccgccgccttctatgaaaggttgggcttcggaatcgttttccgggac |
| | gccggctggatgatcctccagcgcggggatctcatgctggagttcttcgc |
| | ccaccccagcttcaaaagcgctctgaagttcctatactttctagagaata |
| | ggaacttcggaataggaactaaggaggatattcatatatgcattaatgtc |
| | taacaattcgttcaagccgacgccgcttcgcggcgcggcttaactcaagc |
| | gttagatgcactaagcacataattgctcacagccaaactatcaggtcaag |
| | tctgcttttattatttttaagcgtgcataataagccctacacaaattggg |
| | agatatatcatgaaaggctggctttttcttgttatcgcaatagttgCGAT |
| | CGTTATGACAACTTGACGGCTACATCATTCACTTTTTCTTCACAACCGGC |
| | ACGGAACTCGCTCGGGCTGGCCCCGGTGCATTTTTTAAATACCCGCGAGA |
| | AGTAGAGTTGATCGTCAAAACCAACATTGCGACCGACGGTGGCGATAGGC |
| | ATCCGGGTGGTGCTCAAAAGCAGCTTCGCCTGGCTGATACGTTGGTCCTC |
| | GCGCCAGCTTAAGACGCTAATCCCTAACTGCTGGCGGAAAAGATGTGACA |
| | GACGCGACGGCGACAAGCAAACATGCTGTGCGACGCTGGCGATATCAAAA |
| | TTGCTGTCTGCCAGGTGATCGCTGATGTACTGACAAGCCTCGCGTACCCG |
| | ATTATCCATCGGTGGATGGAGCGACTCGTTAATCGCTTCCATGCGCCGCA |
| | GTAACAATTGCTCAAGCAGATTTATCGCCAGCAGCTCCGAATAGCGCCCT |
| | TCCCCTTGCCCGGCGTTAATGATTTGCCCAAACAGGTCGCTGAAATGCGG |
| | CTGGTGCGCTTCATCCGGGCGAAAGAACCCCGTATTGGCAAATATTGACG |
| | GCCAGTTAAGCCATTCATGCCAGTAGGCGCGCGGACGAAAGTAAACCCAC |
| | TGGTGATACCATTCGCGAGCCTCCGGATGACGACCGTAGTGATGAATCTC |
| | TCCTGGCGGGAACAGCAAAATAACACCCGGTCGGCAAACAAATTCTCGTC |
| | CCTGATTTTTCACCACCCCCTGACCGCGAATGGTGAGATTGAGAATATAA |
| | CCTTTCATTCCCAGCGGTCGGTCGATAAAAAAATCGAGATAACCGTTGGC |
| | CTCAATCGGCGTTAAACCCGCCACCAGATGGGCATTAAACGAGTATCCCG |
| | GCAGCAGGGGATCATTTTGCGCTTCAGCCATACTTTTCATACTCCCGCCA |
| | TTCAGAGAAGAAACCAATTGTCCATATTGCATCAGACATTGCCGTCACTG |
| | CGTCTTTTACTGGCTCTTCTCGCTAACCAAACCGGTAACCCCGCTTATTA |
| | AAAGCATTCTGTAACAAAGCGGGACCAAAGCCATGACAAAAACGCGTAAC |
| | AAAAGTGTCTATAATCACGGCAGAAAAGTCCACATTGATTATTTGCACGG |
| | CGTCACACTTTGCTATGCCATAGCATTTTTATCCATAAGATTAGCGGATC |
| | CTACCTGACGCTTTTTATCGCAACTCTCTACTGTTTCTCCATACCCGTTT |
| | TTTTGGGAATTCGAGCTCGGAGTTAACAAAAGATGTTAGATGCAAACAAA |
| | TTACAGCAGGCAGTGGATCAGGCTTACACCCAATTTCACTCACTTAACGG |
| | CGGACAAAATGCCGATTACATTCCCTTTCTGGCGAATGTACCAGGTCAAC |
| | TGGCGGCAGTGGCTATCGTGACNTGCGATGGCAACGTCTATAGTGCGGGT |
| | GACAGTGATTACCGCTTTGCACTGGAATCCATCTCTAAAGTCTGTACGTT |
| | AGCCCTTGCGTTAGAAGATGTCGGCCCGCAGGCGGTACAGGACAAAATTG |
| | GCGCTGACCCGACCGGATTGCCCTTTAACTCAGTTATCGCCTTAGAGTTG |
| | CATGGCGGCAAACCGCTGTCGCCACTGGTAAATGCTGGCGCTATTGCCAC |
| | CACCAGCCTGATTAACGCTGAAAATGTTGAACAACGCTGGCAGCGAATTT |
| | TACATATCCAACAGCAACTGGCTGGTGAGCAGGTAGCGCTCTCTGACGAA |
| | GTCAACCAGTCGGAACAAACAACCAACTTCCATAACCGGGCCATTGCCTG |
| | GCTGCTGTACTCCGCCGGATATCTCTATTGTGATGCAATGGAAGCCTGTG |
| | ACGTGTACACCCGTCAGTGCTCTACGCTCATCAATACTGTTGAACTGGCA |
| | ACGCTTGGCGCGACGCTGGCGGCGGGTGGTGTGAATCCGTTGACGCATAA |
| | ACGCGTTCTTCAGGCCGACAACGTGCCGTACATTCTGGCCGAAATGATGA |
| | TGGAAGGGCTGTATGGTCGCTCCGGTGACTGGGCGTATCGCGTTGGTTTA |
| | CCGGGCAAAAGCGGTGTAGGTGGCGGTATTCTGGCGGTCGTCCCTGGCGT |
| | GATGGGAATTGCCGCGTTCTCACCACCGCTGGACGAAGAAGGCAACAGTG |
| | TTCGCGGTCAAAAAATGGTGGCATCGGTCGCTAAGCAACTCGGCTATAAC |
| | GTGTTTAAGGGCTGACCATGGAGTGTTTTAATGCGATCCAATCATTTTAA |
| | GGAGTTTAAAATGGATAAGAAGCAAGTAACGGATTTAAGGTCGGAACTAC |
| | TCGATTCACGTTTTGGTGCGAAGTCTATTTCCACTATCGCAGAATCAAAA |
| | CGTTTTCCGCTGCACGAAATGCGCGACGATGTCGCATTTCAGATTATCAA |
| | TGATGAATTATATCTTGATGGCAACGCTCGTCAGAACCTGGCCACTTTCT |
| | GCCAGACCTGGGACGACGAAAACGTCCACAAGTTGATGGATTTATCCATT |
| | AACAAAAACTGGATCGACAAAGAAGAATATCCGCAATCCGCAGCCATCGA |
| | CCTGCGTTGCGTAAACATGGTTGCCGATCTGTGGCATGCGCCTGCGCCGA |
| | AAAATGGTCAGGCCGTTGGCACCAACACCATTGGTTCTTCCGAGGCCTGT |
| | ATGCTCGGCGGGATGGCGATGAAATGGCGTTGGCGCAAGCGTATGGAAGC |
| | TGCAGGCAAACCAACGGATAAACCAAACCTGGTGTGCGGTCCGGTGCAAA |
| | TCTGCTGGCATAAATTCGCCCGCTACTGGGATGTGGAGCTGCGTGAGATC |
| | CCTATGCGCCCCGGTCAGTTGTTTATGGACCCGAAACGCATGATTGAAGC |
| | CTGCGACGAAAATACCATCGGCGTGGTGCCGACTTTCGGCGTGACCTACA |
| | CCGGTAACTATGAGTTCCCGCAACCGCTGCACGATGCACTGGATAAATTC |
| | CAGGCCGACACCGGTATCGACATCGACATGCACATCGACGCCGCCAGCGG |
| | TGGCTTTCTGGCACCGTTCGTCGCCCCGGATATCGTCTGGGACTTCCGCC |
| | TGCCGCGTGTGAAATCGATCAGTGCTTCAGGCCATAAATTCGGTCTGGCT |
| | CCGCTGGGCTGCGGCTGGGTTATCTGGCGTGACGAAGAAGCGCTGCCGCA |
| | GGAACTGGTGTTCAACGTTGACTACCTCGGTGGTCAGATTGGTACTTTTG |
| | CCATCAACTTCTCCCGCCCGGCGGGTCAGGTGATTGCACAGTACTATGAA |
| | TTCCTGCGCCTTGGTCGTGAAGGCTATACCAAAGTACAGAATGCTTCCTA |
| | CCAGGTCGCTGCCTATCTGGCGGATGAGATCGCCAAACTGGGGCCGTATG |
| | AGTTCATCTGTACCGGTCGCCCGGACGAAGGCATCCCGGCGGTTTGCTTC |
| | AAACTGAAAGATGGTGAAGATCCGGGATACACCCTGTACGACCTCTCTGA |
| | ACGTCTGCGTCTGCGCGGCTGGCAGGTTCCGGCCTTCACTCTCGGCGGTG |
| | AAGCCACCGACATCGTGGTGATGCGCATTATGTGTCGTCGCGGCTTCGAA |
| | ATGGACTTTGCTGAACTGTTGCTGGAAGACTACAAAGCCTCCCTGAAATA |
| | TCTCAGCGATCACCCGAAACTGCAGGGTATTGCCCAGCAGAACAGCTTTA |
| | AACATACCTGATAACCGTTTTGGAGTGATAATATGGCTACATCAGTACAG |
| | ACAGGTAAAGCTAAGCAGCTCACATTACTCGGATTCTTTGCCATAACGGC |
| | ATCGATGGTAATGGCTGTTTATGAATACCCTACCTTCGCAACATCGGGCT |
| | TTTCATTAGTCTTCTTCCTGCTATTAGGCGGGATTTTATGGTTTATTCCC |
| | GTGGGACTTTGTGCTGCGGAAATGGCCACCGTCGACGGCTGGGAAGAAGG |
| | TGGTGTCTTCGCCTGGGTATCAAATACTCTGGGTCCGAGATGGGGATTTG |
| | CAGCGATCTCATTTGGCTATCTGCAAATCGCCATTGGTTTTATTCCGATG |
| | CTCTATTTCGTGTTAGGGGCACTCTCCTACATCCTGAAATGGCCAGCGCT |
| | GAATGAAGACCCCATTACCAAAACTATTGCAGCACTCATCATTCTTTGGG |
| | CGCTGGCATTAACGCAGTTTGGTGGCACGAAATACACGGCGCGAATTGCT |
| | AAAGTTGGCTTCTTCGCCGGTATCCTGTTACCTGCATTTATTTTGATCGC |
| | ATTAGCGGCTATTTACCTGCACTCCGGTGCCCCCGTTGCTATCGAAATGG |
| | ATTCGAAGACCTTCTTCCCTGACTTCTCTAAAGTGGGCACACTGGTTGTA |
| | TTTGTTGCCTTCATTTTGAGTTATATGGGCGTAGAAGCTTCCGCAACTCA |
| | CGTCAATGAAATGAGCAACCCCGGGCGCGACTATCCACTGGCTATGTTAC |
| | TGCTGATGGTGGCGGCAATCTGCTTAAGCTCTGTTGGCGGTTTGTCTATT |
| | GCGATGGTCATTCCGGGTAATGAAATCAACCTCTCCGCAGGGGTAATGCA |
| | AACCTTTACCGTTCTGATGTCCCATGTGGCACCGGAAATTGAGTGGACGG |
| | TTCGCGTGATCTCCGCACTGCTGTTGCTGGGTGTTCTGGCGGAAATCGCC |
| | TCCTGGATTGTTGGTCCTTCTCGCGGGATGTATGTCACAGCGCAGAAAAA |
| | CCTGCTGCCAGCGGCATTCGCTAAAATGAACAAAAATGGCGTACCGGTAA |
| | CGCTGGTCATTTCGCAGCTGGTGATTACTTCTATCGCGTTGATCATCCTC |
| | ACCAATACCGGTGGCGGTAACAACATGTCCTTCCTGATCGCACTGGCGCT |
| | GACGGTGGTGATTTATCTGTGTGCTTATTTCATGCTGTTTATTGGCTACA |
| | TTGTGTTGGTTCTTAAACATCCTGACTTAAAACGCACATTTAATATCCCT |
| | GGTGGTAAAGGGGTGAAACTGGTCGTGGCAATTGTCGGTCTGCTGACTTC |
| | AATTATGGCGTTTATTGTTTCCTTCCTGCCGCCGGATAACATCCAGGGTG |
| | ATTCTACCGATATGTATGTTGAATTACTGGTTGTTAGTTTCCTGGTGGTA |
| | CTTGCCCTGCCCTTTATTCTCTATGCTGTTCATGATCGTAAAGGCAAAGC |
| | GAATACCGGCGTCACTCTGGAGCCAATCAACAGTCAGAACGCACCAAAAG |
| | GTCACTTCTTCCTGCACCCGCGTGCACGTTCACCACACTATATTGTGATG |
| | AATGACAAGAAACACTAACGATCGgcgaagtaatcgcaacatccgcatta |
| | aaatctagcgagggctttactgtcgaccaaaaaaatattgacaacataaa |
| | aaactttgtgttatacttgtaacgGactctagagggtattaataatgaaa |
| | ggagaggacatATGAGCGATAACGGCCCGCAGAACCAGCGCAACGCGCCG |
| | CGCATTACCTTTGGCGGCCCGAGCGATAGCACCGGCAGCAACCAGAACGG |
| | CGAACGCAGCGGCGCGCGCAGCAAACAGCGCCGCCCGCAGGGCCTGCCGA |
| | ACAACACCGCGAGCTGGTTTACCGCGCTGACCCAGCATGGCAAAGAAGAT |
| | CTGAAATTTCCGCGCGGCCAGGGCGTGCCGATTAACACCAACAGCAGCCC |
| | GGATGATCAGATTGGCTATTATCGCCGCGCGACCCGCCGCATTCGCGGCG |
| | GCGATGGCAAAATGAAAGATCTGAGCCCGCGCTGGTATTTTTATTATCTG |
| | GGCACCGGCCCGGAAGCGGGCCTGCCGTATGGCGCGAACAAAGATGGCAT |
| | TATTTGGGTGGCGACCGAAGGCGCGCTGAACACCCCGAAAGATCATATTG |
| | GCACCCGCAACCCGGCGAACAACGCGGCGATTGTGCTGCAGCTGCCGCAG |
| | GGCACCACCCTGCCGAAAGGCTTTTATGCGGAAGGCAGCCGCGGCGGCAG |
| | CCAGGCGAGCAGCCGCAGCAGCAGCCGCAGCCGCAACAGCAGCCGCAACA |
| | GCACCCCGGGCAGCAGCCGCGGCACCAGCCCGGCGCGCATGGCGGGCAAC |
| | GGCGGCGATGCGGCGCTGGCGCTGCTGCTGCTGGATCGCCTGAACCAGCT |
| | GGAAAGCAAAATGAGCGGCAAAGGCCAGCAGCAGCAGGGCCAGACCGTGA |
| | CCAAAAAAAGCGCGGCGGAAGCGAGCAAAAAACCGCGCCAGAAACGCACC |
| | GCGACCAAAGCGTATAACGTGACCCAGGCGTTTGGCCGCCGCGGCCCGGA |
| | ACAGACCCAGGGCAACTTTGGCGATCAGGAACTGATTCGCCAGGGCACCG |
| | ATTATAAACATTGGCCGCAGATTGCGCAGTTTGCGCCGAGCGCGAGCGCG |
| | TTTTTTGGCATGAGCCGCATTGGCATGGAAGTGACCCCGAGCGGCACCTG |
| | GCTGACCTATACCGGCGCGATTAAACTGGATGATAAAGATCCGAACTTTA |
| | AAGATCAGGTGATTCTGCTGAACAAACATATTGATGCGTATAAAACCTTT |
| | CCGCCGACCGAACCGAAAAAAGATAAAAAAAAAAAAGCGGATGAAACCCA |
| | GGCGCTGCCGCAGCGCCAGAAAAAACAGCAGACCGTGACCCTGCTGCCGG |
| | CGGCGGATCTGGATGATTTTAGCAAACAGCTGCAGCAGAGCATGAGCAGC |
| | GCGGATAGCACCCAGGCGCCTAGGCTTAAGATGGCGGATAGCAACGGCAC |
| | CATTACCGTGGAAGAACTGAAAAAACTGCTGGAACAGTGGAACCTGGTGA |
| | TTGGCTTTCTGTTTCTGACCTGGATTTGCCTGCTGCAGTTTGCGTATGCG |
| | AACCGCAACCGCTTTCTGTATATTATTAAACTGATTTTTCTGTGGCTGCT |
| | GTGGCCGGTGACCCTGGCGTGCTTTGTGCTGGCGGCGGTGTATCGCATTA |
| | ACTGGATTACCGGCGGCATTGCGATTGCGATGGCGTGCCTGGTGGGCCTG |
| | ATGTGGCTGAGCTATTTTATTGCGAGCTTTCGCCTGTTTGCGCGCACCCG |
| | CAGCATGTGGAGCTTTAACCCGGAAACCAACATTCTGCTGAACGTGCCGC |
| | TGCATGGCACCATTCTGACCCGCCCGCTGCTGGAAAGCGAACTGGTGATT |
| | GGCGCGGTGATTCTGCGCGGCCATCTGCGCATTGCGGGCCATCATCTGGG |
| | CCGCTGCGATATTAAAGATCTGCCGAAAGAAATTACCGTGGCGACCAGCC |
| | GCACCCTGAGCTATTATAAACTGGGCGCGAGCCAGCGCGTGGCGGGCGAT |
| | AGCGGCTTTGCGGCGTATAGCCGCTATCGCATTGGCAACTATAAACTGAA |
| | CACCGATCATAGCAGCAGCAGCGATAACATTGCGCTGCTGGTGCAGCTTA |
| | AGGAGCTCATGTTTGTGTTTCTGGTGCTGCTGCCGCTGGTGAGCAGCCAG |
| | TGCGTGAACCTGACCACCCGCACCCAGCTGCCGCCGGCGTATACCAACAG |
| | CTTTACCCGCGGCGTGTATTATCCGGATAAAGTGTTTCGCAGCAGCGTGC |
| | TGCATAGCACCCAGGATCTGTTTCTGCCGTTTTTTAGCAACGTGACCTGG |
| | TTTCATGCGATTCATGTGAGCGGCACCAACGGCACCAAACGCTTTGATAA |
| | CCCGGTGCTGCCGTTTAACGATGGCGTGTATTTTGCGAGCACCGAAAAAA |
| | GCAACATTATTCGCGGCTGGATTTTTGGCACCACCCTGGATAGCAAAACC |
| | CAGAGCCTGCTGATTGTGAACAACGCGACCAACGTGGTGATTAAAGTGTG |
| | CGAATTTCAGTTTTGCAACGATCCGTTTCTGGGCGTGTATTATCATAAAA |
| | ACAACAAAAGCTGGATGGAAAGCGAATTTCGCGTGTATAGCAGCGCGAAC |
| | AACTGCACCTTTGAATATGTGAGCCAGCCGTTTCTGATGGATCTGGAAGG |
| | CAAACAGGGCAACTTTAAAAACCTGCGCGAATTTGTGTTTAAAAACATTG |
| | ATGGCTATTTTAAAATTTATAGCAAACATACCCCGATTAACCTGGTGCGC |
| | GATCTGCCGCAGGGCTTTAGCGCGCTGGAACCGCTGGTGGATCTGCCGAT |
| | TGGCATTAACATTACCCGCTTTCAGACCCTGCTGGCGCTGCATCGCAGCT |
| | ATCTGACCCCGGGCGATAGCAGCAGCGGCTGGACCGCGGGCGCGGCGGCG |
| | TATTATGTGGGCTATCTGCAGCCGCGCACCTTTCTGCTGAAATATAACGA |
| | AAACGGCACCATTACCGATGCGGTGGATTGCGCGCTGGATCCGCTGAGCG |
| | AAACCAAATGCACCCTGAAAAGCTTTACCGTGGAAAAAGGCATTTATCAG |
| | ACCAGCAACTTTCGCGTGCAGCCGACCGAAAGCATTGTGCGCTTTCCGAA |
| | CATTACCAACCTGTGCCCGTTTGGCGAAGTGTTTAACGCGACCCGCTTTG |
| | CGAGCGTGTATGCGTGGAACCGCAAACGCATTAGCAACTGCGTGGCGGAT |
| | TATAGCGTGCTGTATAACAGCGCGAGCTTTAGCACCTTTAAATGCTATGG |
| | CGTGAGCCCGACCAAACTGAACGATCTGTGCTTTACCAACGTGTATGCGG |
| | ATAGCTTTGTGATTCGCGGCGATGAAGTGCGCCAGATTGCGCCGGGCCAG |
| | ACCGGCAAAATTGCGGATTATAACTATAAACTGCCGGATGATTTTACCGG |
| | CTGCGTGATTGCGTGGAACAGCAACAACCTGGATAGCAAAGTGGGCGGCA |
| | ACTATAACTATCTGTATCGCCTGTTTCGCAAAAGCAACCTGAAACCGTTT |
| | GAACGCGATATTAGCACCGAAATTTATCAGGCGGGCAGCACCCCGTGCAA |
| | CGGCGTGGAAGGCTTTAACTGCTATTTTCCGCTGCAGAGCTATGGCTTTC |
| | AGCCGACCAACGGCGTGGGCTATCAGCCGTATCGCGTGGTGGTGCTGAGC |
| | TTTGAACTGCTGCATGCGCCGGCGACCGTGTGCGGCCCGAAAAAAAGCAC |
| | CAACCTGGTGAAAAACAAATGCGTGAACTTTAACTTTAACGGCCTGACCG |
| | GCACCGGCGTGCTGACCGAAAGCAACAAAAAATTTCTGCCGTTTCAGCAG |
| | TTTGGCCGCGATATTGCGGATACCACCGATGCGGTGCGCGATCCGCAGAC |
| | CCTGGAAATTCTGGATATTACCCCGTGCAGCTTTGGCGGCGTGAGCGTGA |
| | TTACCCCGGGCACCAACACCAGCAACCAGGTGGCGGTGCTGTATCAGGGC |
| | GTGAACTGCACCGAAGTGCCGGTGGCGATTCATGCGGATCAGCTGACCCC |
| | GACCTGGCGCGTGTATAGCACCGGCAGCAACGTGTTTCAGACCCGCGCGG |
| | GCTGCCTGATTGGCGCGGAACATGTGAACAACAGCTATGAATGCGATATT |
| | CCGATTGGCGCGGGCATTTGCGCGAGCTATCAGACCCAGACCAACAGCCC |
| | GGGCAGCGCGAGCAGCGTGGCGAGCCAGAGCATTATTGCGTATACCATGA |
| | GCCTGGGCGCGGAAAACAGCGTGGCGTATAGCAACAACAGCATTGCGATT |
| | CCGACCAACTTTACCATTAGCGTGACCACCGAAATTCTGCCGGTGAGCAT |
| | GACCAAAACCAGCGTGGATTGCACCATGTATATTTGCGGCGATAGCACCG |
| | AATGCAGCAACCTGCTGCTGCAGTATGGCAGCTTTTGCACCCAGCTGAAC |
| | CGCGCGCTGACCGGCATTGCGGTGGAACAGGATAAAAACACCCAGGAAGT |
| | GTTTGCGCAGGTGAAACAGATTTATAAAACCCCGCCGATTAAAGATTTTG |
| | GCGGCTTTAACTTTAGCCAGATTCTGCCGGATCCGAGCAAACCGAGCAAA |
| | CGCAGCTTTATTGAAGATCTGCTGTTTAACAAAGTGACCCTGGCGGATGC |
| | GGGCTTTATTAAACAGTATGGCGATTGCCTGGGCGATATTGCGGCGCGCG |
| | ATCTGATTTGCGCGCAGAAATTTAACGGCCTGACCGTGCTGCCGCCGCTG |
| | CTGACCGATGAAATGATTGCGCAGTATACCAGCGCGCTGCTGGCGGGCAC |
| | CATTACCAGCGGCTGGACCTTTGGCGCGGGCGCGGCGCTGCAGATTCCGT |
| | TTGCGATGCAGATGGCGTATCGCTTTAACGGCATTGGCGTGACCCAGAAC |
| | GTGCTGTATGAAAACCAGAAACTGATTGCGAACCAGTTTAACAGCGCGAT |
| | TGGCAAAATTCAGGATAGCCTGAGCAGCACCGCGAGCGCGCTGGGCAAAC |
| | TGCAGGATGTGGTGAACCAGAACGCGCAGGCGCTGAACACCCTGGTGAAA |
| | CAGCTGAGCAGCAACTTTGGCGCGATTAGCAGCGTGCTGAACGATATTCT |
| | GAGCCGCCTGGATCCGCCGGAAGCGGAAGTGCAGATTGATCGCCTGATTA |
| | CCGGCCGCCTGCAGAGCCTGCAGACCTATGTGACCCAGCAGCTGATTCGC |
| | GCGGCGGAAATTCGCGCGAGCGCGAACCTGGCGGCGACCAAAATGAGCGA |
| | ATGCGTGCTGGGCCAGAGCAAACGCGTGGATTTTTGCGGCAAAGGCTATC |
| | ATCTGATGAGCTTTCCGCAGAGCGCGCCGCATGGCGTGGTGTTTCTGCAT |
| | GTGACCTATGTGCCGGCGCAGGAAAAAAACTTTACCACCGCGCCGGCGAT |
| | TTGCCATGATGGCAAAGCGCATTTTCCGCGCGAAGGCGTGTTTGTGAGCA |
| | ACGGCACCCATTGGTTTGTGACCCAGCGCAACTTTTATGAACCGCAGATT |
| | ATTACCACCGATAACACCTTTGTGAGCGGCAACTGCGATGTGGTGATTGG |
| | CATTGTGAACAACACCGTGTATGATCCGCTGCAGCCGGAACTGGATAGCT |
| | TTAAAGAAGAACTGGATAAATATTTTAAAAACCATACCAGCCCGGATGTG |
| | GATCTGGGCGATATTAGCGGCATTAACGCGAGCGTGGTGAACATTCAGAA |
| | AGAAATTGATCGCCTGAACGAAGTGGCGAAAAACCTGAACGAAAGCCTGA |
| | TTGATCTGCAGGAACTGGGCAAATATGAACAGGGCTATATTCCGGAAGCG |
| | CCGCGCGATGGCCAGGCGTATGTGCGCAAAGATGGCGAATGGGTGCTGCT |
| | GAGCACCTTTCTGGCATTAGCCTATGGAAGTCAGGGTAATCTTAATCCAT |
| | TAATTAATGAAATCAGCAAAATCATTTCAGCTGCAGGTAATTTTGATGTT |
| | AAAGAGGAAAGAGCGGCCGCGTCTTTATTGCAGTTGTCCGGTAATGCCAG |
| | TGATTTTTCATATGGACGGAACTCAATAACTTTGACAGCATCAGCATAAG |
| | AGCTCtttattaatttaaataatagcaatcttactgggctgtgccacata |
| | agattgctatttttttggagtcataatggattcttgtcataaaattgatt |
| | atgggttatacgccctggagattttagcccaataccataacgtctctgtt |
| | aacccggaagaaattaaacatagatttgacacagacgggactggtctggg |
| | attaacgtcatggttgcttgctgcgaaatctttagaactaaaggtaaaac |
| | aggtaaaaaaaacaattgaccgattaaactttatttctctgcccgcatta |
| | gtctggagagaggatggatgtcattttattctgactaaagtcagtaaaga |
| | agcaaacagatatcttatttttgatctggagcagcgaaatccccgtgttc |
| | tcgaacagtctgagtttgaggcgttatatcaggggcatattattcttatt |
| | gcttcccgttcttctgttaccgggaaactggcaaaatttgactttacctg |
| | gtttattcctgccattataaaatacaggaaaatatttattgaaacccttg |
| | ttgtatctgtttttttacaattatttgcattaataacccccctttttttt |
| | caggtggttatggacaaagtattagtacacagggggttttcaacccttaa |
| | tgttattactgtcgcattatctgttgtggtggtgtttgagattatactca |
| | gcggtttaagaacttacatttttgcacatagtacaagtcggattgatgtt |
| | gagttgggtgccaaactcttccggcatttactggcgctaccgatctctta |
| | ttttgagagtcgtcgtgttggtgatactgttgccagggtaagagaattag |
| | accagatccgtaatttcctgacaggacaggcattaacatctgttctggac |
| | ttattattttcattcatattttttgcggtaatgtggtattacagcccaaa |
| | gcttactctggtgatcttattttcgctgccctgttatgctgcatggtctg |
| | tttttattagccccattttgcgacgtcgccttgatgataagttttcacgg |
| | aatgcggataatcaatctttcctggtggaatcagtcacggcgattaacac |
| | tataaaagctatggcagtctcacctcagatgacgaacatatgggacaaac |
| | aattggcaggatatgttgctgcaggctttaaagtgacagtattagccacc |
| | attggtcaacaaggaatacagttaatacaaaagactgttatgatcatcaa |
| | cctgtggttgggtgcacacctggttatttccggggatttaagtattggtc |
| | agttaattgcttttaatatgcttgcaggtcagattgttgcaccggttatt |
| | cgccttgcacaaatctggcaggatttccagcaggttggtatatcagttac |
| | ccgccttggtgatgtgcttaactctccaactgaaagttatcatgggaaac |
| | tggcattaccggaaattaatggtgatatcacttttcgtaatatccggttt |
| | cgctataagcctgactctccggttattttagataatatcaatctcagtat |
| | taagcagggggaggttattggtattgtcggacgttctggttcaggaaaaa |
| | gcacattaactaaattaattcaacgtttttatattcctgaaaatggccag |
| | gtcttaattgatggacatgatcttgcgttggccgatcctaactggttacg |
| | tcgtcaggtgggggttgtgttgcaggacaatgtgctgcttaatcgcagta |
| | ttattgataatatttcactggctaatcctggcatgtccgtcgaaaaagtt |
| | atttatgcagcgaaattagcaggtgctcatgattttatttctgaattgcg |
| | tgaggggtataacaccattgtcggggaacagggggcaggattatccggag |
| | gtcaacgtcaacgcatcgcaattgcaagggcgctggtgaacaaccctaaa |
| | atactcatctttgatgaagcaaccagtgctctggattatgagtcggagca |
| | tgtcatcatgcgcaatatgcacaaaatatgtaagggcagaacggttataa |
| | tcattgctcatcgtctgtctacagtaaaaaatgcagaccgcattattgtc |
| | atggaaaaagggaaaattgttgaacagggtaaacataaggagctgctttc |
| | tgaaccggaaagtttatacagttacttatatcagttacagtcagactaac |
| | agaaagaacagaagaatatgaaaacatggttaatggggttcagcgagttc |
| | ctgttgcgctataaacttgtctggagtgaaacatggaaaatccggaagca |
| | gttagatactccggtacgtgaaaaggacgaaaatgaattcttacccgctc |
| | atctggaattaattgaaacgccggtatccagacggccgcgtctggttgct |
| | tattttattatggggtttctggttattgctttcattttatctgttttagg |
| | ccaggtggaaattgttgccactgcaaatgggaaattaacactcagtgggc |
| | gtagcaaagaaattaaacctattgaaaactcgatagttaaagaaattatc |
| | gtaaaagaaggagagtcagtccggaaaggggatgtgttattaaagcttac |
| | agcgctgggagctgaagctgatacgttaaaaacgcagtcatcactgttac |
| | aggccaggctggaacaaattcggtatcaaattctgagccggtcaattgaa |
| | ttaaataaacttcctgaactgaaacttcctgatgagccttattttcagaa |
| | tgtatctgaagaggaagtactgcgtttaacttctttgataaaagaacagt |
| | tttccacatggcaaaatcagaagtatcaaaaagaactgaatctggataag |
| | aaaagagcagagcgattaacaatacttgcccgtataaaccgttatgaaaa |
| | tgtatcgagggttgaaaaaagccgtctggatgatttcaggagcctgttgc |
| | ataaacaggcaattgcaaaacatgctgtacttgagcaggagaataaatat |
| | gttgaggcagcaaatgaattacgggtttataaatcgcaactggagcaaat |
| | tgagagtgagatattgtctgcaaaagaagaatatcagcttgtcacgcagc |
| | tttttaaaaatgaaattttagacaagctaagacaaacaacagacagcatt |
| | gagttattaactctggagttagagaaaaatgaagagcgtcaacaggcttc |
| | agtaatcagggcccctgtttcgggaaaagttcagcaactgaaggttcata |
| | ctgaaggtggggttgttacaacagcggaaacactgatggtcatcgttccg |
| | gaagatgacacgctggaggttactgctctggtacaaaataaagatattgg |
| | ttttattaacgtcgggcagaatgccatcattaaagtggaggcttttcctt |
| | acacccgatatggttatctggtgggtaaggtaaaaaatataaatttagat |
| | gcaatagaagaccagaaactgggactcgtttttaatgtcattgtttctgt |
| | tgaagagaatgatttgtcaaccgggaataagcacattccattaagctcgg |
| | gtatggctgtcactgcagaaataaagactggaatgcgaagcgtaatcagt |
| | tatcttcttagtcccctggaagaatctgtaacagaaagtttacatgagcg |
| | ttaagtctcagagcagcggtatccggctcatatcttctcctgtcagatcc |
| | aagggcgaattccagcacactggcggccgttactagtggatccaccggtc |
| | tcgaggggcgcgccggtaccgaattctaattaatgggcatcatttttcag |
| | ctatttcatttataaaataagttatgaaaaaaatcatcatattactaacg |
| | acatttttcctgctttcgggatgcactattcccagggcggtatttaaatc |
| | cagccttattaatcaggacgatcctcgttataatctggtcgaagtcacgc |
| | cgacattaaaactaagcgctcccgatactgtgccgaaaactattgtcgat |
| | ccggtttttgccgcaaataactggcactggacatctttggctaaaggcga |
| | tgtgctgcatatcactattttatcctcgggcggggctggatatttatcca |
| | ataacgcgagcggcgaccgtgcggattttgaaaatattcttgtgactgac |
| | agtaataccgttcaggtgccttatgccgggacaatcccggtttctggatt |
| | ggatgtgacgcaactggctgatgagatcaaaaagcgactttcgcgcgttg |
| | tcctgaatcctcaggtgattgtgacacttaccgcccgcaccggagccatg |
| | gtgacggtcgagggcagcgggaaaacgggtcgataccctctcgaacagag |
| | tatgaatcgtctgagtcatcttttggcaacggcagtggcagtagaaaata |
| | ccagcacagatatgatggaagttcacgtgacgcgccagcagcattatttc |
| | actgcgcgactgtcagatatttatcagtatcccggattggatattgcctt |
| | acagccggatgaccgtatcactctgcgtcaagtgaccgagtatgttaatg |
| | tgctcggcgcagcaggtgttcaggggaaacatgctctggttcagcgtcat |
| | tccagcgtcgtggatgctttggcgctcgcgaaaggattaaatgacaatct |
| | tgccgatccgcaagcgatttttctgtacaagcataacgaagcggaacagg |
| | cgaaacagcagatgcgtaagctgaatatctatcatgtcgatatgagccaa |
| | cctaactcggtgtttttagcgcaggccatacgagtggacaacggcgatgt |
| | tatctatatctcgaacgcctctttgactgatttcgctaaggtgaaagccg |
| | cattcgatagctttttgac |
| SEQ ID NO: 11 (SARS- | RVQPTESIVRFPNITNLCPFGEVFNATRFASVYAWNRKRISNCVADYSVL |
| CoV-2 RBD) | YNSASFSTFKCYGVSPTKLNDLCFTNVYADSFVIRGDEVRQIAPGQTGKI |
| | ADYNYKLPDDFTGCVIAWNSNNLDSKVGGNYNYLYRLFRKSNLKPFERDI |
| | STEIYQAGSTPCNGVEGFNCYFPLQSYGFQPTNGVGYQPYRVVVLSFELL |
| | HAPATVCGPKKSTNLVKNKCVNF |
| SEQ ID NO: 12 (14 | GYIPEAPRDGQAYVRKDGEWVLLSTFL |
| Fibritin Trimerization | |
| Motif) | |
| SEQ ID NO: 13 (HLYA | ALAYGSQGNLNPLINEISKIISAAGNFDVKEERAAASLLQLSGNASDFSY |
| signal sequence) | GRNSITLTASA |
| SEQ ID NO: 14 (YbaS) | MLDANKLQQAVDQAYTQFHSLNGGQNADYIPFLANVPGQLAAVAIVTCDG |
| | NVYSAGDSDYRFALESISKVCTLALALEDVGPQAVQDKIGADPTGLPFNS |
| | VIALELHGGKPLSPLVNAGAIATTSLINAENVEQRWQRILHIQQQLAGEQ |
| | VALSDEVNQSEQTTNFHNRAIAWLLYSAGYLYCDAMEACDVYTRQCSTLI |
| | NTVELATLGATLAAGGVNPLTHKRVLQADNVPYILAEMMMEGLYGRSGDW |
| | AYRVGLPGKSGVGGGILAVVPGVMGIAAFSPPLDEEGNSVRGQKMVASVA |
| | KQLGYNVFKG |
| SEQ ID NO: 15 (YbaS) | ATGTTAGATGCAAACAAATTACAGCAGGCAGTGGATCAGGCTTACACCCA |
| | ATTTCACTCACTTAACGGCGGACAAAATGCCGATTACATTCCCTTTCTGG |
| | CGAATGTACCAGGTCAACTGGCGGCAGTGGCTATCGTGACNTGCGATGGC |
| | AACGTCTATAGTGCGGGTGACAGTGATTACCGCTTTGCACTGGAATCCAT |
| | CTCTAAAGTCTGTACGTTAGCCCTTGCGTTAGAAGATGTCGGCCCGCAGG |
| | CGGTACAGGACAAAATTGGCGCTGACCCGACCGGATTGCCCTTTAACTCA |
| | GTTATCGCCTTAGAGTTGCATGGCGGCAAACCGCTGTCGCCACTGGTAAA |
| | TGCTGGCGCTATTGCCACCACCAGCCTGATTAACGCTGAAAATGTTGAAC |
| | AACGCTGGCAGCGAATTTTACATATCCAACAGCAACTGGCTGGTGAGCAG |
| | GTAGCGCTCTCTGACGAAGTCAACCAGTCGGAACAAACAACCAACTTCCA |
| | TAACCGGGCCATTGCCTGGCTGCTGTACTCCGCCGGATATCTCTATTGTG |
| | ATGCAATGGAAGCCTGTGACGTGTACACCCGTCAGTGCTCTACGCTCATC |
| | AATACTGTTGAACTGGCAACGCTTGGCGCGACGCTGGCGGCGGGTGGTGT |
| | GAATCCGTTGACGCATAAACGCGTTCTTCAGGCCGACAACGTGCCGTACA |
| | TTCTGGCCGAAATGATGATGGAAGGGCTGTATGGTCGCTCCGGTGACTGG |
| | GCGTATCGCGTTGGTTTACCGGGCAAAAGCGGTGTAGGTGGCGGTATTCT |
| | GGCGGTCGTCCCTGGCGTGATGGGAATTGCCGCGTTCTCACCACCGCTGG |
| | ACGAAGAAGGCAACAGTGTTCGCGGTCAAAAAATGGTGGCATCGGTCGCT |
| | AAGCAACTCGGCTATAACGTGTTTAAGGGCTGA |
| SEQ ID NO: 16 (GadB) | MRSNHFKEFKMDKKQVTDLRSELLDSRFGAKSISTIAESKRFPLHEMRDD |
| | VAFQIINDELYLDGNARQNLATFCQTWDDENVHKLMDLSINKNWIDKEEY |
| | PQSAAIDLRCVNMVADLWHAPAPKNGQAVGTNTIGSSEACMLGGMAMKWR |
| | WRKRMEAAGKPTDKPNLVCGPVQICWHKFARYWDVELREIPMRPGQLFMD |
| | PKRMIEACDENTIGVVPTFGVTYTGNYEFPQPLHDALDKFQADTGIDIDM |
| | HIDAASGGFLAPFVAPDIVWDFRLPRVKSISASGHKFGLAPLGCGWVIWR |
| | DEEALPQELVFNVDYLGGQIGTFAINFSRPAGQVIAQYYEFLRLGREGYT |
| | KVQNASYQVAAYLADEIAKLGPYEFICTGRPDEGIPAVCFKLKDGEDPGY |
| | TLYDLSERLRLRGWQVPAFTLGGEATDIVVMRIMCRRGFEMDFAELLLED |
| | YKASLKYLSDHPKLQGIAQQNSFKHT |
| SEQ ID NO: 17 (GadB) | ATGCGATCCAATCATTTTAAGGAGTTTAAAATGGATAAGAAGCAAGTAAC |
| | GGATTTAAGGTCGGAACTACTCGATTCACGTTTTGGTGCGAAGTCTATTT |
| | CCACTATCGCAGAATCAAAACGTTTTCCGCTGCACGAAATGCGCGACGAT |
| | GTCGCATTTCAGATTATCAATGATGAATTATATCTTGATGGCAACGCTCG |
| | TCAGAACCTGGCCACTTTCTGCCAGACCTGGGACGACGAAAACGTCCACA |
| | AGTTGATGGATTTATCCATTAACAAAAACTGGATCGACAAAGAAGAATAT |
| | CCGCAATCCGCAGCCATCGACCTGCGTTGCGTAAACATGGTTGCCGATCT |
| | GTGGCATGCGCCTGCGCCGAAAAATGGTCAGGCCGTTGGCACCAACACCA |
| | TTGGTTCTTCCGAGGCCTGTATGCTCGGCGGGATGGCGATGAAATGGCGT |
| | TGGCGCAAGCGTATGGAAGCTGCAGGCAAACCAACGGATAAACCAAACCT |
| | GGTGTGCGGTCCGGTGCAAATCTGCTGGCATAAATTCGCCCGCTACTGGG |
| | ATGTGGAGCTGCGTGAGATCCCTATGCGCCCCGGTCAGTTGTTTATGGAC |
| | CCGAAACGCATGATTGAAGCCTGCGACGAAAATACCATCGGCGTGGTGCC |
| | GACTTTCGGCGTGACCTACACCGGTAACTATGAGTTCCCGCAACCGCTGC |
| | ACGATGCACTGGATAAATTCCAGGCCGACACCGGTATCGACATCGACATG |
| | CACATCGACGCCGCCAGCGGTGGCTTTCTGGCACCGTTCGTCGCCCCGGA |
| | TATCGTCTGGGACTTCCGCCTGCCGCGTGTGAAATCGATCAGTGCTTCAG |
| | GCCATAAATTCGGTCTGGCTCCGCTGGGCTGCGGCTGGGTTATCTGGCGT |
| | GACGAAGAAGCGCTGCCGCAGGAACTGGTGTTCAACGTTGACTACCTCGG |
| | TGGTCAGATTGGTACTTTTGCCATCAACTTCTCCCGCCCGGCGGGTCAGG |
| | TGATTGCACAGTACTATGAATTCCTGCGCCTTGGTCGTGAAGGCTATACC |
| | AAAGTACAGAATGCTTCCTACCAGGTCGCTGCCTATCTGGCGGATGAGAT |
| | CGCCAAACTGGGGCCGTATGAGTTCATCTGTACCGGTCGCCCGGACGAAG |
| | GCATCCCGGCGGTTTGCTTCAAACTGAAAGATGGTGAAGATCCGGGATAC |
| | ACCCTGTACGACCTCTCTGAACGTCTGCGTCTGCGCGGCTGGCAGGTTCC |
| | GGCCTTCACTCTCGGCGGTGAAGCCACCGACATCGTGGTGATGCGCATTA |
| | TGTGTCGTCGCGGCTTCGAAATGGACTTTGCTGAACTGTTGCTGGAAGAC |
| | TACAAAGCCTCCCTGAAATATCTCAGCGATCACCCGAAACTGCAGGGTAT |
| | TGCCCAGCAGAACAGCTTTAAACATACCTGA |
| SEQ ID NO: 18 (GadC) | MATSVQTGKAKQLTLLGFFAITASMVMAVYEYPTFATSGFSLVFFLLLGG |
| | ILWFIPVGLCAAEMATVDGWEEGGVFAWVSNTLGPRWGFAAISFGYLQIA |
| | IGFIPMLYFVLGALSYILKWPALNEDPITKTIAALIILWALALTQFGGTK |
| | YTARIAKVGFFAGILLPAFILIALAAIYLHSGAPVAIEMDSKTFFPDFSK |
| | VGTLVVFVAFILSYMGVEASATHVNEMSNPGRDYPLAMLLLMVAAICLSS |
| | VGGLSIAMVIPGNEINLSAGVMQTFTVLMSHVAPEIEWTVRVISALLLLG |
| | VLAEIASWIVGPSRGMYVTAQKNLLPAAFAKMNKNGVPVTLVISQLVITS |
| | IALIILTNTGGGNNMSFLIALALTVVIYLCAYFMLFIGYIVLVLKHPDLK |
| | RTFNIPGGKGVKLVVAIVGLLTSIMAFIVSFLPPDNIQGDSTDMYVELLV |
| | VSFLVVLALPFILYAVHDRKGKANTGVTLEPINSQNAPKGHFFLHPRARS |
| | PHYIVMNDKKH |
| SEQ ID NO: 19 (GadC) | ATGGCTACATCAGTACAGACAGGTAAAGCTAAGCAGCTCACATTACTCGG |
| | ATTCTTTGCCATAACGGCATCGATGGTAATGGCTGTTTATGAATACCCTA |
| | CCTTCGCAACATCGGGCTTTTCATTAGTCTTCTTCCTGCTATTAGGCGGG |
| | ATTTTATGGTTTATTCCCGTGGGACTTTGTGCTGCGGAAATGGCCACCGT |
| | CGACGGCTGGGAAGAAGGTGGTGTCTTCGCCTGGGTATCAAATACTCTGG |
| | GTCCGAGATGGGGATTTGCAGCGATCTCATTTGGCTATCTGCAAATCGCC |
| | ATTGGTTTTATTCCGATGCTCTATTTCGTGTTAGGGGCACTCTCCTACAT |
| | CCTGAAATGGCCAGCGCTGAATGAAGACCCCATTACCAAAACTATTGCAG |
| | CACTCATCATTCTTTGGGCGCTGGCATTAACGCAGTTTGGTGGCACGAAA |
| | TACACGGCGCGAATTGCTAAAGTTGGCTTCTTCGCCGGTATCCTGTTACC |
| | TGCATTTATTTTGATCGCATTAGCGGCTATTTACCTGCACTCCGGTGCCC |
| | CCGTTGCTATCGAAATGGATTCGAAGACCTTCTTCCCTGACTTCTCTAAA |
| | GTGGGCACACTGGTTGTATTTGTTGCCTTCATTTTGAGTTATATGGGCGT |
| | AGAAGCTTCCGCAACTCACGTCAATGAAATGAGCAACCCCGGGCGCGACT |
| | ATCCACTGGCTATGTTACTGCTGATGGTGGCGGCAATCTGCTTAAGCTCT |
| | GTTGGCGGTTTGTCTATTGCGATGGTCATTCCGGGTAATGAAATCAACCT |
| | CTCCGCAGGGGTAATGCAAACCTTTACCGTTCTGATGTCCCATGTGGCAC |
| | CGGAAATTGAGTGGACGGTTCGCGTGATCTCCGCACTGCTGTTGCTGGGT |
| | GTTCTGGCGGAAATCGCCTCCTGGATTGTTGGTCCTTCTCGCGGGATGTA |
| | TGTCACAGCGCAGAAAAACCTGCTGCCAGCGGCATTCGCTAAAATGAACA |
| | AAAATGGCGTACCGGTAACGCTGGTCATTTCGCAGCTGGTGATTACTTCT |
| | ATCGCGTTGATCATCCTCACCAATACCGGTGGCGGTAACAACATGTCCTT |
| | CCTGATCGCACTGGCGCTGACGGTGGTGATTTATCTGTGTGCTTATTTCA |
| | TGCTGTTTATTGGCTACATTGTGTTGGTTCTTAAACATCCTGACTTAAAA |
| | CGCACATTTAATATCCCTGGTGGTAAAGGGGTGAAACTGGTCGTGGCAAT |
| | TGTCGGTCTGCTGACTTCAATTATGGCGTTTATTGTTTCCTTCCTGCCGC |
| | CGGATAACATCCAGGGTGATTCTACCGATATGTATGTTGAATTACTGGTT |
| | GTTAGTTTCCTGGTGGTACTTGCCCTGCCCTTTATTCTCTATGCTGTTCA |
| | TGATCGTAAAGGCAAAGCGAATACCGGCGTCACTCTGGAGCCAATCAACA |
| | GTCAGAACGCACCAAAAGGTCACTTCTTCCTGCACCCGCGTGCACGTTCA |
| | CCACACTATATTGTGATGAATGACAAGAAACACTAA |
| SEQ ID NO: 20 (araC- | CGATCGTTATGACAACTTGACGGCTACATCATTCACTTTTTCTTCACAAC |
| YbaS-GadBC AR | CGGCACGGAACTCGCTCGGGCTGGCCCCGGTGCATTTTTTAAATACCCGC |
| cassette) | GAGAAGTAGAGTTGATCGTCAAAACCAACATTGCGACCGACGGTGGCGAT |
| | AGGCATCCGGGTGGTGCTCAAAAGCAGCTTCGCCTGGCTGATACGTTGGT |
| | CCTCGCGCCAGCTTAAGACGCTAATCCCTAACTGCTGGCGGAAAAGATGT |
| | GACAGACGCGACGGCGACAAGCAAACATGCTGTGCGACGCTGGCGATATC |
| | AAAATTGCTGTCTGCCAGGTGATCGCTGATGTACTGACAAGCCTCGCGTA |
| | CCCGATTATCCATCGGTGGATGGAGCGACTCGTTAATCGCTTCCATGCGC |
| | CGCAGTAACAATTGCTCAAGCAGATTTATCGCCAGCAGCTCCGAATAGCG |
| | CCCTTCCCCTTGCCCGGCGTTAATGATTTGCCCAAACAGGTCGCTGAAAT |
| | GCGGCTGGTGCGCTTCATCCGGGCGAAAGAACCCCGTATTGGCAAATATT |
| | GACGGCCAGTTAAGCCATTCATGCCAGTAGGCGCGCGGACGAAAGTAAAC |
| | CCACTGGTGATACCATTCGCGAGCCTCCGGATGACGACCGTAGTGATGAA |
| | TCTCTCCTGGCGGGAACAGCAAAATAACACCCGGTCGGCAAACAAATTCT |
| | CGTCCCTGATTTTTCACCACCCCCTGACCGCGAATGGTGAGATTGAGAAT |
| | ATAACCTTTCATTCCCAGCGGTCGGTCGATAAAAAAATCGAGATAACCGT |
| | TGGCCTCAATCGGCGTTAAACCCGCCACCAGATGGGCATTAAACGAGTAT |
| | CCCGGCAGCAGGGGATCATTTTGCGCTTCAGCCATACTTTTCATACTCCC |
| | GCCATTCAGAGAAGAAACCAATTGTCCATATTGCATCAGACATTGCCGTC |
| | ACTGCGTCTTTTACTGGCTCTTCTCGCTAACCAAACCGGTAACCCCGCTT |
| | ATTAAAAGCATTCTGTAACAAAGCGGGACCAAAGCCATGACAAAAACGCG |
| | TAACAAAAGTGTCTATAATCACGGCAGAAAAGTCCACATTGATTATTTGC |
| | ACGGCGTCACACTTTGCTATGCCATAGCATTTTTATCCATAAGATTAGCG |
| | GATCCTACCTGACGCTTTTTATCGCAACTCTCTACTGTTTCTCCATACCC |
| | GTTTTTTTGGGAATTCGAGCTCGGAGTTAACAAAAGATGTTAGATGCAAA |
| | CAAATTACAGCAGGCAGTGGATCAGGCTTACACCCAATTTCACTCACTTA |
| | ACGGCGGACAAAATGCCGATTACATTCCCTTTCTGGCGAATGTACCAGGT |
| | CAACTGGCGGCAGTGGCTATCGTGACNTGCGATGGCAACGTCTATAGTGC |
| | GGGTGACAGTGATTACCGCTTTGCACTGGAATCCATCTCTAAAGTCTGTA |
| | CGTTAGCCCTTGCGTTAGAAGATGTCGGCCCGCAGGCGGTACAGGACAAA |
| | ATTGGCGCTGACCCGACCGGATTGCCCTTTAACTCAGTTATCGCCTTAGA |
| | GTTGCATGGCGGCAAACCGCTGTCGCCACTGGTAAATGCTGGCGCTATTG |
| | CCACCACCAGCCTGATTAACGCTGAAAATGTTGAACAACGCTGGCAGCGA |
| | ATTTTACATATCCAACAGCAACTGGCTGGTGAGCAGGTAGCGCTCTCTGA |
| | CGAAGTCAACCAGTCGGAACAAACAACCAACTTCCATAACCGGGCCATTG |
| | CCTGGCTGCTGTACTCCGCCGGATATCTCTATTGTGATGCAATGGAAGCC |
| | TGTGACGTGTACACCCGTCAGTGCTCTACGCTCATCAATACTGTTGAACT |
| | GGCAACGCTTGGCGCGACGCTGGCGGCGGGTGGTGTGAATCCGTTGACGC |
| | ATAAACGCGTTCTTCAGGCCGACAACGTGCCGTACATTCTGGCCGAAATG |
| | ATGATGGAAGGGCTGTATGGTCGCTCCGGTGACTGGGCGTATCGCGTTGG |
| | TTTACCGGGCAAAAGCGGTGTAGGTGGCGGTATTCTGGCGGTCGTCCCTG |
| | GCGTGATGGGAATTGCCGCGTTCTCACCACCGCTGGACGAAGAAGGCAAC |
| | AGTGTTCGCGGTCAAAAAATGGTGGCATCGGTCGCTAAGCAACTCGGCTA |
| | TAACGTGTTTAAGGGCTGACCATGGAGTGTTTTAATGCGATCCAATCATT |
| | TTAAGGAGTTTAAAATGGATAAGAAGCAAGTAACGGATTTAAGGTCGGAA |
| | CTACTCGATTCACGTTTTGGTGCGAAGTCTATTTCCACTATCGCAGAATC |
| | AAAACGTTTTCCGCTGCACGAAATGCGCGACGATGTCGCATTTCAGATTA |
| | TCAATGATGAATTATATCTTGATGGCAACGCTCGTCAGAACCTGGCCACT |
| | TTCTGCCAGACCTGGGACGACGAAAACGTCCACAAGTTGATGGATTTATC |
| | CATTAACAAAAACTGGATCGACAAAGAAGAATATCCGCAATCCGCAGCCA |
| | TCGACCTGCGTTGCGTAAACATGGTTGCCGATCTGTGGCATGCGCCTGCG |
| | CCGAAAAATGGTCAGGCCGTTGGCACCAACACCATTGGTTCTTCCGAGGC |
| | CTGTATGCTCGGCGGGATGGCGATGAAATGGCGTTGGCGCAAGCGTATGG |
| | AAGCTGCAGGCAAACCAACGGATAAACCAAACCTGGTGTGCGGTCCGGTG |
| | CAAATCTGCTGGCATAAATTCGCCCGCTACTGGGATGTGGAGCTGCGTGA |
| | GATCCCTATGCGCCCCGGTCAGTTGTTTATGGACCCGAAACGCATGATTG |
| | AAGCCTGCGACGAAAATACCATCGGCGTGGTGCCGACTTTCGGCGTGACC |
| | TACACCGGTAACTATGAGTTCCCGCAACCGCTGCACGATGCACTGGATAA |
| | ATTCCAGGCCGACACCGGTATCGACATCGACATGCACATCGACGCCGCCA |
| | GCGGTGGCTTTCTGGCACCGTTCGTCGCCCCGGATATCGTCTGGGACTTC |
| | CGCCTGCCGCGTGTGAAATCGATCAGTGCTTCAGGCCATAAATTCGGTCT |
| | GGCTCCGCTGGGCTGCGGCTGGGTTATCTGGCGTGACGAAGAAGCGCTGC |
| | CGCAGGAACTGGTGTTCAACGTTGACTACCTCGGTGGTCAGATTGGTACT |
| | TTTGCCATCAACTTCTCCCGCCCGGCGGGTCAGGTGATTGCACAGTACTA |
| | TGAATTCCTGCGCCTTGGTCGTGAAGGCTATACCAAAGTACAGAATGCTT |
| | CCTACCAGGTCGCTGCCTATCTGGCGGATGAGATCGCCAAACTGGGGCCG |
| | TATGAGTTCATCTGTACCGGTCGCCCGGACGAAGGCATCCCGGCGGTTTG |
| | CTTCAAACTGAAAGATGGTGAAGATCCGGGATACACCCTGTACGACCTCT |
| | CTGAACGTCTGCGTCTGCGCGGCTGGCAGGTTCCGGCCTTCACTCTCGGC |
| | GGTGAAGCCACCGACATCGTGGTGATGCGCATTATGTGTCGTCGCGGCTT |
| | CGAAATGGACTTTGCTGAACTGTTGCTGGAAGACTACAAAGCCTCCCTGA |
| | AATATCTCAGCGATCACCCGAAACTGCAGGGTATTGCCCAGCAGAACAGC |
| | TTTAAACATACCTGATAACCGTTTTGGAGTGATAATATGGCTACATCAGT |
| | ACAGACAGGTAAAGCTAAGCAGCTCACATTACTCGGATTCTTTGCCATAA |
| | CGGCATCGATGGTAATGGCTGTTTATGAATACCCTACCTTCGCAACATCG |
| | GGCTTTTCATTAGTCTTCTTCCTGCTATTAGGCGGGATTTTATGGTTTAT |
| | TCCCGTGGGACTTTGTGCTGCGGAAATGGCCACCGTCGACGGCTGGGAAG |
| | AAGGTGGTGTCTTCGCCTGGGTATCAAATACTCTGGGTCCGAGATGGGGA |
| | TTTGCAGCGATCTCATTTGGCTATCTGCAAATCGCCATTGGTTTTATTCC |
| | GATGCTCTATTTCGTGTTAGGGGCACTCTCCTACATCCTGAAATGGCCAG |
| | CGCTGAATGAAGACCCCATTACCAAAACTATTGCAGCACTCATCATTCTT |
| | TGGGCGCTGGCATTAACGCAGTTTGGTGGCACGAAATACACGGCGCGAAT |
| | TGCTAAAGTTGGCTTCTTCGCCGGTATCCTGTTACCTGCATTTATTTTGA |
| | TCGCATTAGCGGCTATTTACCTGCACTCCGGTGCCCCCGTTGCTATCGAA |
| | ATGGATTCGAAGACCTTCTTCCCTGACTTCTCTAAAGTGGGCACACTGGT |
| | TGTATTTGTTGCCTTCATTTTGAGTTATATGGGCGTAGAAGCTTCCGCAA |
| | CTCACGTCAATGAAATGAGCAACCCCGGGCGCGACTATCCACTGGCTATG |
| | TTACTGCTGATGGTGGCGGCAATCTGCTTAAGCTCTGTTGGCGGTTTGTC |
| | TATTGCGATGGTCATTCCGGGTAATGAAATCAACCTCTCCGCAGGGGTAA |
| | TGCAAACCTTTACCGTTCTGATGTCCCATGTGGCACCGGAAATTGAGTGG |
| | ACGGTTCGCGTGATCTCCGCACTGCTGTTGCTGGGTGTTCTGGCGGAAAT |
| | CGCCTCCTGGATTGTTGGTCCTTCTCGCGGGATGTATGTCACAGCGCAGA |
| | AAAACCTGCTGCCAGCGGCATTCGCTAAAATGAACAAAAATGGCGTACCG |
| | GTAACGCTGGTCATTTCGCAGCTGGTGATTACTTCTATCGCGTTGATCAT |
| | CCTCACCAATACCGGTGGCGGTAACAACATGTCCTTCCTGATCGCACTGG |
| | CGCTGACGGTGGTGATTTATCTGTGTGCTTATTTCATGCTGTTTATTGGC |
| | TACATTGTGTTGGTTCTTAAACATCCTGACTTAAAACGCACATTTAATAT |
| | CCCTGGTGGTAAAGGGGTGAAACTGGTCGTGGCAATTGTCGGTCTGCTGA |
| | CTTCAATTATGGCGTTTATTGTTTCCTTCCTGCCGCCGGATAACATCCAG |
| | GGTGATTCTACCGATATGTATGTTGAATTACTGGTTGTTAGTTTCCTGGT |
| | GGTACTTGCCCTGCCCTTTATTCTCTATGCTGTTCATGATCGTAAAGGCA |
| | AAGCGAATACCGGCGTCACTCTGGAGCCAATCAACAGTCAGAACGCACCA |
| | AAAGGTCACTTCTTCCTGCACCCGCGTGCACGTTCACCACACTATATTGT |
| | GATGAATGACAAGAAACACTAACGATCG |
| SEQ ID NO: 21 (GadA) | MDQKLLTDFRSELLDSRFGAKAISTIAESKRFPLHEMRDDVAFQIINDEL |
| | YLDGNARQNLATFCQTWDDENVHKLMDLSINKNWIDKEEYPQSAAIDLRC |
| | VNMVADLWHAPAPKNGQAVGTNTIGSSEACMLGGMAMKWRWRKRMEAAGK |
| | PTDKPNLVCGPVQICWHKFARYWDVELREIPMRPGQLFMDPKRMIEACDE |
| | NTIGVVPTFGVTYTGNYEFPQPLHDALDKFQADTGIDIDMHIDAASGGFL |
| | APFVAPDIVWDFRLPRVKSISASGHKFGLAPLGCGWVIWRDEEALPQELV |
| | FNVDYLGGQIGTFAINFSRPAGQVIAQYYEFLRLGREGYTKVQNASYQVA |
| | AYLADEIAKQGPYEFICTGRPDEGIPAVCFKLKDGEDPGYTLYDLSERLR |
| | LRGWQVPAFTLGGEATDIVVMRIMCRRGFEMDFAELLLEDYKASLKYLSD |
| | HPKLQGIAQQNSFKHT |
| SEQ ID NO: 22 (GadA) | ATGGACCAGAAGCTGTTAACGGATTTCCGCTCAGAACTACTCGATTCACG |
| | TTTTGGCGCAAAGGCCATTTCTACTATCGCGGAGTCAAAACGATTTCCGC |
| | TGCACGAAATGCGCGATGATGTCGCATTCCAGATTATCAATGATGAATTA |
| | TATCTTGATGGCAACGCTCGTCAGAACCTAGCCACTTTCTGCCAGACCTG |
| | GGACGACGAAAACGTCCATAAATTGATGGATTTGTCGATCAATAAAAACT |
| | GGATCGACAAAGAAGAATATCCGCAATCCGCAGCCATCGACCTGCGTTGC |
| | GTAAATATGGTTGCCGATCTGTGGCATGCGCCTGCGCCGAAAAATGGTCA |
| | GGCCGTTGGCACCAACACCATTGGTTCTTCCGAGGCCTGTATGCTCGGCG |
| | GGATGGCGATGAAATGGCGTTGGCGCAAGCGTATGGAAGCTGCAGGCAAA |
| | CCAACGGATAAACCAAACCTAGTGTGCGGTCCGGTACAAATCTGCTGGCA |
| | TAAATTCGCCCGCTACTGGGATGTGGAGCTGCGTGAGATCCCTATGCGCC |
| | CCGGTCAGTTGTTTATGGACCCGAAACGCATGATTGAAGCCTGTGACGAA |
| | AACACCATCGGCGTGGTGCCGACTTTCGGCGTGACCTACACCGGTAACTA |
| | TGAGTTCCCACAACCGCTGCACGATGCGCTGGATAAATTCCAGGCCGACA |
| | CCGGTATCGACATCGACATGCACATCGACGCTGCCAGCGGTGGCTTCCTG |
| | GCACCGTTCGTCGCCCCGGATATCGTCTGGGACTTCCGCCTGCCGCGTGT |
| | GAAATCGATCAGTGCTTCAGGCCATAAATTCGGTCTGGCTCCGCTGGGCT |
| | GCGGCTGGGTTATCTGGCGTGACGAAGAAGCGCTGCCGCAGGAACTGGTG |
| | TTCAACGTTGACTACCTGGGTGGTCAAATTGGTACTTTTGCCATCAACTT |
| | CTCCCGCCCGGCGGGTCAGGTAATTGCACAGTACTATGAATTCCTGCGCC |
| | TCGGTCGTGAAGGCTATACCAAAGTACAGAACGCCTCTTACCAGGTTGCC |
| | GCTTATCTGGCGGATGAAATCGCCAAACAGGGGCCGTATGAGTTCATCTG |
| | TACGGGTCGCCCGGACGAAGGCATCCCGGCGGTTTGCTTCAAACTGAAAG |
| | ATGGTGAAGATCCGGGATACACCCTGTACGACCTCTCTGAACGTCTGCGT |
| | CTGCGCGGCTGGCAGGTTCCGGCCTTCACTCTCGGCGGTGAAGCCACCGA |
| | CATCGTGGTGATGCGCATTATGTGTCGTCGCGGCTTCGAAATGGACTTTG |
| | CTGAACTGTTGCTGGAAGACTACAAAGCCTCCCTGAAATATCTCAGCGAT |
| | CACCCGAAACTGCAGGGTATTGCCCAGCAGAACAGCTTTAAACACACCTG |
| | A |

1-59. (canceled)

60. A transgenic Salmonella typhi Ty21a comprising a chromosome with one or more heterologous nucleic acid regions, wherein the one or more heterologous nucleic acid regions encode one or more SARS-CoV-2 viral antigens and are integrated into the Salmonella typhi Ty21a chromosome, and wherein the transgenic Salmonella typhi Ty21a stably expresses the one or more SARS-CoV-2 viral antigens.

61. The transgenic Salmonella typhi Ty21a of claim 60, wherein the one or more heterologous nucleic acid regions comprise a nucleic acid sequence encoding one or more copies of the ectodomain of a SARS-CoV-2 S protein (eS) or one or more antigenic fragments thereof.

62. The transgenic Salmonella typhi Ty21a of claim 61, wherein the one or more heterologous nucleic acid regions comprise a nucleic acid encoding one or more antigens retaining at least 70% amino acid residue homology to a native SARS-CoV-2 eS antigen.

63. The transgenic Salmonella typhi Ty21a of claim 61, wherein the one or more heterologous nucleic acid regions encode a modified SARS-CoV-2 eS.

64. The transgenic Salmonella typhi Ty21a of claim 63, wherein the modified SARS-CoV-2 eS comprises one or more of the following: (i) a D614G substitution, (ii) a GSAS substitution at amino acid residues 986-987, (iii) proline substitutions at amino acid residues 986 and 987, (iv) a C-terminal T4 fibritin trimerization motif, and (v) F817P, A892P, A899P, and A942P substitutions.

65. The transgenic Salmonella typhi Ty21a of claim 61, wherein the heterologous nucleic acid regions comprise a modified SARS-CoV-2 eS encoding the amino acid sequence of SEQ ID NO: 1 or SEQ ID NO: 5.

66. The transgenic Salmonella typhi Ty21a of claim 61, wherein one or more heterologous nucleic acid regions additionally encode one or more SARS-CoV-2 S protein Receptor Binding Domain (RBD).

67. The transgenic Salmonella typhi Ty21a of claim 66, wherein one or more of the encoded SARS-CoV-2 S protein Receptor Binding Domain (RBD) comprises the amino acid sequence of SEQ ID NO:11.

68. The transgenic Salmonella typhi Ty21a of claim 61, wherein the one or more heterologous nucleic acid regions comprise a nucleic acid sequence at least 70%, at least 75%, at least 80%, at least 85%, at least 90% at least 95%, at least 99%, or 100% identical to the nucleic acid sequence of SEQ ID NO: 6.

69. The transgenic Salmonella typhi Ty21a of claim 60, which further comprises a heterologous acid resistance gene cassette integrated into the Salmonella typhi Ty21a chromosome.

70. The transgenic Salmonella typhi Ty21a of claim 61, wherein the one or more heterologous nucleic acid regions comprise a nucleic acid sequence at least 70%, at least 75%, at least 80%, at least 85%, at least 90% at least 95%, at least 99%, or 100% identical to the nucleic acid sequence of SEQ ID NO: 8.

71. The transgenic Salmonella typhi Ty21a of claim 69, wherein the heterologous acid resistance gene cassette comprises a gene selected from the group consisting of: a YbaS gene or a fragment thereof, a GadB gene or fragment thereof, a GadC gene or fragment thereof, a GadA gene or fragment thereof, and any combination thereof.

72. The transgenic Salmonella typhi Ty21a of claim 69, which is more acid stable at pH 2.5 than Salmonella typhi Ty21a without the integrated acid resistance gene.

73. A composition comprising the transgenic Salmonella typhi Ty21a of claim 69 in combination with a carrier suitable for pharmaceutical use.

74. A vaccine comprising the composition of claim 73, which is suitable for oral or inhaled administration.

75. The vaccine of claim 74, which has been foam dried.

76. The vaccine of claim 74, wherein the vaccine is protective against COVID-19.

77. The vaccine of claim 75, wherein the vaccine is at least 80% stable, at least 85% stable, at least 90% stable, at least 95% stable, at least 99% stable, or 100% stable at room temperature for at least one month.

78. An isolated nucleic acid comprising the nucleic acid sequence of SEQ ID NO: 6 or SEQ ID NO: 8, wherein the isolated nucleic acid is codon optimized for expression in Salmonella typhi Ty21a.

79. A method of eliciting an immune response in a subject against a SARS-CoV-2 viral antigen or a modification thereof, the method comprising administering one or more doses of the composition of claim 60.
